(19) United States
(12) Patent Application Publication
(10) Pub. No.: US 2002/0150594 A1
(43) Pub. Date: Oct. 17, 2002

(54) Methods and compositions for developing spore display systems for medicinal and industrial applications

(75) Inventors: Stanley Goldman, Walnut Creek, CA (US); Stephanie J. Lathrop, Sunnyvale, CA (US); Pascal F. Longchamp, East Palo Alto, CA (US); Robert G. Whalen, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Maxygen, Inc., Redwood City, CA

(21) Appl. No.: 10/028,247

(22) Filed: Dec. 19, 2001

Related U.S. Application Data
(63) Continuation-in-part of application No. 09/892,208, filed on Jun. 26, 2001.
(60) Provisional application No. 60/214,161, filed on Jun. 26, 2000.

(51) Int. Cl. A61K 39/02

(52) U.S. Cl. 424/234.1

(57) ABSTRACT

Compositions and methods for utilizing spore systems for medicinal and industrial protein applications are provided. Compositions comprise spores that produce and/or display carbohydrates, proteins, peptides, and nucleic acids of interest. Such spores are useful as therapeutic or prophylactic agents or vaccines against a broad spectrum of immunogens and bacterial and viral pathogens. Additionally, spore systems are useful in production, packaging, delivery, and presentation of polypeptides and/or nucleic acids for industrial catalysts, medical applications, and diagnostic applications.

### CROSS-REFERENCE TO RELATED APPLICATIONS

This application is a Continuation in Part of U.S. Ser. No. 09/892,208, filed Jun. 26, 2001, which claims priority to and benefit of U.S. Provisional Patent Application Serial No. 60/214,161, filed on Jun. 26, 2000, the disclosure of both are incorporated herein by reference in their entirety for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### STATEMENT REGARDING FEDERALLY SPONSORED RESEARCH AND DEVELOPMENT

This work was supported in part by a grant from the Space and Naval Warfare Systems Command (SPAWAR) (Grant No. N65236-99-C-5834). The Government may have certain rights in this invention.

### FIELD OF THE INVENTION

The present invention relates generally to the use of spores as production, packaging, delivery, and presentation systems for industrial biocatalysts and in medical applications, including immunization and vaccination.

### BACKGROUND OF THE INVENTION

Under conditions of a limitation in the supply of carbon, nitrogen, or phosphorous, certain gram-positive rods (aerobic Bacilli and anaerobic Clostridia) and a few sarcinae and actinomycetes form highly resistant, dehydrated forms called endospores or spores. Many gram-positive bacteria share the ability to form such a distinctive type of dormant cell. Bacterial spores can be readily recognized microscopically by their intracellular site of formation, their extreme refractility, and their resistance to staining by basic aniline dyes that readily stain vegetative cells. Other organisms are also capable of forming spores. For example, yeasts, such as the yeast Saccharomyces cerevisiae, form spores.

Spores are not normally formed during active growth and division; rather, the differentiation of spores begins when a population of vegetative cells passes out of the exponential growth phase as a consequence of nutrient limitation. The ability to produce spores involves a complex process of differentiation that begins as the population passes out of exponential growth and approaches the stationary phase. This process leads to the synthesis within most vegetative cells of a new type of cell quite different from the mother cell in structural detail, chemical composition, and physiological properties. Typically, one spore is formed in each vegetative cell. In some organisms, such as the yeast Saccharomyces cerevisiae, sporulation occurs following meiosis and the resulting genetic products of meiosis are packaged individually, so that each vegetative cell undergoing sporulation produces four spores.

In the process of bacterial sporulation, the spore is produced in the mother cell by a consecutive layering of different materials around the replicated genome. The spore development process is regulated temporally by a number of transcription factors as well as a variety of proteins that have roles in scaffolding and packing to create the final spore. The first layers of the spore are comprised of small, acid-soluble proteins that bind to the newly replicated genomic DNA. Subsequently, a membranous material is layered over the developing spore center, followed by a “cortex” composed of layers of peptidoglycans. Finally, the spore is surrounded by the inner and outer spore coats, which are proteinaceous in nature. The outer spore coat is thought to be composed of small, highly cross-linked, insoluble proteins. The inner and outer spore coats perform a role in sensing germination signals and provide the spore with a high degree of resistance to degradation.

The mature spore is eventually released from the vegetative mother cell in which it has developed. Spores have no detectable metabolism and are highly resistant to destructive environmental forces, including ultraviolet and ionizing radiation, many toxic chemicals, and heat. The heat resistance of spores is frequently exploited in the isolation of spore-forming bacteria. Normally, the spore remains dormant for an extended period of time. If subjected to appropriate stimuli, the spore can germinate and grow into a typical vegetative cell. Ungerminated spores retain the capacity to germinate and develop into vegetative cells for years, or even decades.

### SUMMARY OF THE INVENTION

Compositions and methods for utilizing spore systems for medicinal and industrial protein applications are provided. Compositions comprise spores that produce and/or display proteins, polypeptides, peptides, and nucleic acids of interest. Such spores are useful as therapeutic and/or prophylactic agents and as vaccines against a broad spectrum of immunogens and bacterial, viral, and parasitic pathogens and toxins. Additionally, spore systems are useful in production, packaging, delivery, and presentation of polypeptides, proteins, peptides, and/or nucleic acids for industrial catalysts and in medicine. Such spores are also useful as tools in biotechnology applications, such as capture technology and diagnosis.

The invention provides methods for modulation of an immune response of a subject that comprise contacting the subject with a spore system comprising a modified or recombinant spore having at least one exogenous nucleic acid, peptide, polypeptide, or other molecule of interest that modulates an immune response in the subject, wherein the spore is administered via a delivery system selected from the group consisting of respiratory delivery system, nasal delivery system, parenteral delivery system, and mucosal delivery system. An amount of the spore system or molecule of interest is administered that is effective to modulate an immune response.

In another aspect, the invention provides methods for modulation of an immune response of a subject that comprise contacting the subject with a spore system comprising a non-viable modified or recombinant spore having at least one exogenous nucleic acid, peptide, polypeptide, protein, or other molecule of interest that modulates an immune response in the subject. The amount of the spore system or molecule of interest that is administered that is effective to modulate an immune response.

The invention also includes methods of enhancing an immune response to an immunogenic polypeptide (e.g., an antigen) in a subject that comprise administering to the subject a population of spores and an immunogenic polypeptide, wherein the immune response to the immunogenic polypeptide is enhanced compared to the immune response generated by administration of the immunogenic polypeptide alone to the subject. The amount of the spore system or polypeptide that is administered that is effective to enhance the immune response.

In another aspect, the invention provides methods for enhancing an immune response to an immunogenic polypeptide (e.g., antigen) or an expression vector encoding the immunogenic polypeptide in a subject, the method comprising administering to the subject a population of spores and an expression vector comprising a nucleotide sequence encoding the immunogenic polypeptide, wherein the immune response is enhanced compared to the immune response generated by administration of the expression vector or encoded immunogenic polypeptide alone to the subject. The amount of the spore system or encoded polypeptide that is administered that is effective to enhance the immune response.

In yet another aspect, the invention includes compositions comprising a spore system that comprises a spore and at least one peptide, polypeptide, protein, carbohydrate, or nucleotide sequence having anti-pathogenic activity displayed on, bound to, or contained within the spore.

Also included are compositions comprising a spore system that comprises a non-viable spore and at least one exogenous nucleic acid, protein, peptide, or polypeptide displayed on, bound to, or contained within the spore. In addition, the invention provides compositions comprising a spore system that comprises a spore and at least one exogenous nucleic acid binding particle displayed on or bound to the spore.

### BRIEF DESCRIPTION OF THE DRAWINGS

FIG. 1 is a diagram of steps involved in an exemplary screening and selection of spore systems.

FIG. 2 is a diagram of the steps involved in developing an exemplary recombinant library and screening by an in vitro assay. A library of recombinant nucleotide sequences of interest can be generated by shuffling or other methods of generating diversity known to one of skill in the art and discussed elsewhere herein. The library of recombinant molecules is then transformed into a population of cells capable of sporulation. The transformed cells are induced to sporulate, generating a population of spores displaying the peptides, polypeptides, or proteins encoded by the recombinant library. In one embodiment the spores display multiple copies of the same polypeptide. In another embodiment, the spores contain more than one recombinant plasmid each encoding a different polypeptide. In such an embodiment the spores display multiple different peptides, polypeptides, or proteins.

FIG. 3 is a diagram of the steps involved in an exemplary multistep selection process leading to an in vitro test followed by in vivo pathogen challenge to test efficacy of a vaccine produced using a spore system of the present invention.

FIG. 4 depicts flow cytometry analysis of spore surface display of the Yersinia pestis V antigen fused to the CotC spore coat protein of B. subtilis and a viral epitope exemplified by HA11. The fusion protein was detected with monoclonal antibodies directed to HA11 (anti HA11) and polyclonal antibodies directed to the Y. pestis V antigen (anti V antigen). Cells carrying the construct encoding the cotC-V antigen fusion protein (pUSH1.C2.V.pro) are compared with cells lacking the construct (untransformed).

FIG. 5 depicts localization of an exogenous peptide to the spore coat of B. subtilis. Coat proteins were extracted from B. subtilis spores containing constructs encoding spore coat-fusion proteins containing exogenous HA11 epitopes. Coat proteins were extracted using dithiothreitol (DTT), sodium dodecyl sulfate (SDS) and heat. The extracts were fractionated on a 4%-12% Novex bis-tris polyacrylamide gel and transferred to a nitrocellulose membrane. The blot was blocked and then probed with monoclonal antibody (mAb) to HA-11. Lane 1 contains coat proteins from spores containing the B. circulans lipase gene fused to the cotC protein and the HA11 epitope. Lane 2 contains coat proteins from spores containing the cotV gene fused to an HA11 epitope under the control of the cotC promoter. Lane 3 contains coat proteins from spores containing the CotB gene fused to an HA11 epitope under the control of the cotC promoter. In lane 4, molecular weight markers are indicated.

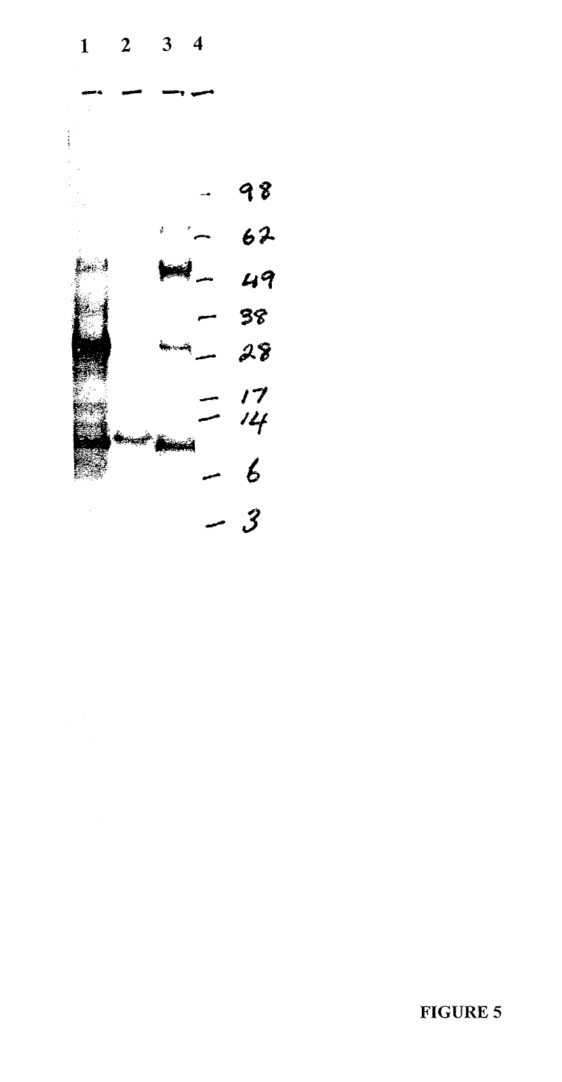

FIG. 6 depicts ELISA analysis of anti-V antigen antibodies in BALB/c mice (n=5 per group) injected with spores displaying recombinant Y. pestis V antigen. Serum was serially diluted in 3-fold dilutions from 1:20 to 1:43740. Closed triangles indicate the mean absorbance at each dilution in response to non-recombinant spores. The absorbance wavelength was 450 nm. Open squares indicate the response to 5×10⁷ spores displaying V antigen. Closed circles represent the response to 5×10⁷ spores displaying V antigen co-administered with adjuvant (monophosphoryl lipid A (MPL) and synthetic trehalose dicorynomycolate in squaline (TDM) (obtained from Sigma)).

FIG. 7 depicts the results of an investigation into the ability of B. subtilis spores to function as an adjuvant. Columns 1 through 3 represent the titer of mouse serum antibodies to three different doses of V-antigen without spores, measured at 3 time points. Columns 4 through 6 represent the titer of mouse serum antibodies to three different doses of V-antigen mixed with 5×10⁸ spores, measured at 3 time points. Each bar represents the geometric mean titer (GMT) for each group of 10 mice, plus/minus the standard deviation.

FIG. 8 depicts the lipase activity of lipase 396 displayed on spores in two conformations. Time points are indicated on the x-axis; hydrolysis product levels are indicated on the y-axis. The solid grey symbols indicate data obtained from clone 16. Clone 16 comprises an insertion of lipase 396 between amino acids 27 and 28 of CotC. The outlined symbols indicate data obtained from clone 19. Clone 19 has a translational terminator after the lipase sequence, terminating translation prior to expression of the remainder of CotC. Results with wild-type spores, serving as the negative control, are also indicated (black symbols). Spore suspensions were incubated with either nerolbutyrate or geranioldeuterobutyrate for 15, 45, 120, or 240 minutes. Reactions were quenched by addition of CHCl₃ prior to gas chromatography/mass spectrometry analysis of the products.

FIG. 9 depicts the titers obtained from oral inoculation of C57BL6 mice. The geometric mean titer (GMT) is displayed on the x-axis.

FIG. 10 depicts the titers obtained from nasal inoculation of C57BL6 mice. The geometric mean titer (GMT) is displayed on the x-axis.

FIG. 11 depicts the results of experiments performed to determine the ability of B. subtilis spores to enhance the immune response to a DNA vaccine. Five mice were injected three times i.m. with the indicated amount of a DNA plasmid encoding the Hepatitis B surface antigen, with and without 5×10⁸ wild-type B. subtilis spores. Sera were collected and assayed for anti-surface antigen immunoglobulin (Ig).

FIGS. 12A-12F depicts flow cytometry analysis of double display of epitopes on the B. subtilis spore surface. The HA11 epitope is fused to cotC and the c-myc epitope is fused to cotV. A and 12B depict spores displaying both HA11 and c-myc. C and 12D depict spores displaying only HA11. E and 12F depict non-recombinant spores. HA11 was detected using fluorescein isothiocyanate (FITC)-labeled antibodies on channel FL1H and c-myc was detected using phycoerythrin (PE)-labeled antibodies on channel FL2H.

FIG. 13 shows analysis of double positive recombinant spores of the invention. Wildtype spores (a and b) and spores displaying both V antigen fused to cotC-HA11 and LT fused to cotV-c-myc (c and d) were stained with anti HA11 (a and c) and anti c-myc (b and d). Wildtype spores were negative, whereas the double display spores demonstrated surface staining of both HA11 and c-myc.

FIG. 14 demonstrates that spores of the invention can be used to induce an immune response. BALB/c mice (n=5) were injected intraperitoneally with spores displaying either V antigen alone (V7) or both V antigen and LT (V7-LT double display). Serum collected from the mice at days 21 and 49 was assayed for anti-V antigen immunoglobulin. The mice injected with V7-LT spores showed approximately 4-fold higher geometric mean titers (GMT) than the mice injected with spores displaying V antigen alone.

FIG. 15 shows that intranasal administration of spores of the invention induces an immune response. 1 of 5 mice had a measurable titer to V antigen following intranasal inoculation with spores displaying V antigen. In contrast, 3 of 5 mice inoculated intranasally with 1×10⁸ LT spores mixed with spores displaying V antigen had measurable titers to V antigen; and 2 of 5 mice inoculated intranasally with 1×10⁷ LT spores mixed with spores displaying V antigen had measurable titers to V antigen.

FIG. 16 shows ELISA analysis of serum from mice inoculated intranasally with spores of the invention. Anti-V antigen ELISA analysis of serum from mice inoculated intranasally with spores displaying V antigen alone (V7), or mixes of spores displaying V antigen and LT.

FIG. 17 shows that spores of the invention can induce a protective immune response. Numbers of mice surviving challenge with Y. pestis from each vaccination group. The group of mice injected with 5×10⁹ recombinant spores (displaying V. antigen) was the only group demonstrating 100% survival. Additionally, the inclusion of wildtype spores with the V antigen protein resulted in increased survival compared to mice receiving protein alone, validating the adjuvant effect discussed in earlier examples.

FIG. 18 shows that spores of the invention administered intranasally can induce a protective immune response. Numbers of mice surviving challenge with Y. pestis following intranasal vaccination with spores displaying V antigen.

FIG. 19 shows FACS analysis of spores of the invention. Wild type spores (A) and spores displaying BT toxin on the surface were stained with a monoclonal antibody specific for HA11. Spores containing the cotC-BT toxin fusion were demonstrated to display the toxin on the surface (B).

FIG. 20 shows the effect of Cry1Ca displayed on the spores of B. subtilis on Spodoptera exigua neonates. The activity of the displayed BT toxin (Cry1Ca) was assayed by mixing a population of recombinant spores displaying either approximately 32 ug BT toxin (“full strength”)or displaying approximately 16 ug BT toxin (“half strength”) with insect larvae (Spodoptera exigua), and applying the mix to leaves and incubating for 96 hours. Insect larvae were also treated with the diluent used as a carrier for the recombinant spores or the insect larvae (“diluent treated” spores). Untreated insect larvae (“untreated”) destroyed the leaves, whereas leaves exposed to the mix of insect larvae and spores were protected, demonstrating that spores displaying BT toxin had anti-insecticidal activity.

FIG. 21 shows FACS analysis of spores of the invention. Untransformed (MW10) Bacillus, MW10 transformed with a plasmid coding for V antigen-cotC fusion (V7), and cw1D-MW10 transformed with the V7 plasmid were stained with a monoclonal antibody to HA11 epitope tag and analyzed by FACS. The peak fluorescence was shifted similarly in the V7 and cw1D-V7 spores, indicating similar amounts of surface display of the recombinant V antigen fused to cotC as fusion protein.

FIG. 22 shows a comparison of the respective responses of different strains of mice to recombinant V antigen from Yersinia pestis fused to cotC coat protein and displayed on B. subtilis spores.

### DETAILED DESCRIPTION OF THE INVENTION

Compositions and methods for utilizing spore systems, spore display systems and spore encapsulate systems for therapeutic, prophylactic, pharmaceutical medicinal, industrial, and other applications are provided. Compositions comprise spores that are modified to display, contain, produce, or express polypeptides, peptides, proteins, carbohydrates, and/or nucleic acids of interest. Such spores are useful in a variety of applications.

As indicated, the spores of the invention are manipulated to display at least one nucleic acid, peptide, polypeptide, protein, bacterium, virus, carbohydrate, or other molecule of interest. The nucleic acid may be DNA, RNA, or derivatives thereof. The peptide, polypeptide, or protein may comprise an antigen, enzyme, immunomodulatory polypeptide, or other protein, polypeptide, or peptide molecule. That is, spores can be used as a delivery platform for the nucleic acids, proteins, peptides, and polypeptides of interest. The spores of the invention are useful in a variety of settings. The uses of the spores will be briefly discussed followed by a general discussion of the spore technology.

General Methods

Therapeutic, Prophylactic, and Medical Applications and Methods

In one embodiment, the modified or recombinant spores are useful as therapeutic and prophylactic agents in therapeutic and prophylactic treatments and as vaccines. For example, protein-, polypeptide-,peptide-, or nucleic acid-displaying spores that produce, stimulate, or invoke an immunomodulatory response(s) in an organism or subject are useful as antigenic agents, therapeutic and prophylactic agents, and vaccines against a broad spectrum of bacterial, viral, and parasitic pathogens and toxins, allergens, cancer-associated antigens and autoantigens. In some such instances, a spore is genetically modified to display or contain at least one nucleic acid molecule, polypeptide, protein, or peptide which produces, invokes, or stimulates an immune response in an organism or subject. Any antigen of interest or antigenic peptide fragment thereof, or multiple antigens or antigenic fragments thereof, can be displayed on the spore to produce an immune response. By “immune response” is intended an alteration of an organism's or subject's immune system in response to an immunomodulatory agent, immunogen, or antigen that may include, but is not limited to, antibody production, induction of cell-mediated immunity, complement activation, development of immunological tolerance, inhibition of an immune response, or breaking of immunological tolerance. An “immunomodulatory agent” modulates an immune response. An “immunogen” refers generally to a substance capable of provoking or altering an immune response, and includes, but is not limited to, e.g., immunogenic proteins, polypeptides, and peptides; antigens and antigenic peptide fragments thereof; and nucleic acids having immunogenic properties or encoding, e.g., polypeptides having such properties.

An “antigen” refers generally to a substance capable of eliciting the formation of antibodies in a host or generating a specific population of lymphocytes reactive with that substance. Antigens may comprise macromolecules (e.g., polypeptides, proteins, and polysaccharides) that are foreign to the host.

As used herein, an “antibody” refers to a protein comprising one or more polypeptides substantially or partially encoded by immunoglobulin genes or fragments of immunoglobulin genes. The term antibody is used to mean whole antibodies and binding fragments thereof. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 KDa) and one “heavy” chain (about 50-70 KDa). The N-terminus of each chain defines a variable region of about 100 to 110 or more amino acids primarily responsible for antigen recognition. The terms variable light chain (VL) and variable heavy chain (VH) refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region. The Fc portion of the antibody molecule corresponds largely to the constant region of the immunoglobulin heavy chain, and is responsible for the antibody's effector function (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies also include single-armed composite monoclonal antibodies, single chain antibodies, including single chain Fv (sFv) antibodies in which a variable heavy and a variable light chain are joined together (directly or through a peptide linker) to form a continuous polypeptide, as well as diabodies, tribodies, and tetrabodies (Pack et al. (1995) J Mol Biol 246:28; Biotechnol 11:1271; and Biochemistry 31:1579). The antibodies are, e.g., polyclonal, monoclonal, chimeric, humanized, single chain, Fab fragments, fragments produced by an Fab expression library, or the like.

“Subject” as used herein includes, but is not limited to, an organism; a mammal, including, e.g., a human, non-human primate (e.g., baboon, orangutan, monkey), mouse, pig, cow, goat, cat, rabbit, rat, guinea pig, hamster, horse, monkey, sheep, or other non-human mammal; and a non-mammal, including, e.g., a non-mammalian vertebrate, such as a bird (e.g., a chicken or duck) or a fish, and a non-mammalian invertebrate.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as, e.g., a nucleic acid, vector, expression cassette, polypeptide, peptide, protein, antigen, substance, spore, spore system, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically agent” or “prophylactically useful” agent or compound refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, expression cassette, polypeptide, protein, peptide, antigen, substance, spore, spore system, or composition thereof, that eliminates or diminishes signs or symptoms of pathology, disease or disorder, when administered to a subject suffering from such signs or symptoms. A “therapeutic agent” or “therapeutically useful” agent or compound indicates that an agent or compound is useful in diminishing, treating, or eliminating such signs or symptoms of a pathology, disease or disorder.

Generally, since the genes involved in spore structure and assembly have been cloned and promoter sequences identified, the appropriate promoter and gene fusion can be selected to control the position, amount, and hence the availability of enzymatic activity or immunomodulatory or antigenic presentation on the spore.

Genetic vaccine and protein-based vaccine and immunomodulatory formulations are also envisioned. Spores that express positive charges on their surface are able to bind nucleic acids, such as double or single-stranded linear DNA, covalently closed plasmid DNA, RNA, or oligonucleotides. Thus in one embodiment of the invention, DNA or plasmid DNA suitable for DNA-based immunization is bound to positively charged spores to create a DNA vaccine formulation or composition. In one aspect, for example, positively charged amino acids are expressed as fusion proteins with spore coat genes, such as e.g., those from the B. subtilis cot gene family. The diameter of spores (ca. 1 micron) readily allows them to be taken up by cells such as macrophages and dendritic cells, carrying the bound nucleic acid into the cells for expression of encoded genes. Such in situ expression initiates an immune response, e.g., to the one or more polypeptides or proteins encoded by the delivered nucleic acid. Envisioned also is the display of general and specific nucleic acid binding particles that allow delivery or capture of nucleic acid molecules in a general or sequence specific manner.

In another embodiment, discussed in greater detail below, a spore is engineered to express a binding molecule, such as avidin or streptavidin, on its surface. With such spores, a wide variety of biotinylated molecules, including, e.g., polypeptides, proteins, peptides, nucleic acids, polysaccharides, bacteria, viruses, small chemical or biological molecules, and other molecules as described herein, can be bound. In such formats, the spore serves as a carrier or delivery device. Thus, in one aspect, the invention provides protein-based vaccine and immunomodulatory compositions comprising spores and spore systems expressing such binding molecules with immunomodulatory molecules or protein-based vaccines bound thereto for use in therapeutic or prophylactic applications.

The spores themselves can be used as an adjuvant for immunomodulatory molecules or vaccines (e.g., genetic vaccines, DNA vaccines, protein vaccines, attenuated or killed viral vaccines). For use as adjuvants, the spores can be modified or recombinant spores, non-modified or non-recombinant spores. Furthermore, for use as adjuvants, any such spores can be viable or non-viable. As used herein, an “adjuvant” is a compound that acts in a non-specific manner to augment specific immunity (e.g., an immune response) to an immunomodulatory molecule, such as, e.g., an immunogenic polypeptide or peptide or antigen, by stimulating an earlier, stronger or more prolonged response to an immunomodulatory molecule. By “adjuvant effect” is intended an augmentation or increase in immunity to an immunomodulatory molecule (e.g., an antigen). See Warren (1992) Roitt et al. eds. Encyclopedia of Immunology 1:28-30.

In one aspect, the invention provides methods of modulating an immune response to an immunomodulatory molecule or vaccine in a subject, the method comprising administering to the subject a population of spores and an immunomodulatory molecule or vaccine, wherein the immune response to the immunomodulatory molecule or vaccine is modulated to a greater or lesser degree as compared to the immune response generated by administration of the immunomodulatory molecule or vaccine alone to the subject.

In some such methods, the spore serves as an adjuvant, acting in a non-specific manner to enhance specific immunity to the immunomodulatory molecule or vaccine by stimulating an earlier, stronger or more prolonged response to the immunomodulatory molecule or vaccine. The spores may comprise viable spores or non-viable or non-germinating spores. The immunomodulatory molecule may comprise, e.g., an immunogenic protein, polypeptide, or peptide; or antigen or fragment thereof, a nucleic acid having immunomodulatory properties; or a nucleotide sequence encoding an immunomodulatory molecule; or the like. The vaccine may comprise, e.g., a genetic vaccine, DNA vaccine, protein-vaccine, or attenuated or killed viral vaccine.

In one aspect, the invention provides methods of enhancing an immune response to an immunomodulatory molecule or vaccine in a subject. Some such methods comprise administering to the subject a population of spores and the immunomodulatory molecule or vaccine, wherein the immune response to the immunomodulatory molecule or vaccine is enhanced compared to the immune response generated by administration of the immunomodulatory molecule alone to the subject. In some such methods, the immunomodulatory molecule or vaccine is a protein, polypeptide, or peptide. In some such methods, the immunomodulatory molecule or vaccine comprises an expression vector comprising a nucleotide sequence encoding an immunomodulatory protein, polypeptide, or peptide, wherein the immune response to such encoded protein, polypeptide, or peptide is enhanced compared to the immune response generated by administration of the expression vector or the encoded protein, polypeptide, or peptide alone to the subject.

In some such methods of the invention, the enhanced immune response comprises an increased production of antibodies specific to the immunomodulatory protein, polypeptide, peptide or antigen that is readily measured by known assays, including those described herein (e.g., ELISA, etc.). Additionally, spores can be prepared that express other immunostimulatory molecules or other molecules involved in determining vaccine effectiveness, such as, e.g., cytokines (e.g., interleukins (IL), interferons (IFN), chemokines, hematopoietic growth factors, tumor necrosis factors and transforming growth factors), which are small molecular weight proteins that regulate maturation, activation, proliferation and differentiation of the cells of the immune system. Such molecules serve as additional immunostimulators for the administered immunomodulatory molecule, protein-based vaccine, DNA vaccine, or viral vaccine.

Exemplary cytokines include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, GM-CSF, G-CSF, TNF-α, IFN-α, IFN-γ, and IL-20 (MDA-7). Antagonists of such cytokines can also be expressed on spores for use as therapeutic and/or prophylactic agents in immunomodulatory methods described herein.

Furthermore, spores can be prepared that express co-stimulatory molecules that play a fundamental role in the regulation of immune responses. Generally speaking, a “co-stimulatory molecule” refers to a molecule that acts in association or conjunction with, or is involved with, a second molecule or with respect to an immune response in a co-stimulatory pathway. In one aspect, a co-stimulatory molecule may be an immunomodulatory molecule that acts in association or conjunction with, or is involved with, another molecule to stimulate or enhance an immune response. In another aspect, a co-stimulatory molecule is immunomodulatory molecule that acts in association or conjunction with, or is involved with, another molecule to inhibit or suppress an immune response. A co-stimulatory molecule need not act simultaneously with or by the same mechanism as the second molecule. Some such co-stimulatory molecules comprise co-stimulatory polypeptides that have positive co-stimulatory properties, such as the ability to stimulate or augment T cell activation and/or proliferation. Membrane-bound co-stimulatory molecules include CD1, CD40, CD154 (ligand for CD40), CD40 ligand, CD27, CD80 (B7-1), CD86 (B7-2) and CD150 (SLAM), and variants or mutants thereof. May such co-stimulatory molecules are typically expressed on lymphoid cells after activation via antigen recognition or through cell-cell interactions.

Alternatively, the immunomodulatory molecules or antigens expressed by spores can be adjuvanted by the further binding of nucleic acid (e.g., plasmid DNA) to positively charged spores. Plasmid DNA has an adjuvant effect either for itself as a DNA vaccine or for proteins with which it is mixed. Plasmid DNAs can be optimized by diversity generation methods (e.g., DNA shuffling) before binding to spores to increase their immunostimulatory potential. Likewise, immunostimulatory oligodeoxynucleotides (ODN) can be bound to positively charged spores that also express immunomodulatory molecules or antigens. These ODNs provide an adjuvant effect when suitable sequences are used. ODNs with a phosphorothioate backbone may be used to increase the in vivo stability of the ODN.

It is also recognized that immunosuppressive ODNs sequences can be used to down-regulate an immune response, for example in the treatment of autoimmune disorders, including allergies, asthma, and the like.

Spores and plasmid DNA are stable to temperature, pH, drying conditions, etc., and thus a positively charged spore-DNA plasmid complex is readily stored. Likewise, positively charged spore-DNA plasmid complexes can be readily formulated for administration to a subject by a variety of means, as described in detail below, including, e.g., as a composition for parenteral, nasal, respiratory, mucosal, vaginal, rectal, or oral administration, including, e.g., as a pill for oral delivery of DNA for mucosal immunization.

As indicated above, heterologous antigens, polypeptides, proteins, and peptides can be attached to the spore outer-coat by creating genetic fusions between outer-coat proteins and target antigens, polypeptides, proteins, or peptides. With the various different coat proteins to attach and display proteins, polypeptides, or peptides, it is recognized that such proteins, polypeptides, or peptides may be displayed in a manner to stretch or torque such sequences, e.g., to expose internal domain surfaces or to change enzyme or antigenic activities. The protein, polypeptide, or peptide of interest can be fused to one coat protein at the amino terminal, may be fused to a coat protein at the carboxyl terminal, may be fused to one coat protein at the amino terminal and a second coat protein at the carboxyl terminal, or may be internally fused to a coat protein. When attached at both ends, as the two coat proteins are assembled into the spore coat, the central protein, polypeptide, or peptide of interest will be stretched.

While any polypeptide, peptide, or protein may be displayed in this manner, one exemplary application is the display of gp120-gp41 fusion from the HIV virus envelope protein so as to effectively display the internal domain of the protein for recognition by immunological systems (e.g., human immunological systems) to produce neutralizing antibodies to the HIV envelope protein. The benefit of this approach is that the domains of the envelope protein are left in a relatively native conformation with the anchors acting to make a previously buried or hidden domain available for the immunogenic machinery of the human immunological system.

For use in industrial settings, as discussed below, the anchoring or stretching of enzymes may change their catalytic properties, including substrate binding specificity and functionality of the catalytic site. The change brought about by the anchoring and steric torquing may change one or both of these characteristics as well as stabilize the protein and its activities for use in biocatalytic processes.

Industrial Applications

The spores are also useful for the production and immobilization of enzymes or proteins for industrial use. That is, the spores find use as an industrial delivery platform for enzymes, binding and capture molecules, and detector reagents. In industrial biocatalysis, the spore may be decorated with a required enzymatic activity. In some instances, production synthesis can be performed that may be otherwise impossible in single organism fermentation runs. Enzymes of industrial relevance may be assembled into the spore outer and inner coat layers as fusion proteins. The modified or recombinant spores can be assayed for expression, stability, and activity. Immobilization of the spore can be accomplished by attachment of modified or recombinant spores to any type of solid support. Appropriate solid supports include, but are not limited to, beads, glass beads, metal beads, membranes, gels, microtiter plates, vessels, containers, pellets, and polymers. Immobilization of the spore system allows repeated uses of the immobilized spore system, although mobile spores may also be reused.

The transformation of a substrate to a desired product in biocatalytic pathway is often a multi-step process requiring multiple enzymes. One of the limiting factors in this kind of enzymatic transformation is the substrate concentration for the intermediate steps. Individual intermediate substrates for transformation into the product each represent a potential limiting component of the entire chemical transformation. The recombinant spores can be used to locally increase the substrate concentrations and thereby greatly increase the reaction rates of each of the intermediate steps increasing yields. In this manner, the different enzymes needed for a particular biocatalytic transformation can all be displayed on a single spore. As discussed in more detail below, there are numerous sites on spores where enzymes can be displayed and many enzymes can be positioned on the same spore. The proximity of catalytic centers acts to increase substrate concentration and enhance the completion rate of multi-step enzymatic transformations.

The topology of the spore surface is highly structured and provides a highly ordered three-dimensional lattice structure. That is, the different coat proteins occupy a specific predetermined and assembled location with respect to each other. This lattice structure defines a certain degree of proximity or distance from coat protein to coat protein. Thus, by cloning the enzymes of a biocatalytic reaction of interest in different locations on the spore coat, the optimal degree of topological proximity for each enzyme leading to the most advantageous production level can be assessed. In this manner, spores can be assembled to maximize biocatalytic reactions.

It is recognized that many modifications of the industrial spore may be made. For example, the enzyme as well as variants of the enzyme may be expressed in multiple locations on the spores. Variants include natural variants from different bacterial species. Alternatively, shuffling of the enzymes may be used to increase the performance of the enzyme constructs. The shuffled constructs can be analyzed by expression and high throughput activity assays. In this manner, platform technology may be developed to improve high throughput analysis of shuffled libraries by using the spore as a display module.

Spores are very small; for example, spores can have a diameter as small as one micrometer. Thus, improved methods of chromatography are provided. For example, a spore system comprising a spore and a polypeptide, protein, peptide, or nucleic acid molecule may be useful in chromatographic applications. In such an application, for example, the spore system serves as a chromatographic matrix with the polypeptide, protein, peptide, or nucleic acid molecule acting as the chemical agent in the separation process.

The recombinant spores if desired can be attached to insoluble support structures in bioreactors. Surface modified spores may also be spread by spraying equipment or from the air. This approach would effectively saturate an environment with degradative or modifying enzymes displayed on the spore surface. Such approaches may be useful in situations such as oil spill cleanups. Additionally, spores are insoluble support structures in their own right. The spores have sufficient density so that they do not float in resting or still solutions. Their density allows the collection of the spore component in a fermentor by allowing the spores to sink to the bottom at the end of a synthesis thus creating a situation where centrifugation is not required to remove the spores from the reactor.

Generally, the recombinant spores of the invention can be used in many industrial settings including, industrial fermentation reactions, industrial column reactors, cleanups, bioremediation of organic solvents and heavy metals, as delivery systems in agricultural applications, and the like. Thus, one of skill in the art recognizes that the enzyme will vary depending upon the application.

Spore Systems and Spore Display Systems as Diagnostic Tools

Spore systems are also useful as sensors and detectors. For example, such systems may comprise a spore having two different components. In one embodiment, one such component captures a detectable compound, while the other component is a moiety that provides a detectable signal indicating that interaction between the first component and the target molecule has occurred (such as, for example, fluorescence, phosphorescence, color change, or other indication). By “captures” a detectable compound is intended a selective interaction including, but not limited to, binding, linking or contacting the detectable compound. Such a target molecule is a detectable compound selected from the group comprised of, but not limited to, proteins, polypeptides, peptides, nucleic acid, nucleotides, nucleosides, amino acids, phospholipids, cations, anions, enzymatic substrates, antibodies, antigenic agents, ligands and antagonists. Spore systems, spore display systems, and spore encapsulate systems of the present invention may utilize either non-viable or viable spores.

Examples of moieties that provide a detectable signal include, but are not limited to, various enzymes, prosthetic groups, fluorescent materials, luminescent materials, bioluminescent materials, radioactive materials, chromophores, and fluorophores. Suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; examples of suitable radioactive material include ¹²⁵I, ¹³¹I, ³⁵S, or ³H; examples of chromophores include green fluorescent protein (GFP), blue fluorescent protein, and DsRed; and examples of fluorophores include BoDIPY, fluorescein, Oregon green, rhodamine, and Texas Red. See, for example, The Handbook of Fluorescent Probes and Research Products, 8^{th} Ed. Molecular Probes, Inc.

Other Applications

The modified or recombinant spores of the invention find use in a variety of biotechnology settings. In one embodiment, the spores are useful as carriers for nucleic acids and biotin-linked ligands. One such method involves the expression of positively charged amino acids as fusions with proteins found on the exterior of spores, chosen, for example, from those proteins encoded by the cot gene family. Spores that express positive charges on their surface bind nucleic acids, including single- or double-stranded linear DNA, covalently closed plasmid DNA, RNA and oligonucleotides. A number of the Cot proteins have a high percentage of positively charged amino acids, therefore unmodified spores may be sufficiently positively charged to bind DNA. Expression of additional positively charged amino acid sequences, such as poly-lysine, would increase the capacity of the spores for binding nucleic acids. An alternative approach, for example, comprises making genetic fusions between the genes encoding small acid soluble proteins (SASPs), HU histone like protein from thermophilic bacteria (Esser et al., J Mol Biol (19991)291:1135-1146), and a cot gene. Surface display of the SASPs, whose function is to bind DNA, would make a highly effective nucleic acid binding particle.

Specific and generic binding proteins can also be used to enhance the intended activities of spores. The use of proteins such as single-stranded binding protein (SSBP) from E. coli and the Bacillus SASPs can give spores the capacity to bind DNA in ways that are not sequence specific. SSBP expressed on the surface of a spore would preferentially bind single-stranded DNA molecules while SASP would preferentially bind double-stranded DNA.

In the same manner, proteins with very specific DNA or RNA sequence-binding affinity may be used to attach only specific nucleic acid types to the spore surface alone or in combinations with other components as described above. Additionally, proteins that specifically bind RNA can be used.

The display of proteins with sequence-specific DNA-binding activities can be used in a variety of settings including binding plasmids at specific sequences so as to ensure that only the specified DNA is bound to the spore giving greater control and purity of delivered DNA. In addition to specificity the spore displaying sequence specific DNA binding proteins can be used to capture the DNA thereby acting as a receptor component of a detector.

In another embodiment, avidin (e.g., chicken egg-white avidin) or streptavidin (e.g., from Streptomyces) can be expressed on the spore surface. The high affinity of biotin for avidin or streptavidin has made such pair of molecules very useful for numerous applications, including in vivo, ex vivo, or in vitro uses. The creation of such “avidin spores” (or “streptavidin spores”) provides a generic reagent to which a wide variety of biotinylated molecules can be bound. For example, chicken avidin is rich in basic residues and its isoelectric point is pH 10. It is therefore positively charged at neutral pH. Expression and display of avidin on spores results in spores that can bind biotin and are also positively charged. Since a family of avidin-related genes exists in the chicken genome, recombination (such as, e.g., by DNA shuffling) of such genes can be carried out to obtain a chimeric avidin molecule that demonstrates high and specific binding of biotin once expressed on the surface of the spore. A number of examples of the use of the avidin spores (or streptavidin spores) can be envisioned including: binding biotinylated positively charged small molecules, for example chloroquine, to avidin decorated spores that bind or display another molecule (Upon uptake of the complex into an acidic cellular vesicle compartment, the vesicular pH will be increased and entry to the cytoplasm will be facilitated.); spores decorated with biotinylated enzymes to be used in chemical reactors (Settling, centrifugation, or filtration will remove the spore-enzyme complex after completion of the reaction. The tight binding of the enzyme to the spore through the high affinity avidin-biotin link prevents any of the enzymes from remaining in the reaction mixture.); coupling biotin to a polypeptide linker containing a proteolytic site that separates the avidin decorated spores from the molecule of interest (Upon uptake into cells, the linker is cleaved releasing the molecule of interest into the cell, free of the spore components.); delivery of biotinylated peptide nucleic acids (PNA) which bind to DNA and RNA with high specificity to cells using avidin decorated spores (A cleavable peptide linker can be used to allow the PNA to be released from the spore component.); biotinylated peptides, polypeptides, or proteins can be complexed with avidin decorated spores for vaccine delivery (Since the spores are taken up by antigen-presenting cells (APCs), this represents a method to cause peptides, polypeptides, or proteins to enter the class I and II processing pathways for the induction of helper T cells and cytotoxic T lymphocytes.); complex antigens that are poorly immunogenic, e.g., an inactivated virus or bacterial preparation, can be biotinylated and complexed to avidin decorated spores (The ability of spores to be taken up by APCs enhances the immunogenicity of the antigen preparation. The avidin decorated spores can also bind, express, or display other molecules that will further enhance the immunogenicity of the antigen complex.); polysaccharides can be coupled to biotin and then complexed with avidin decorated spores to enhance their immunogenicity (The avidin decorated spores can also express or display other molecules that further enhance the immunogenicity of the polysaccharide complex.); biotin can be coupled to a positively charged molecule such as poly-lysine and bound to avidin decorated spores (Nucleic acids can then be bound to the positively charged avidin decorated spore-biotin-poly-lysine complex.).

In another application, linear double-stranded nucleic acid (e.g., DNA) with biotin bound to a 5′ or 3′ end can be coupled directly to avidin-spores for delivery to cells. One aspect of this approach involves the use of linear expression elements (LEE). (See, Sykes et al. (1999) Nature Biotechnology 17:355-359) Using this method, any open-reading frame (ORF) can be amplified by polymerase chain reaction (PCR) and noncovalently linked to a eukaryotic promoter and terminator. The resulting linear expression element (LEE) can be directly injected into animals to produce local gene expression, which results in an immune response to the encoded and expressed protein. By adding biotin (e.g., using biotinylated PCR primers) to the promoter, terminator or ORF, the LEEs can be bound to spores and delivered as a complex, thus enhancing the efficiency of delivery relative to pure linear double-stranded DNA. An extension of this approach involves preparing a reassembled complex of the avidin-decorated spores and the biotinylated promoter element of the LEE. After PCR amplification, the ORF would then be added to this preformed complex.).

In yet another application, immunostimulatory oligodeoxynucleotides (ODN) labeled with biotin can be bound to avidin decorated spores that also express antigens (The ODNs provide an adjuvant effect when suitable sequences are used. Alternatively, immunosuppressive ODN sequences can be used to down-regulate an immune response, for example in the treatment of autoimmunity of allergy); etc.

It is recognized that free avidin remaining on spores can be saturated with biotin before administration as a medicament in order to avoid any possibility of biotin depletion due to the avidin decorated spores.

Because spores are particularly adapted for prolonged survival under adverse conditions, spore-based methods can fill needs that remain unmet by other expression systems. In industrial enzyme applications, for example, spores are relatively insensitive to damage from shearing forces or chemical degradation. Thus, while vegetative cells in bioreactors can rapidly die and degrade, causing expensive shutdown, cleanup, and restarting of a bioreactor process, spore systems can be used with or without attachment to insoluble or solid support structures in bioreactors. In this way, the spore structure can be used essentially as an immobilization substrate that can be adapted to many different matrices (see, for example, U.S. Pat. No. 5,766,914 and U.S. Pat. No. 5,348,867). Thus, bioreactors utilizing spore systems can last longer and provide more reliable sources of enzyme.

The invention also includes a spore system comprising having two or more or a library of molecules of interest displayed or expressed on the spore surface. (A library typically comprises at least two or more members. A library of nucleic acids comprises at least 2, 5, 10, 20 or 50 or more nucleic acids.) For example, a library of recombinant vectors, each comprising, e.g., a gene encoding a coat protein and at least one nucleotide sequence encoding at least one molecule of interest (e.g., antigen) is used to transform a population of spores, such that a library of different molecules of interest (e.g., different antigens) are expressed or displayed on the surface of the spores. In one aspect, individual spores of the population of spores each express or display different molecules (e.g., different antigens or antigen variants). In another aspect, one or more spores of the population of spores each express or display two or more different molecules of interest; in this format, at least two different antigens or antigen variants are displayed on the surface of a spore. Thus, the population of spores may express a diverse library of recombinant antigen variants.

A library of recombinant expressed vectors (e.g., plasmids) may be constructed using a library of recombinant nucleotide sequences encoding variants of a polypeptide of interest. Such variants can be generated by any of a variety of means, including, e.g., by using one of the diversity generation procedures described herein, such as shuffling. If desired, the recombinant nucleotide sequences are cloned into expression vectors comprising at least one coat protein gene sequence (e.g., CotC). Alternatively, such recombinant nucleotide sequences are cloned into expression vectors comprising different coat protein gene sequences (e.g., CotC, CotG, CotD, etc., gene sequences).

A spore system displaying two or more different polypeptides of interest, or a combination of different spore systems—e.g., a combination of two or more spore systems, each displaying one or more different polypeptides of interest—is useful in a variety of therapeutic and prophylactic therapies. For example, there are four major serotypes of Dengue virus, and a tetravalent vaccine or immunomodulatory composition that induces neutralizing Abs against all four serotypes is necessary. Moreover, non-neutralizing antibodies induced by infection or vaccination by one Dengue virus may cause enhancement of the disease during a subsequent infection by another serotype. Thus, a cross-protective, broad-spectrum vaccine for the four Dengue serotypes would provide a significant improvement over existing vaccines. A spore system displaying antigens to the four Dengue serotypes, or a combination of different spore systems—each spore system displaying an antigen to one of the four Dengue serotypes—would be useful as a vaccine or immunomodulatory agent and in methods to induce a cross-protective immune response against the four Dengue serotypes.

Such spore systems and combinations thereof comprising various combinations of antigenic variants would also be useful as genetic or protein vaccines effective in inducing immune responses against a group of antigenically related viruses, such as flaviviruses, which consist of the following three clusters of: Dengue 1-4 (62-77% identity), Japanese, St. Louis and Murray Valley encephalitis viruses (75-82% identity), and the tick-borne encephalitis viruses (77-96% identity).

In another format, one or more expression vectors are prepared in which each vector comprises at least two coat genes and an insertion of at least one nucleic acid sequence into each, each of which encodes a different polypeptide variant. In such format, each such vector co-expresses each such at least two polypeptide variants. When a population of spores is transformed with such an expression vector, the at least two polypeptide variants are co-expressed and displayed on the spore surfaces. Alternatively, each expression vector is constructed to include a coat gene and one nucleic acid sequence encoding only one polypeptide variant.

Spore systems that display an adjuvant and polypeptide of interest (e.g., antigen) can also be constructed by transforming a spore with a plasmid prepared by inserting nucleic sequences encoding such molecules, e.g., into a coat protein gene or at the N or C-terminus of a coat protein gene.

Having generally discussed the uses of the modified and recombinant spores of the invention, more details on their construction and use are provided below.

Recombinant and Modified Spores and Spore Systems

As used herein, “spore system” refers collectively to recombinant spores, modified spores, spore display systems, and spore encapsulate systems of the present invention as described herein. By “spore display system” is intended to include a spore system of the present invention wherein the bacterial or fungal spore comprises or has at least one nucleic acid or vector (e.g., expression vector or expression cassette) encoding one or more polypeptides, proteins, or peptides of interest; a spore system comprising or having at least one nucleic acid of interest that is bound to, associated with, expressed on, displayed on, or provided on the surface of the spore; a spore system comprising at least one polypeptide, protein, or peptide of interest bound to, associated with, expressed on, presented on, displayed on, or provided on the surface of the spore; a spore system comprising at least one nucleic acid, polypeptide, protein, peptide, virus, bacterium, carbohydrate, or other molecule of interest, including those described herein, that has been chemically coupled to the surface of the spore; and combinations of one or more of these and compositions comprising any of these. Display on the surface of the spore allows one or more polypeptides, proteins, peptides, nucleic acids, viruses, bacteria, polysaccharides, small chemical or biological molecules, and other molecules of interest to interact with the environment or with target molecules in the environment. Such spore display systems also allow for the administration of such molecules of interest and local or site-specific delivery of such molecules to subjects in a variety of applications, including vaccine applications and therapeutic and prophylactic treatment protocols. By “spore encapsulate system” is intended that polypeptides, proteins, peptides, polynucleotides, nucleic acids, and other molecules of interest, including those described herein, are encapsulated within the spore (e.g., within the outer coat, inner coat, and/or cortex and/or in the core) by virtue of association with one or more of the native polypeptides located within the outer coat, inner coat, cortex, and/or core of the spore. This association can be either covalent, e.g., as a fusion protein, or non-covalent, e.g., by electrostatic or other secondary interaction. By “having” is intended throughout to mean comprising, displaying, encapsulating, containing, carrying, holding, associated with, is attached to, binding or bound to, or is coated with.

The term “nucleic acid” or “nucleic acid molecule” refers generally to deoxyribonucleotides or ribonucleotides and polymers thereof in either single- or double-stranded form, including molecules of DNA, RNA, a synthetic analog, or a combination thereof. The term is used interchangeably with gene, CDNA, and mRNA encoded by a gene. Unless specifically limited, the term encompasses nucleic acids containing known analogues of natural nucleotides that have similar properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Cassol et al. (1992); Rossolini et al. (1994) Mol Cell Probes 8:91-98).

A vector is a component or composition for facilitating cell transduction or transfection by a selected nucleic acid, or expression of the nucleic acid in the cell. Vectors include, e.g., plasmids, cosmids, viruses, YACs, bacteria, poly-lysine, etc. An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter. The nucleic acid to be transcribed is typically under the direction or control of the promoter.

An “expression cassette” or “recombinant expression cassette” is a nucleic acid construct, generated recombinantly or synthetically, with nucleic acid elements that are capable of effecting expression of a structural gene in hosts compatible with such sequences. An expression cassette includes at least a promoter(s) and optionally, transcription termination signal. Typically, the expression cassette includes a nucleic acid to be transcribed (e.g., a nucleic acid encoding a desired polypeptide, protein, or peptide), and a promoter. Additional factors necessary or helpful in effecting expression may also be used as described herein. For example, an expression cassette can also include a nucleotide sequence that encodes a signal sequence that directs secretion of an expressed protein from the host cell. Transcription termination signals, enhancers, and other nucleic acid sequences that influence gene expression, can also be included in an expression cassette. pore systems, spore display systems, and spore encapsulate systems can be used as spore delivery systems, optionally together with the related mother cell, for the controlled release of proteins, polypeptides, peptides, nucleic acids, polysaccharides, small chemical molecules, biological molecules, including vitamins, and other molecules of interest at a site of interest. For example, controlled delivery of polypeptides, peptides, proteins, nucleic acids and other molecules of interest can be achieved by the controlled lysing of a vegetative mother cell containing the spore systems of the present invention. Alternatively, controlled delivery from spore encapsulate systems can be accomplished by allowing spores of a spore system to germinate and produce the molecule(s) of interest (e.g., polypeptide, protein, or peptide) of interest during the germination process, give rise to vegetative cells that produce the molecule(s) of interest, or release such molecules from the core of the spore. In these ways, the spore systems of the present invention provide a means for controlling the delivery of nucleic acids, polypeptides and other molecules of interest to a target site. This control of delivery encompasses the timing and the location of delivery of polypeptides, polynucleotides, nucleic acids, and other molecules of interest. This controlled delivery of such molecules may be useful in many situations and processes where controlled delivery of such molecules is advantageous (e.g., controlled delivery of an immunomodulatory agent, vaccine composition component, or molecule (e.g., protein) used to a “boost” a vaccine's effectiveness. For example, it may be useful to deliver molecules having biocatalytic activities to a biochemical synthesis or degradation reaction in a controlled manner, e.g., in a bioreactor, at a bioremediation site, in a cleaning formulation, etc.

Spore systems in which polypeptides, proteins, peptides, polynucleotides, and/or nucleic acid molecules of interest are displayed on, stored within, or expressed by the mother cell, the spore, or cells arising from the spore after germination are provided by the present invention. Genes involved in spore synthesis and structure have been identified and cloned, and promoter sequences from such genes have been isolated and characterized. One of skill in the art will appreciate that by selecting among these promoters and regulatory sequences, it is possible to govern the physical location of expression of the polypeptide of interest in the spore or vegetative cell as well as the timing of expression in the life cycle of the spore and/or vegetative cell. See, for example, Hill et al., Soc. Appl. Bacteriol. Symp. Ser. 23: 129S-134S (1998), demonstrating that model proteins such as luciferase and beta-galactosidase can be directed to the endospore during the sporulation process by operably linking the nucleic acid sequences encoding these proteins to sporulation-specific promoters. Hill et al. further demonstrates that these model enzymes are stored effectively and retain their activities.

Display of at least one polypeptide, protein, peptide, or nucleic acid of interest on the surface of the spore or vegetative cells arising therefrom is provided by the present invention. The present invention provides expression cassettes or constructs comprising a promoter operably linked to or fused in frame with a nucleic acid molecule encoding a polypeptide, protein, or peptide of interest, which may further be operably linked with a nucleic acid molecule encoding a part or all of a spore coat gene. An expression cassette or construct may also comprise a promoter operably linked to a multiple cloning site that may further be operably linked with nucleic acid molecule encoding a part or all of a spore coat gene. By “multiple cloning site” is intended a nucleic acid sequence comprising one or more restriction enzyme sites. Spore coat genes may be selected from the group consisting of, but not limited to, cotA, cotB, cotC, cotD, cotE, cotF, cotG, cotN, cotS, cotT, cotV, cotW, cotX, cotY, and cotZ. See, for example, Donovan, et al. (1987) J. Mol. Biol. 196:1-10; Zheng, et al. (1988) Genes and Develop. 2:1047-1054; Cutting et al. (1991) J. Bacteriol 1 73:2915-2919; Arnson et al. (1989) Mol. Microbiol. 3:437-444; Zhang, et al. (1993) J. Bacterial 1 75:3757-3766; Kunst et al. (1997) Nature 390 (6657): 249-56. In the current best mode of practice, the sporulation-preferred promoter region from cotC regulates transcription of the nucleic acid molecule of interest.

By “exogenous” is intended that the polypeptide, peptide, protein, or nucleic acid molecule(s) displayed on, stored within, or expressed by the spore system is not normally found in the spore but rather has been added by some means. That is, by “exogenous” is intended that the expressed polypeptide, peptide, protein, or nucleic acid molecule has been introduced into the spore system by genetic manipulation of the spore system. For example, the polypeptide, peptide, protein, or nucleic acid molecule may be added into the spore system by genetic transformation, as part of an expression vector. In another embodiment, the polypeptide, peptide, protein, or nucleic acid molecule is introduced by a transduction process from another organism, such as bacteria, that has previously been genetically transformed.

The expression cassette is then introduced into the bacteria and the resulting strain induced to sporulate; these fusion proteins are efficiently directed to the spore coat. Similarly, vegetative cells arising from spores may express polypeptides, proteins, or peptides of interest within their cytoplasm and may secrete such molecules. For example, fusion proteins with vegetative surface proteins may direct expression to the vegetative cell surface; these proteins may then be used for their intended purpose while still attached to the vegetative surface. Alternatively, these proteins may be released from the vegetative surface to perform an application.

The spore systems of the present invention may be used with any organism that is capable of forming spores (e.g., bacteria or fungus such as, for example, yeast and the like). Thus, any of a variety of spore-forming organisms may be useful in practicing the methods and compositions of the present invention. For example, spore-forming bacteria which may be useful in practicing the present invention include, but are not limited to, Clostridium botulinum, Clostridium lentoputrescens, Clostridium perfringens, Clostridium sporogenes, Clostridium tetuni, and Bacillus species, for example Bacillus anthracis, Bacillus coagulans, Bacillus globigii, Bacillus stearothermophilus, Bacillus thuringiensis, and Bacillus subtilis.

By “bacterial strains” as used herein is intended any bacterial species capable of spore formation as well as any bacteria derived therefrom which contain mutations in one or more genes. Thus, bacterial strains useful for spore systems of the invention include strains that contain mutations in genes involved in normal development of the spore inner and/or outer coat. For example, a Bacillus strain utilized in the spore display system of the current invention may contain a mutation in the CotE or the GerE gene, or both, thereby producing a spore with additional exposed surfaces relative to the wildtype Bacillus strain. Strains containing cotE or gerE mutations, for example, may have enhanced utility in spore display or delivery systems of the present invention because changes to the outer and/or inner spore coat may result in slightly different conformation and activity or antigenicity of an expressed polypeptide. Similar mutations identified in other bacterial species or strains may be useful in the practice of the present invention. Further, useful mutations may be mutations that alter the process of sporulation of the bacteria. For example, useful mutations may affect formation of the spore coat. In one embodiment, delivery formulations may be better suited for particular applications when spores with altered properties are used to create the spore display or spore delivery system of the present invention. For example, a spore with a mutant, fragile spore coat may lyse or burst open with relative ease compared to wildtype spores and therefore be a better means of encapsulation and delivery of an enzyme produced in the spore interior or of antigenic delivery. Further, bacterial strains containing mutations having other phenotypes may be useful in embodiments of the present invention. For example, a bacterial strain containing thermosensitive mutations which modulate metabolic requirements or synthetic capacities can be used to alter the timing of delivery of polypeptides of interest.

The coding sequences of the invention are typically provided in vectors, such as, e.g., expression vectors or expression cassettes, for expression in the organism or host cell of interest. The vector or cassette typically includes 5′ and 3′ regulatory sequences operably linked to a nucleotide sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The expression vector or cassette can be incorporated into a plasmid, chromosome, mitochondrial DNA, plastid DNA, virus, transposon, or nucleic acid fragment. The expression vector or cassette may additionally contain at least one additional gene to be cotransformed into the organism. Alternatively, the additional gene(s) can be provided on multiple expression vectors or cassettes.

Such an expression vector or cassette is provided with a plurality of restriction sites for insertion of the coding sequence to be under the transcriptional regulation of the regulatory regions. The expression vector or cassette may additionally contain selectable marker genes.

The expression vector or cassette usually includes in the 5′-3′ direction of transcription one or more of the following: a transcriptional and translational initiation region, a nucleotide sequence of the invention, and a transcriptional and translational termination region functional in plants or in the desired organism. The transcriptional initiation region(s) and the promoter(s) may be native or analogous or foreign to the plant host or other type of host. Additionally, the promoter(s) may be the natural sequence or alternatively a synthetic sequence. By “foreign” is intended that the transcriptional initiation region(s) is not found in the native plant or host organism into which the transcriptional initiation region is introduced. As used herein, a chimeric gene comprises at least one coding sequence operably linked to at least one transcription initiation region that is heterologous to the at least one coding sequence.

While it may be preferable to express the sequences using heterologous promoters, the promoter sequences used to regulate expression of the nucleotide sequences of the invention may be used.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed organism. That is, the nucleotide sequences can be synthesized using codons that allow preferred expression in the organism of interest and thereby allow improved expression. See, for example, Campbell and Gowri (1990) Plant Physiol. 92:1-11 for a discussion of host-preferred codon usage. Methods are available in the art for synthesizing bacteria-preferred genes. See, for example, U.S. Pat. Nos. 5,380,831, and 5,436,391, and Murray et al. (1989) Nucleic Acids Res. 17:477-498, herein incorporated by reference in its entirety for all purposes.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression vectors or cassettes may additionally contain 5′ leader sequences in the expression vector or cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression vector or cassette, the various DNA component sequences may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression vector or cassette comprises a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), kanamycin resistant (Kana^{r}), as well as genes conferring resistance to herbicidal compounds, such as glufosinate ammonium, bromoxynil, imidazolinones, and 2,4 dichlorophenoxyacetate (2,4-D). See generally, Yarranton (1992) Curr. Opin. Biotech. 3:506-511; Christopherson et al. (1 992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. In addition, a DNA construct may contain selectable marker genes appropriate for non-plant host cells, for example, E. coli cells. Such disclosures are herein incorporated by reference in their entirety for all purposes. The above list of selectable marker genes is not meant to be limiting. Any selectable marker gene can be used in the present invention.

As used herein, “promoter” includes reference to a region of DNA upstream from the start of transcription and involved in recognition and binding of RNA polymerase and other proteins to initiate transcription. A “sporulation preferred promoter” is a promoter capable of initiating transcription upon or during sporulation. Exemplary sporulation preferred promoters include, but are not limited to, those that are obtained from the regulatory region of nucleotide sequences encoding spore coat proteins. A “constitutive” promoter is a promoter that is active under most environmental conditions.

Expression of Polypeptides, Proteins, Peptides, and Nucleic Acids Using Spore Systems

It is recognized that four different functional regions of the spore can be used in the practice of the invention: the outer coat, the inner coat, the cortex and the core of the spore. Polypeptides, peptides, proteins, or nucleic acids of interest can be displayed on and/or associated with one or more of these four functional regions. For example, a polypeptide of interest can be displayed on, bound to, or associated with the outer surface of the spore. By “spore surface” is intended the exterior layer of the spore that is exposed to the surrounding environment. The spore surface typically comprises an outer coat protein layer. However, spore structure can be genetically altered so as to exclude the outer coat during formation; in this instance, the inner coat protein layer remains and constitutes the spore surface protein layer. A spore structure can be genetically altered to exclude the inner coat and outer coat; in this instance, the cortex proteins constitute the “surface” proteins.

A polypeptide, protein, or peptide of interest is typically displayed on, bound to, or associated with the spore surface by chemical or physical interaction with a spore component (e.g., as fusion protein by covalent attachment of the polypeptide, protein, or peptide of interest to an outer coat protein, inner coat protein, cortex protein, or core protein). Such fusion proteins are typically produced in the cytoplasm of the mother cell and incorporated into or assembled with other spore components when the spore is formed in the mother cell. Additionally, a polypeptide, protein, or peptide of interest can be displayed on, bound to, or associated with the surface of the spore by physical attraction or non-covalent attachment to a component to a spore component (e.g., by electrostatic attraction, Van der Waals interactions, protein-protein interactions, cross-linking, or other secondary interactions).

A nucleic acid of interest is typically displayed on, associated with, and/or bound to the surface of the spore by chemical or physical interactions with a spore component (e.g., by electrostatic attraction, Van der Waals interactions, protein-nucleic acid binding, cross-linking, transesterification, or other secondary interactions). In another embodiment, a nucleic acid binding particle such as a peptide, polypeptide, or protein of interest displayed on the spore surface possesses nucleic acid binding characteristics. The spore system is combined with a nucleotide sequence of interest and the nucleotide binding domain binds the nucleotide sequence of interest, such as an expression vector, an antisense molecule, or DNA antigen. The DNA binding domain of a peptide, polypeptide, or protein displayed on the spore surface may bind a DNA molecule of interest. (For a description of DNA binding domains, see Leon et al. (2000) Biol Res 33:21-30; Robinson et al. (2000) Nucleic Acid Research 28:1499-1505; Segal et al. (2000) Curr. Opin. Chem. Biol. 4:34-39; Suck (1997) Biopolymers 44:405-421; and Creighton, (1993) 2^{nd} Ed. Proteins: Structures & Molecular Properties.) Additionally, the displayed peptide, polypeptide, or protein may comprise an RNA binding domain that binds RNA molecules of interest. (For a description of RNA binding domains, see Blencowe et al. (1999) Biochem Cell Biol 77:277-291; Kim et al. (1998) Amino Acids 15:291-306; Varani et al. (1998) Annu Rev Biophys Biomol Struct 27:407-445; and Creighton, (1993) 2^{nd} Ed. Proteins: Structures & Molecular Properties.) The nucleic acid binding domain may bind nucleic acid molecules in a sequence specific or sequence independent manner. Such nucleic acid molecules of interest may include, but are not limited to, expression vectors or cassettes, antisense molecules, or DNA based antigens. Said expression vector or expression cassette is typically not expressed until the spore system comprising the associated nucleic acid is delivered to a target or target site of interest (e.g., cell or organism).

Carbohydrates can be linked to spores via chemical coupling. For example, this can be accomplished using ABH (available from Pierce, cat. #21510). The carbohydrate is oxidized to form an aldehyde, reacted with the hydrazide group on ABH, and then an arylazide group on the other end of the linker reacts nonspecifically with spore proteins upon UV photolysis. Alternatively, the linkage can be accomplished using MPBH (available from Pierce, cat. #22305). This crosslinker consists of a hydrazide on one end, similar to ABH, and a maleimide on the other end that is a sulfhydryl-reactive group to yield thioether linkages with proteins, e.g. proteins of the spore coat. See O'Shannessy, et al. (1985) J. Appl. Biochem. 7:347-355 and Chamow, et al. (1992) J. Biol. Chem. 267:15916-15922.

A polypeptide, protein, or peptide of interest can also be included within, associated with, and/or bound to the outer coat, inner coat, cortex or core region by chemical (e.g., covalent) or physical interaction (e.g., non-covalent) with an outer coat, inner coat, cortex, or core protein (e.g., by forming a fusion protein with an outer coat, inner coat, cortex, or core protein or by electrostatic interaction with an outer coat, inner coat, cortex or core protein). A nucleic acid of interest can similarly be included within, associated with, and/or bound to an outer coat, inner coat, cortex or core region by physical interaction (e.g., electrostatic attraction, Van der Waals interactions, or other secondary interaction) with an outer coat, inner coat, cortex, or core protein. Polypeptides, carbohydrates, proteins, peptides, or nucleic acids of interest can also be included or stored inside the core of the spore to control delivery to a target site and/or the timing of release at a target site.

During formation of the spore, a polypeptide, protein, or peptide of interest expressed within the cytoplasm of the mother cell is packaged with or assembled into the appropriate spore layer (e.g., outer coat, inner coat, cortex) by, e.g., covalent or non-covalent association with one or more native proteins in a spore layer. Alternatively, a polypeptide, protein, or peptide of interest is packaged within the core of the spore. A polypeptide, protein, or peptide of interest can be expressed either individually or as a fusion protein, preferably as a fusion protein with a particular spore coat protein. By “spore coat protein” is intended any full-length protein that localizes to the spore coat, and any fragment or variant thereof that retains the ability to localize to the spore coat.

In one embodiment, a spore system comprises bacterial spores displaying a protein, polypeptide, peptide, or nucleic acid of interest on the surface of the spore and is used to stimulate an immune response in vivo, ex vivo, or in vitro. In another embodiment, a spore system is used to provide an enzyme to an industrial process (e.g., a biosynthesis process, a bioremediation process, etc.) or product (e.g., a detergent formulation, a reagent, a kit, etc.). The spore system can optionally be used as a delivery vehicle for a protein, peptide, polypeptide, antigen, or nucleic acid of interest. The enzyme may be displayed on the spore surface or may be encapsulated inside the spore; alternatively, the spore may germinate and give rise to vegetative cells that produce the desired enzyme. A vegetative cell can replicate by division and a new spore may be formed inside each new vegetative cell.

The spore coat consists of multiple spore coat proteins that can be used to generate multiple fusion proteins expressing more than one nucleotide sequence of interest. The spore coat polypeptide targets the fusion protein to the spore coat thus exposing the fusion protein on the exterior of the spore. Additionally, there are multiple sites within each spore coat sequence that allow insertion of a nucleotide sequence of interest. The expression vectors or expression cassettes of the invention comprise insertion loci. By “insertion loci”, “display loci”, or “display positions” is intended any sequence of a nucleic acid molecule into which a heterologous nucleotide sequence of interest may be cloned that, when expressed, causes the heterologous peptide, polypeptide, or protein to be targeted to the spore surface. The insertion loci allow expression of a fusion protein between a nucleotide sequence of interest and a spore coat polypeptide. The current invention provides multiple insertion loci. A sporulating organism may be transformed with more than one expression vectors or cassette into which a nucleotide sequence of interest may be cloned. Multiple expression vectors or cassettes may be provided on contiguous or discontiguous nucleic acid molecules (e.g. on the same plasmid or on separate plasmids). Thus, a spore system of the invention may comprise one or more spore expressing one or more exogenous peptide sequences encoded by one or more exogenous nucleotide sequence. A spore system of the invention may comprise one or more spores displaying 1, 2, 3, 4, 5, 6, 7, 8, 9, 10, 11, 12, 13, 14, 15, 16, 17, 18, 19, 20, or more exogenous peptides, polypeptides, proteins, carbohydrates, or nucleotide sequences of interest. A spore system of the invention may comprise a mixture of spores displaying different peptides, polypeptides, proteins, carbohydrates, or nucleotide sequences of interest.

Polynucleotides of Interest

By “polynucleotides” is intended that a nucleotide molecule of the invention may correspond to a full-length, native gene, or it may be a fragment thereof. “Polynucleotides” includes variants of native or naturally occurring nucleotide molecules and can be fragments or analogues of such molecules. Thus, polynucleotide sequences may be entirely synthetic in nature. Polynucleotides and nucleic acid molecules need not encode a polypeptide. Polynucleotides may encode polypeptides having biological activity; however, polynucleotides may not comprise an open reading frame or encode a polypeptide having biological activity. Polynucleotides and nucleic acid molecules may themselves have biological activity. For example, a polynucleotide may have the biological activity of stimulating a response in a cell contacted by the polynucleotide. By “nucleic acid sequence,” “nucleic acid molecule,” or “nucleic acids” is intended that a sequence or molecule may comprise DNA or RNA or synthetic analogues thereof or any of these in combination. Thus, polynucleotides as used herein fall within the definition of nucleic acid molecule. “Nucleic acid molecule” or “nucleic acids” includes variants, fragments, and analogues of naturally occurring nucleic acid molecules.

When used with regard to a polynucleotide or nucleic acid sequence, by “fragment” is intended that a sequence comprises a part of a larger sequence such as, for example, a full-length, native nucleic acid sequence, gene sequence, or polynucleotide sequence. A fragment can correspond to a C-terminal deletion or N-terminal deletion of the full-length native sequence, or it can correspond to both a C-terminal and an N-terminal deletion of the native sequence. A nucleic acid or polynucleotide fragment may be of any length. Thus, a nucleic acid or polynucleotide fragment may be 2 or 5 or 7 residues in length, or 10, 15, 20, or 25 residues in length, or 50, 100, 150, 200, 250, or 300 residues in length, or 400, 500, 600, 700, or more residues in length, or 800, 900, or 1000 residues in length, or 2000, 3000, 4000, 5000, or 6000 residues in length, or it may have a greater length. Thus, the term “polynucleotides” includes oligonucleotides.

By “analogue” is intended an analogue of the native nucleic acid or polynucleotide sequence or molecule, where the analogue comprises a native sequence and structure having one or more substitutions, insertions, or deletions. By “variants” is intended substantially similar sequences. For nucleotide sequences, conservative variants of a native nucleotide sequence include those sequences that, because of the degeneracy of the genetic code, encode the same amino acid sequence as the native nucleotide sequence. Variants such as these can be identified with the use of well-known molecular biology techniques. Variant nucleotide sequences also include synthetically derived nucleotide sequences, such as those generated, for example, by using site-directed mutagenesis. Generally, variants of a particular nucleotide sequence of the invention have at least about 40%, 50%, 60%, 65%, 70%, generally at least about 75%, 80%, 85%, preferably at least about 90%, 91%, 92%, 93%, 94%, 95%, 96%, 97%, 98%, 99% or more sequence identity to that particular nucleotide sequence.

Methods for making polynucleotides and nucleic acid molecules, including polynucleotide and nucleic acid fragments, variants, and analogues, are generally available in the art. For example, variants of the nucleic acid molecule can be prepared by generating mutations in the sequence of the cloned nucleic acid molecule by mutagenesis. Methods for mutagenesis and nucleic acid sequence alterations are well known in the art. See, for example, Walker and Gaastra, eds. (1983), Techniques in Molecular Biology (MacMillan Publishing Company, New York); Sambrook et al. (1989) Molecular Cloning: A Laboratory Manual (2^{nd} edition) (Cold Spring Harbor, New York).

“Nucleic acid molecules” or “nucleic acids” also includes randomized assemblies of nucleic acid residues, whether synthesized de novo or produced by a recombination or mutagenesis process of nucleic acid molecules followed by expression in vitro, ex vivo, or in vivo. Such a recombination or mutagenesis process may include “shuffling” of polynucleotide-encoding nucleic acid molecules. Polynucleotides of interest may be identified by screening libraries of candidate nucleic acid molecules by transformation and expression in bacterial strains. Polynucleotides of interest include antigens; bacterial strains displaying antigens that are recognized by particular antibodies may be created and identified with the methods and compositions of the present invention. Thus, the creation and identification of polynucleotides of interest may be accomplished by mutagenesis of individual sequences or libraries in combination with selection or screening for polynucleotides of interest by functional assay. Mutagenesis may be accomplished by completely synthetic means, and synthetically created sequences may be used in the practice of the current invention.

Suitable polynucleotides and nucleic acids or nucleic acid molecules employed in the practice of the present invention include those identified from natural diversity or prepared and/or identified via diversity-generating procedures, such as those described herein. Methods of identifying polynucleotides of interest are also provided. Polynucleotides of interest may be identified by screening a library of candidates for a desired polynucleotide function, as described herein. A spore system strain used in such an embodiment may produce non-viable spores; alternatively, the spores of such a strain may be viable.

Polypeptides of Interest

“Polypeptides of interest” or “polypeptides” as used herein refers to full-length native proteins, partial proteins or protein fragments, or peptides or polypeptides or polypeptide fragments. “Proteins” and “polypeptides” include suitable biologically active variants of native or naturally occurring proteins and can be fragments, analogues, and derivatives of such proteins. Such biological activity may be any biological activity. For example, such biological activity may be an enzymatic activity or it may be the ability to alter or modulate an immune response in a subject. Thus, a polypeptide, protein, or peptide of the present invention may be an enzyme, such as, for example, laccase. In another embodiment, a polypeptide, protein, or peptide of the present invention is molecule capable of augmenting an immune response, such as, e.g., an antigen or an adjuvant.

Polypeptides, proteins, and peptides of interest include, but are not limited to, cytokines, antigens, antibodies, binding receptors, defensive agents, anti-microbial agents, immunomodulatory molecules, co-stimulatory molecules, enzymes, and epitopes. “Epitope” typically refers to a amino acid (e.g., protein) determinant capable of specific binding to an antibody. Epitopes usually consist of chemically active surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents.

A “subsequence” or “fragment” is any portion of an entire sequence, up to and including the complete sequence. For example, when used with regard to a polypeptide, by “fragment” or “subsequence” is intended that a polypeptide sequence comprises a part of a larger polypeptide sequence such as, for example, a full-length, native protein or polypeptide. A polypeptide or protein fragment can be a C-terminal deletion or N-terminal deletion of the native polypeptide, or which can be both a C-terminal and an N-terminal deletion of the native polypeptide. A protein or polypeptide fragment may be of any length. Thus, a protein or polypeptide fragment may be 2 or 5 or 7 amino acids in length, or 10, 15, 20, or 25 amino acids in length, or 50, 100, 150, 200, 250, or 300 amino acids in length, or 400, 500, 600, 700, or more amino acids in length, or 800, 900, or 1000 amino acids in length, or may have a greater length. By “analogue” is intended an analogue of either the native polypeptide or of a fragment of the native polypeptide, where the analogue comprises a native polypeptide sequence and structure having one or more amino acid substitutions, insertions, or deletions. “Muteins,” such as those described herein, and peptides having one or more peptoids (peptide mimics) are also encompassed by the term analogue (see International Publication No. WO 91/04282). By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. By “variants” is intended substantially similar sequences. By “variant” protein is intended a protein derived from another protein by deletion (so-called “truncation”) or addition of one or more amino acids to the N-terminal and/or C-terminal end of the original protein; deletion or addition of one or more amino acids at one or more sites in the original protein; or substitution of one or more amino acids at one or more sites in the original protein. Variants may result from, for example, genetic polymorphism or from human manipulation. Biologically active variants of a native protein of the invention have at least 40%, 50%, 60%, 70%, generally at least 75%, 80%, 85%, preferably about 90% to 95% or more, and more preferably about 98% or more sequence identity to the amino acid sequence for the native protein as determined by standard sequence alignment programs using default parameters. A biologically active variant of a native protein may differ from that protein by as few as 1-15 amino acid residues, as few as 1-10, such as 6-10, as few as 5, as few as 4, 3, 2, or even 1 amino acid residue. Similarly, a “subsequence” or “fragment” of a nucleic acid sequence is any portion of the entire nucleic acid sequence, up to and including the complete sequence. Methods for making polypeptides, including polypeptide fragments, analogues, and derivatives, are generally available in the art. Thus, a polypeptide of the present invention includes polypeptide fragments such as, for example, epitopes. For example, amino acid sequence variants of the polypeptide can be prepared by generating mutations in the cloned DNA sequence encoding the native polypeptide of interest by mutagenesis. Methods for mutagenesis and nucleic acid sequence alterations are well known in the art. See, for example, Walker and Gaastra, eds. (1983), Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2^{nd} edition) (Cold Spring Harbor, New York); U.S. Pat. No. 4,873,192; and references cited therein. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.). Conservative substitutions, such as exchanging one amino acid with another having similar properties, may be preferred. Examples of conservative substitutions include, but are not limited to, Gly for Ala, Val for Ile, Ile for Leu, Asp for Glu, Lys for Arg, Asn for Gln, Phe for Trp, and Trp for Tyr.

“Polypeptides” also includes randomized assemblies of peptide fragments, whether synthesized de novo or produced by a recombination or mutagenesis process of nucleic acid sequences followed by expression in vitro, ex vivo, or in vivo. Polypeptides of interest may be identified by screening libraries of candidate nucleic acid sequences by transformation and expression in bacterial strains. Polypeptides of interest include antigens and adjuvants. Bacterial strains displaying antigens that are recognized by particular antibodies may be created and identified with the methods and compositions of the present invention. Thus, the creation and identification of polypeptides of interest may be accomplished by mutagenesis of individual sequences or libraries in combination with selection or screening for polypeptides of interest by functional assay. Mutagenesis may be accomplished by completely synthetic means, and synthetically created sequences may be used in the practice of the current invention.

Suitable peptides and polypeptides employed in the practice of the present invention include those identified from natural diversity or prepared and/or identified via diversity-generating procedures, such as those described herein. Methods of identifying polypeptides of interest are also provided. Polypeptides of interest may be identified by screening a library of candidates for a desired polypeptide function, as described herein. In one embodiment of this method, a vaccine antigen is identified by cloning an antigen library into an appropriate spore system strain wherein the antigens are displayed on the spore surface, and cells of the strain are then further screened and tested for antigenic activity. A spore system strain used in such an embodiment may produce non-viable spores; alternatively, the spores of such a strain may be viable.

Diversity-Generating Protocols for Polynucleotides, Nucleic Acids, and Polypeptides, etc.

A variety of diversity-generating protocols, including nucleic acid shuffling protocols, are available and fully described in the art. The following publications describe a variety of recursive recombination procedures and/or methods which can be incorporated into such procedures, as well as other diversity-generating protocols: Stemmer, et al. (1999), “Molecular breeding of viruses for targeting and other clinical properties,” Tumor Targeting 4: 1-4, Ness et al. (1999) “DNA Shuffling of subgenomic sequences of subtilisin,” Nature Biotechnology 17: 893-896; Chang et al. (1999), “Evolution of a cytokine using DNA family shuffling,” Nature Biotechnology 17: 793-797; Minshull and Stemmer (1999), “Protein evolution by molecular breeding,” Current Opinion in Chemical Biology 3: 284-290; Christians et al. (1999), “Directed evolution of thymidine kinase for AZT phosphorylation using DNA family shuffling,” Nature Biotechnology 17: 259-264; Crameri et al. (1998), “DNA shuffling of a family of genes from diverse species accelerates directed evolution,” Nature 391: 288-291; Crameri et al. (1997), “Molecular evolution of an arsenate detoxification pathway by DNA shuffling,” Nature Biotechnology 15: 436-438; Zhang et al. (1997), “Directed evolution of an effective fucosidase from a galactosidase by DNA shuffling and screening,” Proceedings of the National Academy of Sciences, U.S.A. 94: 4504-4509; Patten et al. (1997), “Applications of DNA Shuffling to Pharmaceuticals and Vaccines,” Current Opinion in Biotechnology 8: 724-733; Crameri et al. (1996), “Construction and evolution of antibody-phage libraries by DNA shuffling,” Nature Medicine 2: 100-103; Crameri et al. (1996), “Improved green fluorescent protein by molecular evolution using DNA shuffling,” Nature Biotechnology 14: 315-319; Gates et al. (1996), “Affinity selective isolation of ligands from peptide libraries through display on a lac repressor ‘headpiece dimer,’” Journal of Molecular Biology 255: 373-386; Stemmer (1996), “Sexual PCR and Assembly PCR,” pp. 447-57 in The Encyclopedia of Molecular Biology (VCH Publishers, New York); Crameri and Stemmer (1995), “Combinatorial multiple cassette mutagenesis creates all the permutations of mutant and wildtype cassettes,” BioTechniques 18: 194-195; Stemmer et al., (1995) “Single-step assembly of a gene and entire plasmid from large numbers of oligodeoxyribonucleotides,” Gene 164: 49-53; Stemmer (1995), “The Evolution of Molecular Computation,” Science 270: 1510; Stemmer (1995), “Searching Sequence Space,” Bio/Technology 13: 549-553; Stemmer (1994), “Rapid evolution of a protein in vitro by DNA shuffling,” Nature 370: 389-391; and Stemmer (1994), “DNA shuffling by random fragmentation and reassembly: In vitro recombination for molecular evolution,” Proceedings of the National Academy of Sciences, U.S.A. 91: 10747-10751.

Additional details regarding DNA shuffling and other diversity generating methods are found in U.S. patents by the inventors and their co-workers, including: U.S. Pat. No. 5,605,793 to Stemmer (Feb. 25, 1997), “Methods for in Vitro Recombination;” U.S. Pat. No. 5,811,238 to Stemmer et al. (Sep. 22, 1998) “Methods for Generating Polynucleotides Having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction,” and U.S. Pat. No. 5,837,458 to Minshull, et al. (Nov. 17, 1998), “Methods and Compositions for Cellular and Metabolic Engineering.”

In addition, details and formats for DNA shuffling and other diversity generating protocols are found in a variety of PCT and foreign patent application publications, including: Stemmer and Crameri, “DNA Mutagenesis by Random Fragmentation and Reassembly,” WO 95/22625; Stemmer and Lipschutz “End Complementary Polymerase Chain Reaction,” WO 96/33207; Stemmer and Crameri, “Methods for Generating Polynucleotides Having Desired Characteristics by Iterative Selection and Recombination,” WO 97/0078; Minshull and Stemmer, “Methods and Compositions for Cellular and Metabolic Engineering,” WO 97/35966; Punnonen et al. “Targeting of Genetic Vaccine Vectors,” WO 99/41402; Punnonen et al. “Antigen Library Immunization,” WO 99/41383; Punnonen et al. “Genetic Vaccine Vector Engineering,” WO 99/41369; Punnonen et al. “Optimization of Immunomodulatory Properties of Genetic Vaccines,” WO 9941368; Stemmer and Crameri, “DNA Mutagenesis by Random Fragmentation and Reassembly,” EP 0934999; Stemmer, “Evolving Cellular DNA Uptake by Recursive Sequence Recombination,” EP 0932670; Stemmer et al., “Modification of Virus Tropism and Host Range by Viral Genome Shuffling,” WO 99/23107; Apt et al., “Human Papillomavirus Vectors,” WO 9921979; Del Cardayre et al. “Evolution of Whole Cells and Organisms by Recursive Sequence Recombination,” WO 98/31837; Patten and Stemmer, “Methods and Compositions for Polypeptide Engineering” WO 98/27230; and Stemmer et al., “Methods for Optimization of Gene Therapy by Recursive Sequence Shuffling and Selection,” WO98/13487.

Certain U.S. patent applications provide additional details regarding DNA shuffling and related techniques, as well as other diversity generating methods, including “Shuffling of Codon Altered Genes” by Patten et al. filed Sep. 29, 1998, (U.S. Ser. No. 60/102,362), Jan. 29, 1999 (U.S. Ser. No. 60/117,729), and Sep. 28, 1999, (U.S. Ser. No. 09/407,800) (Attorney Docket Number 20-28520US/PCT); “Evolution of Whole Cells and Organisms by Recursive Sequence Recombination”, by del Cardyre et al. filed Jul. 15, 1998 (U.S. Ser. No. 09/166,188) and Jul. 15, 1999 (U.S. Ser. No. 09/354,922); “Oligonucleotide Mediated Nucleic Acid Recombination” by Crameri et al., filed Feb. 5, 1999 (U.S. Ser. No. 60/118,813), filed Jun. 24, 1999 (U.S. Ser. No. 60/141,049), and filed Sep. 28, 1999 (U.S. Ser. No. 09/408,392) (Attorney Docket Number 02-29620US); and “Use of Codon-Based Oligonucleotide Synthesis for Synthetic Shuffling” by Welch et al., filed Sep. 28, 1999 (U.S. Ser. No. 09/408,393, Attorney Docket Number 02-010070US); “Methods for Making Character Strings, Polynucleotides & Polypeptides Having Desired Characteristics” by Selifonov and Stemmer, filed Feb. 5, 1999 (U.S. Ser. No. 60/118,854) and U.S. Ser. No. 09/416,375 filed Oct. 12, 1999; and “Single-Stranded Nucleic Acid Template-Mediated Recombination and Nucleic Acid Fragment Isolation” by Affholter (U.S. Ser. No. 60/186,482), filed Mar. 2, 2000.

As review of the foregoing publications, patents, published applications and U.S. patent applications reveals, recursive recombination of nucleic acids to provide new nucleic acids with desired properties can be carried out by a number of established methods and these procedures can be combined with any of a variety of other diversity generating methods.

In brief, several different general classes of recombination methods are applicable to the present invention and set forth in the references above. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined ex vivo or in vivo, e.g., by allowing recombination to occur between nucleic acids in cells. Third, whole genome recombination methods can be used in which whole genomes of cells or other organisms are recombined, optionally including spiking of the genomic recombination mixtures with desired library components. Fourth, synthetic recombination methods can be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made by tri-nucleotide synthetic approaches. Fifth, in silico methods of recombination can be effected in which genetic algorithms are used in a computer to recombine sequence strings which correspond to nucleic acid homologues (or even non-homologous sequences). The resulting recombined sequence strings are optionally converted into nucleic acids by synthesis of nucleic acids that correspond to the recombined sequences, e.g., in concert with oligonucleotide synthesis/gene reassembly techniques. Any of the preceding general recombination formats can be practiced in a reiterative fashion to generate a more diverse set of recombinant nucleic acids. Sixth, methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be used.

The above references provide these and other basic recombination formats as well as many modifications of these formats. Regardless of the format which is used, the nucleic acids of the invention can be recombined (with each other or with related (or even unrelated) to produce a diverse set of recombinant nucleic acids, including, e.g., sets of homologous nucleic acids.

Following recombination, any nucleic acids which are produced can be selected for a desired activity. In the context of the present invention, this can include testing for and identifying any activity that can be detected in an automatable format, by any of the assays in the art. A variety of related (or even unrelated) properties can be assayed for, using any available assay. These methods are automated according to the present invention as described herein.

Generation of Diverse Polynucleotides of Interest

DNA mutagenesis and shuffling provide a robust, widely applicable, means of generating diversity useful for the engineering of proteins, pathways, cells and organisms with improved characteristics. In addition to the basic formats described above, it is sometimes desirable to combine shuffling methodologies with other techniques for generating diversity. In conjunction with (or separately from) shuffling methods, a variety of diversity generation methods can be practiced and the results (i.e., diverse populations of nucleic acids) screened for in the systems of the invention. Additional diversity can be introduced by methods that result in the alteration of individual nucleotides or groups of contiguous or non-contiguous nucleotides, i.e., mutagenesis methods.

Mutagenesis methods include, for example: recombination (PCT/US98/05223; Publ. No. WO98/42727); site-directed mutagenesis (Ling et al. (1997), “Approaches to DNA mutagenesis: an overview,” Anal Biochem. 254(2): 157-78; Dale et al. (1996), “Oligonucleotide-directed random mutagenesis using the phosphorothioate method,” Methods Mol Biol. 57:369-74; Smith (1985), “In vitro mutagenesis,” Ann. Rev. Genet. 19: 423-462; Botstein and Shortle (1985), “Strategies and applications of in vitro mutagenesis,” Science 229: 1193-1201; Carter (1986), “Site-directed mutagenesis,” Biochem J. 237: 1-7; Kunkel (1987), “The efficiency of oligonucleotide directed mutagenesis,” in Nucleic Acids & Molecular Biology, Eckstein and Lilley, eds. (Springer Verlag, Berlin)); mutagenesis using uracil-containing templates (Kunkel (1985), “Rapid and efficient site-specific mutagenesis without phenotypic selection,” Proc. Natl. Acad. Sci. USA 82: 488-492; Kunkel et al. (1987), “Rapid and efficient site-specific mutagenesis without phenotypic selection,” Methods in Enzymol. 154: 367-382; Bass et al. (1988), “Mutant Trp repressors with new DNA-binding specificities,” Science 242: 240-245); oligonucleotide-directed mutagenesis (for review, see Smith (1985), Ann. Rev. Genet. 19: 423-462; Botstein and Shortle (1985), Science 229: 1193-1201; Carter (1986), Biochem. J. 237: 1-7; Kunkel (1987), “The efficiency of oligonucleotide directed mutagenesis,” in Nucleic Acids & Molecular Biology, Eckstein and Lilley, eds., (Springer Verlag, Berlin)); Zoller and Smith (1983), “Oligonucleotide-directed mutagenesis of DNA fragments cloned into M13 vectors,” Methods in Enzymol. 100: 468-500; and Zoller and Smith (1987), “Oligonucleotide-directed mutagenesis: a simple method using two oligonucleotide primers and a single-stranded DNA template,” Methods in Enzymol. 154: 329-350; Zoller and Smith (1982), “Oligonucleotide-directed mutagenesis using M13-derived vectors: an efficient and general procedure for the production of point mutations in any DNA fragment,” Nucleic Acids Res. 10: 6487-6500; Zoller and Smith (1983), “Oligonucleotide-directed mutagenesis of DNA fragments cloned into Ml 3 vectors,” Methods in Enzymol. 100: 468-500; Zoller and Smith (1987), “Oligonucleotide-directed mutagenesis: a simple method using two oligonucleotide primers and a single-stranded DNA template,” Methods in Enzymol. 154: 329-350); phosphorothioate-modified DNA mutagenesis (Taylor et al. (1985), “The use of phosphorothioate-modified DNA in restriction enzyme reactions to prepare nicked DNA,” Nucl. Acids Res. 13: 8749-8764; Taylor et al. (1985), “The rapid generation of oligonucleotide-directed mutations at high frequency using phosphorothioate-modified DNA,” Nucl. Acids Res. 13: 8765-8787 (1985); Nakamaye and Eckstein (1986), “Inhibition of restriction endonuclease Nci I cleavage by phosphorothioate groups and its application to oligonucleotide-directed mutagenesis,” Nucl. Acids Res. 14: 9679-9698; Sayers et al. (1988), “Y-T exonucleases in phosphorothioate-based oligonucleotide-directed mutagenesis,” Nucl. Acids Res. 16: 791-802; Sayers et al. (1988), “Strand specific cleavage of phosphorothioate-containing DNA by reaction with restriction endonucleases in the presence of ethidium bromide” Nucl. Acids Res. 16: 803-814); mutagenesis using uracil-containing templates (Kunkel (1985), Proc. Nat'l. Acad. Sci. USA 82: 488-492 and Kunkel et al.(1987), Methods in Enzymol. 154: 367-382)); mutagenesis using gapped duplex DNA (Kramer et al.(1984), “The gapped duplex DNA approach to oligonucleotide-directed mutation construction,” Nucl. Acids Res. 12: 9441-9456; Kramer and Fritz (1987), “Oligonucleotide-directed construction of mutations via gapped duplex DNA,” Methods in Enzymol. 154: 350-367; Kramer et al. (1988), Nucl. Acids Res. 16: 7207); Fritz et al. (1988), “Oligonucleotide-directed construction of mutations: a gapped duplex DNA procedure without enzymatic reactions in vitro,” Nucl. Acids Res. 16: 6987-6999; Isaac and Farah (1988), “Combined PCR/gapped-duplex method for site-directed mutagenesis,” Biotechniques 25(5): 758-60, 762; Kramer et al. (1988), “Improved enzymatic in vitro reactions in the gapped duplex DNA approach to oligonucleotide-directed construction of mutations,” Nucleic Acids Res. 16: 7207; and Bass et al. (1988), “Mutant Trp repressors with new DNA-binding specificities,” Science 242: 240-245).

Additional suitable methods include point mismatch repair (Kramer et al. (1984), “Point mismatch repair,” Cell 38: 879-887), mutagenesis using repair-deficient host strains (Carter et al. (1985), “Improved oligonucleotide site-directed mutagenesis using M13 vectors,” Nucl. Acids Res. 13: 4431-4443; Carter (1987), “Improved oligonucleotide-directed mutagenesis using M13 vectors,” Methods in Enzymol 154: 382-403), deletion mutagenesis (Eghtedarzadeh and Henikoff (1986), “Use of oligonucleotides to generate large deletions,” Nucl. Acids Res. 14: 5115), restriction-selection and restriction-selection and restriction-purification (Wells et al. (1986), “Importance of hydrogen-bond formation in stabilizing the transition state of subtilisin,” Phil. Trans. R. Soc. Lond. A 317: 415-423), mutagenesis by total gene synthesis (Nambiar et al. (1984), “Total synthesis and cloning of a gene coding for the ribonuclease S protein,” Science 223: 1299-1301; Sakamar and Khorana (1988) “Total synthesis and expression of a gene for the a-subunit of bovine rod outer segment guanine nucleotide-binding protein (transducin),” Nucl. Acids Res. 14: 6361-6372; Wells et al. (1985), “Cassette mutagenesis: an efficient method for generation of multiple mutations at defined sites,” Gene 34: 315-323; and Grundström et al. (1985), “Oligonucleotide-directed mutagenesis by microscale ‘shot-gun’ gene synthesis,” Nucl. Acids Res. 13: 3305-3316), double-strand break repair (“Band aid”) (Mandecki (1986), “Oligonucleotide-directed double-strand break repair in plasmids of Escherichia coli: a method for site-specific mutagenesis,” P.N.A.S. 83: 7177-7181). Additional details on many of the above methods can be found in Methods in Enzymology, volume 154, which also describes useful controls for trouble-shooting problems with various mutagenesis methods.

Kits for mutagenesis are commercially available. For example, kits are available from, e.g., Stratagene (e.g., QuickChange site-directed mutagenesis kit; Chameleon double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5′ 3′ prime kit); Genpak Inc, Lemargo Inc, Life Technologies (Gibco BRL), New England Biolabs, Pharmacia Biotech, Promega Corp., Quantum Biotechnologies, Amersham International, plc (e.g., using the Eckstein method above), and Anglian Biotechnology ltd (e.g., using the Carter/Winter method above).

In addition, any of the described shuffling techniques can be used in conjunction with procedures that introduce additional diversity into a genome, e.g. a bacterial genome. For example, techniques have been proposed which produce chimeric nucleic acid multimers suitable for transformation into a variety of species, including E. coli and B. subtilis (see e.g., U.S. Pat. No. 5,756,316 (Schellenberger)). When such chimeric multimers consist of genes that are divergent with respect to one another, (e.g., derived from natural diversity or through application of site directed mutagenesis, error prone PCR, passage through mutagenic bacterial strains, and the like), are transformed into a suitable host, an additional source of nucleic acid diversity for DNA shuffling is introduced. Chimeric multimers transformed into host species are particularly suitable as substrates for in vivo shuffling protocols. Alternatively, a multiplicity of polynucleotides sharing regions of partial sequence similarity can be transformed into a host species and recombined ex vivo or in vivo by the host cell. Subsequent rounds of cell division can be used to generate libraries, members of which, each comprise a single, homogenous population of monomeric or pooled nucleic acid. Alternatively, the monomeric nucleic acid can be recovered by standard techniques and recursively recombined in any of the described shuffling formats.

Chain termination methods of diversity generation have also been proposed (see e.g., U.S. Pat. No. 5,965,408). In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., uv-, gamma-, or X-ray-irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are chimeric with respect to the starting population of DNA molecules. Optionally, the products or partial pools of the products can be amplified at one or more stages in the process. Polynucleotides produced by a chain termination method, such as described above are suitable substrates for DNA shuffling according to any of the described formats.

Diversity can be further increased by using methods which are not homology based with DNA shuffling (which, as set forth in the above publications and applications can be homology or non-homology based, depending on the precise format). For example, incremental truncation for the creation of hybrid enzymes (ITCHY) described in Ostermeier et al. (1999), “A combinatorial approach to hybrid enzymes independent of DNA homology,” Nature Biotech 17:1205, can be used to generate an initial recombinant library which serves as a substrate for one or more rounds of in vitro, ex vivo, or in vivo shuffling methods.

Methods for generating multispecies expression libraries have been described (e.g., U.S. Pat. Nos. 5,783,431 and 5,824,485) and their use to identify protein activities of interest has been proposed (U.S. Pat. No. 5,958,672). Multispecies expression libraries are, in general, libraries comprising cDNA or genomic sequences from a plurality of species or strains, operably linked to appropriate regulatory sequences, in an expression vector or expression cassette. The cDNA and/or genomic sequences are optionally randomly concatenated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the shuffling methods herein described.

In some applications, it is desirable to preselect or prescreen libraries (e.g., an amplified library, a genomic library, a cDNA library, a normalized library, etc.) or other substrate nucleic acids prior to shuffling, or to otherwise bias the substrates towards nucleic acids that encode functional products (shuffling procedures can also, independently have these effects). For example, in the case of antibody engineering, it is possible to bias the shuffling process toward antibodies with functional antigen binding sites by taking advantage of in vivo recombination events prior to DNA shuffling by any described method. For example, recombined CDRs derived from B-cell cDNA libraries can be amplified and assembled into framework regions (e.g., Jirholt et al. (1998), “Exploiting sequence space: shuffling in vivo formed complementarity determining regions into a master framework,” Gene 215: 471) prior to DNA shuffling according to any of the methods described herein.

Libraries can be biased towards nucleic acids that encode polypeptides and proteins with desirable enzyme activities. For example, after identifying a clone from a library which exhibits a specified activity, the clone can be mutagenized using any known method for introducing DNA alterations, including, but not restricted to, DNA shuffling. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations that exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids that have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single-stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom.

Second-strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in a shuffling-based gene reassembly process. In one such method the fragment population derived the genomic library(ies) is annealed with partial, or, often approximately full-length ssDNA or RNA corresponding to the opposite strand. Assembly of complex chimeric genes from this population is mediated by nuclease-base removal of non-hybridizing fragment ends, polymerization to fill gaps between such fragments and subsequent single stranded ligation. The parental strand can be removed by digestion (if RNA or uracil containing), magnetic separation under denaturing conditions (if labeled in a manner conducive to such separation) and other available separation/purification methods. Alternatively, the parental strand is optionally co-purified with the chimeric strands and removed during subsequent screening and processing steps. As set forth in “Single-Stranded Nucleic Acid Template-Mediated Recombination and Nucleic Acid Fragment Isolation” by Affholter (U.S. Ser. No. 60/186,482), filed Mar. 2, 2000, shuffling using single-stranded templates and nucleic acids of interest which bind to a portion of the template can also be performed.

In a conventional approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences that hybridize to the probe. A library produced in this manner provides a desirable substrate for any of the shuffling reactions described herein.

It will further be appreciated that any of the above-described techniques suitable for enriching a library prior to shuffling can be used to screen the products generated by the methods of DNA shuffling.

The term “gene” is used herein broadly to refer to any nucleic acid segment or sequence associated with a biological function. In some embodiments of the invention, an initial screening of enzyme activities in a particular assay can be useful in identifying candidate nucleic acid sequences as starting materials. For example, high throughput screening can be used to screen enzymes for dioxygenase-type activities using aromatic acids as substrates. Dioxygenases typically transform indole-2-carboxylate and indole-3-carboxylate to colored products, including indigo (Eaton et al. (1995), J. Bacteriol. 177: 6983-6988). Genes encoding enzymes that show activity in the initial assay can then be recombined by the recursive techniques of the invention and screened further. The use of such initial screening for candidate enzymes against a desired target molecule or analog of the target molecule can be especially useful to generate enzymes that catalyze reactions of interest such as catabolism of man-made pollutants in bioremediation.

In general any bacterial strain can be used as a recipient of evolved genes. The choice of host depends on a number of factors, including the intended use of the spore system, environmental hardiness, presence of key intermediates, ease of genetic manipulation, and likelihood of promiscuous transfer of genetic information to other organisms.

If in vitro diversity-generating protocols are employed, the recombinant library is preferably introduced into the most appropriate bacterial strain before screening and/or selection. After introduction of the library of recombinant nucleic acid sequences, the bacterial strains are optionally propagated to allow expression of genes to occur.

Transformed or mutated strains are then screened and/or selected for characteristics of interest. Such a characteristic could be, for example, production of an enzyme showing enhanced degradation of a bioremediation substrate such as phenol. Screening for production of a desired compound, such as a therapeutic or prophylactic drug, can be accomplished by observing binding of cell products to a receptor or ligand, such as on a solid support or on a column. Such screening can additionally be accomplished by binding to antibodies, as in an ELISA. In some instances the screening process is preferably automated so as to allow screening of suitable numbers of colonies or cells. Selection may also be done by such techniques as, for example, growth on a toxic substrate to select for hosts having the ability to detoxify a substrate, growth on a new nutrient source to select for hosts having the ability to utilize that nutrient source, or competitive growth in culture based on ability to utilize a nutrient source. In some embodiments of the invention, screening can be accomplished by assaying reactivity with a reporter molecule reactive with a desired feature of a gene product. Thus, the library of nucleic acid sequences may be screened by an in vitro assay or transformed into an appropriate strain of bacteria, and each sequence is functionally tested using an appropriate assay. Spore systems of the present invention can be used to encapsulate or display libraries of polypeptides or nucleic acid molecules. In other words, spore systems that each individually encapsulate or display one or more distinct polypeptides or nucleic acid molecules, can collectively form a library that can be screened. Appropriate assays are capable of detecting significant changes in function relative to the original sequences employed in the mutagenizing process. An assay could evaluate immunoreactivity of the target antibody against a library of antigens.

By means of such screening and/or selections, the pool of cells remaining is enriched for recombinant genes conferring the desired phenotype (e.g., altered substrate specificity, altered biosynthetic ability, etc.). Nucleic acid sequences encoding the polypeptide of interest may be recovered from bacteria yielding the desired result in the assay. This nucleic acid sequence could be further mutagenized or recombined and tested functionally in vivo, ex vivo, or in vitro.

Thus, the nucleic acid sequences surviving a round of screening and/or selection can serve as the substrates for subsequent rounds of mutagenesis. Optionally, a subsequence of such nucleic acid sequence(s) can be isolated for more targeted mutagenesis. After each subsequent round of mutagenesis, genes conferring the desired phenotype are again selected, either essentially as before or using new selection or screening criteria. For example, the ability of bacteria to use a new substrate can be assayed in some instances by the ability to grow on a substrate as a nutrient source. In other circumstances such ability can be assayed by decreased toxicity of a substrate for a host cell, hence allowing the host to grow in the presence of that substrate.

Thus, polypeptides of interest may be identified in many ways. For example, polypeptides of interest may be identified using multi-tiered screening. In this method, a strain of bacteria, that may contain an introduced nucleic acid on an episome or plasmid or in its genome, is mutated and then put through a multistep selection process to collect a pool of mutant strains showing increased antigenicity relative to the unmutated display strain or the other mutated individuals in the display strain. After mutagenesis, the strain is put through a first step of spore panning selection, a second step of a competition assay to select the highest quantity of displayed antigen, and a third step of an in vivo assay in mouse. The resulting candidate bacteria represent a pool of the best candidates for a spore display system for a particular antigen. Further, the in vivo assay in mouse may be used to assay the efficacy of a vaccine by challenging a vaccinated mouse with the pathogen against which the vaccine was intended to protect.

One of skill in the art will be able to isolate and characterize the nucleic acid sequence encoding the polypeptide of interest from a strain identified by any of these assay methods.

Display on the Spore Coat

Methods of identifying proteins and mutations in said proteins that are useful in the practice of the present invention are provided. Identification of mutations and proteins useful in the present invention are accomplished by means of the techniques described herein. For example, a protein useful in the practice of the present invention provides a protein which when fused to another polypeptide provides localization of expression of the resulting fusion polypeptide to a location that enhances the usefulness of the polypeptide in a method or composition of the present invention. For example, a spore coat protein may be useful in providing for the display of a fusion protein on the surface of the spore. Multiple spore coat proteins constitute the matrix of the spore coat, and each may be useful in expression of the peptides or polypeptides of interest. For example, over 24 proteins are now known to take part in the formation of the outer protein layer of the spore coat of B. subtilis. Useful proteins may be identified using a screening process of the present invention. Sites in the Bacillus subtilis genome that display protein fusions on the surface of the spore may be identified by transposon mutagenesis of a small protein tag wherein transposon mutagenesis is performed on the structural coat protein genes (for example, Cot genes), regulatory genes involved in spore formation, or on the whole genome. A spore display system of the present invention may then be used to screen for expression of the protein tag, and candidate genes for construction of fusion proteins identified.

It is recognized that the polypeptide, protein, or peptide to be displayed on the spore may be attached to a spore protein at the N-terminus, the C-terminus, at the N-terminus to one coat protein and at the C-terminus to another coat protein, or attached to one coat protein at both the N-terminus and the C-terminus. For example, the fusion protein may be comprised of an N-terminal fusion to CotV and a C-terminal fusion to CotC. The two spore coat peptides would migrate to their appropriate positions on the spore coat, causing the polypeptide, protein, or peptide of interest to be displayed over a larger area of the spore and possibly causing a conformational distortion of the polypeptide of interest. The conformation change caused by such a distortion may expose antigenic regions of the polypeptide, protein, or peptide or may alter the enzymatic activity of the polypeptide, protein, or peptide. Thus this method could be used to screen for enzymes with a desired alteration in activity. Such alterations in activity may be assayed by any means known to one of skill in the art. In an additional example, the polypeptide to be displayed may be inserted into a single spore coat protein.

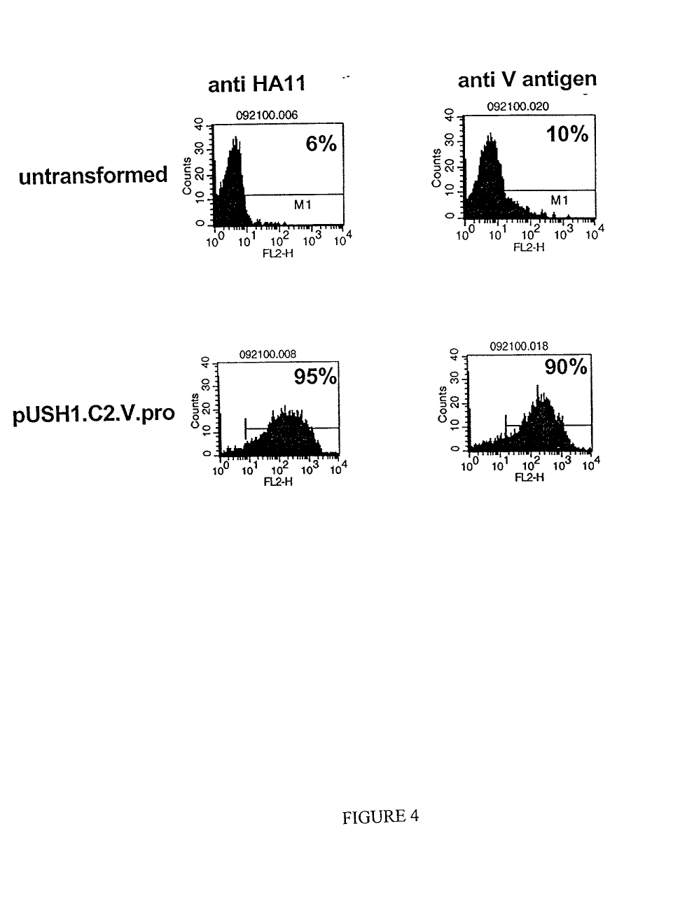

Different insertion sites within a single spore coat sequence alter the exposure characteristics of the fusion protein and may alter the conformation, enzymatic activity, or antigenic properties of the peptide encoded by the nucleotide sequence of interest. Optimal insertion points in the spore coat sequences of B. subtilis have been identified using a test epitope, HA11 (amino acid residues 31-43 of the polypeptide encoded by SEQ ID NO: 1), for which high affinity monoclonal antibodies are available. The nucleotide sequence encoding the HA11 epitope, or a nucleotide sequence of interest, is cloned into a construct where the nucleotide sequence is operably linked to a sequence encoding a spore coat protein such as CotA, CotB, CotC, CotE, CotG, CotV, CotX, CotY, CotW, and CotZ. The nucleotide sequence may be inserted at the N-terminus, C-terminus, or internal to the spore coat sequence. Methods for cloning and manipulating nucleotide sequences are well known in the art and reviewed elsewhere herein. After a fusion construct is created, it is transformed into B. subtilis or other sporulating bacteria or fungus using methods known in the art (Sambrook et al. (1989), Molecular Cloning: A Laboratory Manual (2^{nd} edition) (Cold Spring Harbor Laboratory Press, New York); Ausubel et al., eds. (1999), Current Protocols in Molecular Biology (John Wiley & Sons); and Sherman et al. (1982) Methods in Yeast Genetics, Cold Spring Harbor Laboratory). The spore coat proteins are released from the spore using a combination of heat, detergents, and denaturing agents. Localization of the HA 11 epitope to the spore coat is then determined by Western blot analysis (see FIG. 4). The level of HA11 in the spore coat indicates the quality of the insertion locus.

Transposon mutagenesis may also be used to identify insertion loci or insertion positions in bacterial genes. For example, in B. subtilis, an in vitro transposon vector containing the nucleotide sequence encoding the HA11 epitope was constructed. The HA11 epitope was inserted next to an antibiotic resistance gene, such as the tetR gene. A target plasmid containing the spore coat gene of interest, such as CotD, was constructed. The in vitro transposon construct was used to insert HA11 into the B. subtilis cotD gene. Mutant candidates were selected using an appropriate antibiotic. Since the transposition event may occur anywhere in the target plasmid, DNA was isolated from the antibiotic resistant clones and the target gene (i.e. cotD) was amplified by PCR. The size of the gene of interest and the transposon allowed for identification of the clones that have a transposon in the target gene (i.e. cotD). The antibiotic resistance gene was removed by cloning methods commonly known in the art, leaving the gene of interest in the target gene. The operably linked gene of interest and target gene cassette was cloned into an expression vector and transformed into B. subtilis. B. subtilis was then sporulated and the spore coat was assessed for display of the peptide of interest.

Insertion loci of interest include, but are not limited to, between amino acids 27 and 28, 47 and 48, 65 and 66, and 66 and 67 of CotC; amino acids 1 and 2, 16 and 17, 19 and 20, 48 and 49, 107 and 108, 163 and 164, 177 and 178, and after amino acid 195 of CotG; and amino acids 21 and 22, 44 and 45, and 50-51 of CotD.

Non-Viable Spores

The intact spore stage provides the advantage that antigens or polypeptides may be displayed or presented on the spore surface and that in some applications, the spore need not germinate to effectively provide the vaccine to the subject. That is, a spore system may comprise spores that are non-viable. For the spore display systems of the present invention, a completely non-germinating (i.e., non-viable) spore would be entirely acceptable for surface antigen display and enzyme display embodiments.

A non-viable spore is a spore that does not germinate. A non-viable spore may be produced by rendering nonfunctional at least one or more of the three cortex lytic genes (cwID, cwLJ, and sleB), each of which encodes a cortex lytic enzyme. By rendering one or more of these genes nonfunctional, the respective cortex lytic enzyme is not produced and the spore cannot germinate. Each such gene may be rendered nonfunctional by a variety of methods, including transposon mutagenesis and deletion recombination, thereby rendering the gene nonfunctional.

Additional methods used to decrease viability of the germination mutant (ger-) spores are known to those of skill. These include the following.

1. SASP (Spore-associated Acid Soluble Protein)-gene knock-outs: The α/β-type SASP proteins coat the spore DNA and protect it against heat and UV-irradiation. UV-treatment (280-300mn) of spores from a strain in which the two genes (sspA and sspB) that encode the major α/β-type SASP proteins has been knocked out has been shown to reduce the spore viability by greater than 1000-fold (see, Setlow, B. and Setlow, P. 1988, J. Bacteriol. 170:2858-9). This treatment reduces the ability of the spores to germinate to at least 1 in 10⁹.

2. Temperature sensitive (Ts) mutations: A temperature-sensitive mutation in a gene that is essential for bacterial growth and replication (such as a gene that encodes one or more subunits of a DNA polymerase or ribosomal RNA/protein) results in death of the cell at the non-permissive temperature (typically between 35 and 37C). A suitable Ts mutation is introduced into the ger-strain, and the resultant Ts ger-strain is maintained and sporulated at the permissive temperature (typically 30C) in the laboratory. Any Ts ger-spores that germinate in the immunization host will die because of the non-permissive temperature. Since Ts mutations typically give a 100-1000 fold reduction in viability, the Ts ger-spores typically give rise to viable spores at a frequency of 10⁻⁸ to

3. Endonuclease under the forespore-specific sigma G—dependent promoter: Another method to effect viability of germinated spores is to use an endonuclease that degrades the bacterial DNA. The sigG-dependent promoter is turned on only in the forespore compartment late in sporulation, and the endonuclease under the control of such a promoter is synthesized only in the spores. Furthermore, it is synthesized at a time when the spore DNA is protected by the SASP proteins. Thus, the endonuclease does not interfere with sporulation. However, during germination after the SASP proteins are degraded by the GPR germination protease, the endonuclease gains access to and degrades the DNA; thus killing the germinated spore. This sigG-controlled endonuclease can be introduced into a WT spore or into ger-strain. The viability of spores from that strain is typically less than 1 in 10⁹. A variety of endonucleases can be used for this purpose. For example, the endonuclease genes yosQ, ywqL, ywJD, yqfS from Bacillus subtilis and endA, hsdR from E. coli encode endonucleases and can be used in this procedure.

4. Treatment by irradiation. Exposing the spores to gamma radiation (such as from a Co₆₀ source) reduces their viability by damaging the DNA. Currently, such irradiation is used for sterilization of disposable medical equipment as well as some food products, and similar standardized procedures could be applied to the genetically modified spores to further reduce their viability. Irradiation does not damage a molecule of interest displayed on, contained in, or associated with a spore; thus, such treatment is useful for generating non-viable spores.

A non-viable spore system comprises a non-viable spore and a nucleic acid molecule, polypeptide, protein, or peptide of interest. The nucleic acid molecule, polypeptide, protein, or peptide may be displayed on the surface of the spore, contained within the spore, or incorporated into the coat protein of the spore.

Non-viable spore systems are useful in all uses described herein. For example, non-viable spore systems find use in applications where spore systems are useful but spore viability is not required or desired. For example, a non-viable spore system may comprise a spore associated with a nucleic acid, polypeptide, protein, or peptide which produces or invokes an immunomodulatory response or associated with an antibody (Ab), DNA binding protein(s), or ligand-specific binding protein(s) and may be useful in a wide variety of therapeutic, prophylactic, medicinal, and/or pharmaceutical applications. Non-viable spore systems may also comprise a spore associated with an enzyme or biocatalytic agent and may be useful in industrial, biocatalytic, and/or agricultural applications. Enzymes displayed on the surface of a non-viable spore, as in the situation with other immobilizing technologies, are often significantly more stable and active. Germination of the spore involves loss of the spore coat and thus loss of peptides, polypeptides, proteins, carbohydrates, or nucleotide sequences of interest displayed on the spore surface. Further, germination of a viable spore may result in release of contaminants that are not desirable in an application. Thus, the present invention provides methods for enhancing enzymatic activities so as to increase the longevity or activity of the enzymatic activity. Such enhanced enzymatic activities have use in applications where increased longevity or activity of an enzyme is beneficial. For example, such enhanced enzymatic activities find use in fermentation and bioreactor applications. Non-viable and viable spores that display or contain peptides or nucleic acids of interest are also useful as a delivery platform(s) or vehicles for the delivery of peptides, polypeptides, proteins, carbohydrates, and/or nucleic acid molecules.

Preparation and Administration of Spore Systems as Therapeutic and Prophylactic Agents, and Vaccines; Medical Applications

The peptides, polypeptides, proteins, and nucleic acids of interest may have the biological activity of modulating an immune response. Thus, in one embodiment, a spore display system expressing a polypeptide, protein, or antigen of interest is used to modulate an immune response in vivo in a vertebrate or invertebrate, ex vivo, or in vitro. For example, in one embodiment, bacterial cells are transformed with a DNA sequence encoding an antigen, and the bacterial cells are induced to sporulate. The spores display the antigen or polypeptide as part of the spore coat upon or after sporulation. In an additional embodiment, a spore display system displaying a nucleotide sequence of interest on the spore surface is used to modulate an immune response in vivo in a vertebrate or invertebrate, ex vivo, or in vitro.

By “modulation” of an immune response of a subject is intended that the immune response of the subject is altered. Thus, for example, by “modulation” is intended that the immune response of a subject is stimulated, invoked, decreased, increased, enhanced, or otherwise altered. For example, the immunological response may be skewed or shifted from Th1 to Th2 or vice versa to optimize protection and reduce unwanted side effects of the immunological response. As used herein, an “immunomodulatory agent” is an agent that modulates an immune response. Such an immunomodulatory agent has immunomodulatory activity. A modulated immune response differs from the immune response of an untreated subject by 0.1%, 0.5%, 1%, 5%, 10%, 20%, 30%, 40%, 50%, 60%, 70%, 80%, 90%, 100%, or more. Modulation of the immune response by bacterial strains derived from the spore system or spore display system can be assessed by means known to those skilled in the art. Generation, selection, and assessment of the antigenicity or other desired activity of a polypeptide or nucleotide sequence, including efficacy as a vaccine, may be accomplished by such methods known to those in the art, including the particular methods described below, which are included by way of illustration and not of limitation. This and other techniques useful in the practice of the present invention and known to those skilled in the art are described in method reference manuals, such as Sambrook et al. (1989), Molecular Cloning: A Laboratory Manual (2^{nd} edition) (Cold Spring Harbor Laboratory Press, New York); Current Protocols in Immunology, John Colligan et al., eds., Vols. I-IV (John Wiley & Sons, Inc., N.Y., 1991 and 2001 Supplement) (hereinafter “Colligan”); Rapley, R. and Walker, J. M. eds., Molecular Biomethods Handbook (Humana Press, Inc. 1998) (hereinafter “Rapley and Walker”)and Ausubel et al., eds. (1999), Current Protocols in Molecular Biology (John Wiley & Sons). See also, Raz et al. (1994) Proc. Natl. Acad. Sci. 91:9519-9523 and Eisenbraun et al. (1993) DNA & Cell Biol. 12:791-797.

For example, the methods of the invention include methods for modulating immune responses in a subject using the spore systems described herein and, in other aspects, allow identification and cloning of immunomodulatory molecules and antigens suitable for use as immunomodulatory agents or vaccines against numerous diseases, disorders, and conditions. The spore system of the invention may, e.g., display at least one antigen or immunomodulatory molecule, or display or bind at least one nucleic acid, that allows the use of a spore system of the invention as a vaccine or immunomodulatory agent for therapeutic and/or prophylactic applications.

In one aspect, disease-associated antigens are incorporated into spores as, e.g., using one of the display, presentation, or attachment formats described above so as to display, present, bind or express the antigen on the surface of a spore. The antigen can also be expressed on the spore surface by, e.g., incorporating a DNA plasmid vector comprising a nucleotide sequence encoding the antigen into the spore and facilitating expression of the antigen on the spore surface. The invention also provides methods using such spore systems and vaccines comprising such spore systems to treat and prevent diseases and conditions associated with the antigens.

In one aspect, a spore system of the invention may function as a therapeutic, prophylactic, or immunomodulatory agent or vaccine against a disease or disease-inducing pathogen, including but not limited to, Yersinia pestis, Staphylococcus aureus, Streptococcus pyogenes, viral encephalitis, Herpes simplex virus Type 1, Herpes simplex virus Type 2, Varicella-zoster virus (Herpes zoster), cytomegalovirus, poliomyelitis, rabies, cancer, typhoid, parasites (including, e.g., parasites associated with malaria, African sleeping sickness, Giardia), typhus, anthrax, foot and mouth disease, HIV, pertussis, diphtheria, Ebola, hemorrhagic fevers, influenza, smallpox, cholera, dengue fever, measles, mumps, German measles, chicken pox, hepatitis A, hepatitis B, hepatitis C, Alzheimer's, human papillomavirus, meningitis, mononucleosis, Lyme disease, tetanus, Rocky Mountain spotted fever, Salmonella, Yellow fever, pneumonia, Mycobacterium tuberculosis, Respiratory Syncytial Virus, Creutzfield-Jacob Disease, Chlamydia trachomatis, syphilis, Listeria monocytogenes, Gonorrhea, Parvovirus, Paramyxoviridae diseases, Coxsackievirus, Rhinovirus, hantavirus, Japanese encephalitis, Eastern equine encephalitis, Western equine encephalitis, tick-borne encephalitis, West Nile Encephalitis, and Legionella pneumophila, bacterial enterotoxigenic strains of E. coli (e.g., heat-labile toxin from E. coli), and salmonella toxin, shigella toxin and campylobacter toxin. Spore systems comprising antigens or antigenic peptides associated with such diseases or toxins (e.g., having antigens expressed or displayed on the spore surface) can be prepared in any of the formats described herein and used in the therapeutic or prophylactic methods described herein.

For example, in one aspect, a spore system is designed to display a HIV antigen (e.g., Gp120, gp41, GAGcp24) by uptake and expression by the spore of a plasmid vector encoding such antigen, or display such antigen as a fusion protein with a spore coat protein, or display such antigen by chemical coupling of a biotinylated HIV antigen to the spore surface via an biotin-avidin linkage. The spore system is then used as a vaccine or immunomodulatory agent in methods for preventing against or therapeutically treating HIV infection.

The disease-associated antigens include, but are not limited to, cancer antigens, such as tumor-associated antigens expressed on cancer cells, antigens associated with autoimmunity disorders, antigens associated with inflammatory conditions, antigens associated with allergic reactions, antigens associated with infectious agents, and autoantigens that play a role in induction of autoimmune diseases.

Examples of cancer antigens that can be used with spore systems and methods of the invention include, but are not limited to, Among the tumor-specific antigens that can be used in the antigen shuffling methods of the invention are: bullous pemphigoid antigen 2, prostate mucin antigen (PMA) (Beckett and Wright (1995) Int. J. Cancer 62: 703-710), tumor associated Thomsen-Friedenreich antigen (Dahlenborg et al. (1997) Int. J. Cancer 70: 63-71), prostate-specific antigen (PSA) (Dannull and Belldegrun (1997) Br. J. Urol. 1: 97-103), EpCam/KSA antigen, luminal epithelial antigen (LEA.135) of breast carcinoma and bladder transitional cell carcinoma (TCC) (Jones et al. (1997) Anticancer Res. 17: 685-687), cancer-associated serum antigen (CASA) and cancer antigen 125 (CA 125) (Kierkegaard et al. (1995) Gynecol. Oncol. 59: 251-254), the epithelial glycoprotein 40 (EGP40) (Kievit et al. (1997) Int. J. Cancer 71: 237-245), squamous cell carcinoma antigen (SCC) (Lozza et al. (1997) Anticancer Res. 17: 525-529), cathepsin E (Mota et al. (1997) Am. J. Pathol. 150: 1223-1229), tyrosinase in melanoma (Fishman et al. (1997) Cancer 79: 1461-1464), cell nuclear antigen (PCNA) of cerebral cavernomas (Notelet et al. (1997) Surg. Neurol. 47: 364-370), DF3/MUC1 breast cancer antigen (Apostolopoulos et al. (1996) Immunol. Cell. Biol. 74: 457-464; Pandey et al. (1995) Cancer Res. 55: 4000-4003), carcinoembryonic antigen (Paone et al. (1996) J. Cancer Res. Clin. Oncol. 122: 499-503; Schlom et al. (1996) Breast Cancer Res. Treat. 38: 27-39), tumor-associated antigen CA 19-9 (Tolliver and O'Brien (1997) South Med. J. 90: 89-90; Tsuruta et al. (1997) Urol. Int. 58: 20-24), human melanoma antigens MART-1/Melan-A27-35 and gp100 (Kawakami and Rosenberg (1997) Int. Rev. Immunol. 14: 173-192; Zajac et al. (1997) Int. J. Cancer 71: 491-496), the T and Tn pancarcinoma (CA) glycopeptide epitopes (Springer (1995) Crit. Rev. Oncog. 6: 57-85), a 35 kD tumor-associated autoantigen in papillary thyroid carcinoma (Lucas et al. (1996) Anticancer Res. 16: 2493-2496), KH-1 adenocarcinoma antigen (Deshpande and Danishefsky (1997) Nature 387: 164-166), the A60 mycobacterial antigen (Maes et al. (1996) J. Cancer Res. Clin. Oncol. 122: 296-300), heat shock proteins (HSPs) (Blachere and Srivastava (1995) Semin. Cancer Biol. 6: 349-355), and MAGE, tyrosinase, melan-A and gp75 and mutant oncogene products (e.g., p53, ras, and HER-2/neu (Bueler and Mulligan (1996) Mol. Med. 2: 545-555; Lewis and Houghton (1995) Semin. Cancer Biol. 6: 321-327; Theobald et al. (1995) Proc. Nat'l. Acad. Sci. USA 92: 11993-11997).

In one aspect, the invention provides spore systems displaying at least one rotavirus capsid protein VP4, VP6, or VP7. Such spore systems are useful in methods for inducing an immune response against a VP4, VP6, or VP7 rotavirus, respectively.

Additional viral antigens that can be used with spore systems of the invention, methods for modulating immune responses against diseases and disorders associated with such antigens, and vaccines comprising spore systems, include, but are not limited to, hepatitis B capsid protein, hepatitis C capsid protein, hepatitis A capsid protein, Norwalk diarrheal virus capsid protein, influenza A virus N2 neuraminidase (Kilbourne et al. (1995) Vaccine 13: 1799-1803); Dengue virus envelope (E) and premembrane (prM) antigens (Feighny et al. (1994) Am. J. Trop. Med. Hyg. 50: 322-328; Putnak et al. (1996) Am. J. Trop. Med. Hyg. 55: 504-10); HIV antigens Gag, Pol, Vif and Nef (Vogt et al. (1995) Vaccine 13: 202-208); HIV antigens gp120 and gp160 (Achour et al. (1995) Cell. Mol. Biol. 41:395-400; Hone et al. (1994) Dev. Biol. Stand. 82: 159-162); gp41 epitope of human immunodeficiency virus (Eckhart et al. (1996) J. Gen. Virol. 77:2001-2008); rotavirus antigen VP4 (Mattion et al. (1995) J. Virol. 69:5132-5137); the rotavirus protein VP7 or VP7sc (Emslie et al. (1995) J. Virol. 69: 1747-1754; Xu et al. (1995) J. Gen. Virol. 76: 1971-1980; Chen et al. (1998) Journal of Virology Vol 72:7; pp 5757-5761); herpes simplex virus (HSV) glycoproteins gB, gC, gD, gE, gG, gH, and gI (Fleck et al. (1994) Med. Microbiol. Immunol. (Berl) 183: 87-94 [Mattion, 1995]; Ghiasi et al. (1995) Invest. Ophthalmol. Vis. Sci. 36: 1352-1360; McLean et al. (1994) J. Infect. Dis. 170: 1100-1109); immediate-early protein ICP47 of herpes simplex virus-type 1 (HSV-1) (Banks et al. (1994) Virology 200:236-245); immediate-early (IE) proteins ICP27, ICPO, and ICP4 of herpes simplex virus (Manickan et al. (1995) J. Virol. 69: 4711-4716); influenza virus nucleoprotein and hemagglutinin (Deck et al. (1997) Vaccine 15: 71-78; Fu et al. (1997) J. Virol. 71: 2715-2721); B19 parvovirus capsid proteins VPI (Kawase et al. (1995) Virology 211: 359-366) or VP2 (Brown et al. (1994) Virology 198: 477-488); Hepatitis B virus core and e antigen (Schodel et al. (1996) Intervirology 39: 104-106); hepatitis B surface antigen (Shiau and Murray (1997) J. Med. Virol. 51: 159-166); hepatitis B surface antigen fused to the core antigen of the virus (Id.); Hepatitis B virus core-preS2 particles (Nemeckova et al. (1996) Acta Virol. 40: 273-279); HBV preS2-S protein (Kutinova et al. (1996) Vaccine 14: 1045-1052); VZV glycoprotein I (Kutinova et al. (1996) Vaccine 14: 1045-1052); rabies virus glycoproteins (Xiang et al. (1994) Virology 199: 132-140; Xuan et al. (1995) Virus Res. 36: 151-161) or ribonucleocapsid (Hooper et al. (1994) Proc. Nat'l. Acad. Sci. USA 91: 10908-10912); human cytomegalovirus (HCMV) glycoprotein B (UL55) (Britt et al. (1995) J. Infect. Dis. 171: 18-25); the hepatitis C virus (HCV) nucleocapsid protein in a secreted or a nonsecreted form, or as a fusion protein with the middle (pre-S2 and S) or major (S) surface antigens of hepatitis B virus (HBV) (Inchauspe et al. (1997) DNA Cell Biol. 16: 185-195; Major et al. (1995) J. Virol. 69: 5798-5805); the hepatitis C virus antigens: the core protein (pC); E1 (pE1) and E2 (pE2) alone or as fusion proteins (Saito et al. (1997) Gastroenterology 112: 1321-1330); the gene encoding respiratory syncytial virus fusion protein (PFP-2) (Falsey and Walsh (1996) Vaccine 14: 1214-1218; Piedra et al. (1996) Pediatr. Infect. Dis. J. 15: 23-31); the VP6 and VP7 genes of rotaviruses (Choi et al. (1997) Virology 232: 129-138; Jin et al. (1996) Arch. Virol. 141: 2057-2076); the E1, E2, E3, E4, E5, E6 and E7 proteins of human papillomavirus (Brown et al. (1994) Virology 201: 46-54; Dillner et al. (1995) Cancer Detect. Prev. 19:381-393; Krul et al. (1996) Cancer Immunol. Immunother. 43: 44-48; Nakagawa et al. (1997) J. Infect. Dis. 175: 927-931); a human T-lymphotropic virus type I gag protein (Porter et al. (1995) J. Med. Virol. 45: 469-474); Epstein-Barr virus (EBV) gp340 (Mackett et al. (1996) J. Med. Virol. 50:263-271); the Epstein-Barr virus (EBV) latent membrane protein LMP2 (Lee et al. (1996) Eur. J. Immunol. 26: 1875-1883); Epstein-Barr virus nuclear antigens 1 and 2 (Chen and Cooper (1996) J. Virol. 70: 4849-4853; Khanna et al. (1995) Virology 214: 633-637); the measles virus nucleoprotein (N) (Fooks et al. (1995) Virology 210: 456-465); and cytomegalovirus glycoprotein gB (Marshall et al. (1994) J. Med. Virol. 43: 77-83) or glycoprotein gH (Rasmussen et al. (1994) J. Infect. Dis. 170: 673-677).

Examples of medical conditions and/or diseases where down-regulation or decreased immune response is desirable include, but are not limited to, allergy, asthma, autoimmune diseases (e.g., rheumatoid arthritis, SLE, diabetes mellitus, myasthenia gravis, reactive arthritis, ankylosing spondylitis, and multiple sclerosis), septic shock, organ transplantation, and inflammatory conditions, including IBD, psoriasis, pancreatitis, and various immunodeficiencies.

Autoimmune diseases and inflammatory conditions are often characterized by an accumulation of inflammatory cells, such as lymphocytes, macrophages, and neutrophils, at the sites of inflammation. Altered cytokine production levels are often observed, with increased levels of cytokine production. Several autoimmune diseases, including diabetes and rheumatoid arthritis, are linked to certain MHC haplotypes. Other autoimmune-type disorders, such as reactive arthritis, have been shown to be triggered by bacteria such as Yersinia and Shigella, and evidence suggests that several other autoimmune diseases, such as diabetes, multiple sclerosis, rheumatoid arthritis, may also be initiated by viral or bacterial infections in genetically susceptible individuals. Examples of antigens for use in spore systems and methods of the invention to treat autoimmune diseases, inflammatory conditions, and other immunodeficiency-associated conditions are provided in Punnonen et al. (1999) WO 99/41369; Punnonen et al. (1999) WO 99/41383; Punnonen et al. (1999) WO 99/41368; and Punnonen et al. (1999) WO 99/41402), each of which is incorporated herein by reference for all purposes.

For treatment or prevention of such diseases or conditions, spore systems comprising one or more polypeptides, proteins, peptides, or nucleic acids capable of reducing or suppressing an immune response (e.g., antigens specific for or associated with a disease), such as T cell proliferation or activation, can be administered according to the methods described herein.

For example, in another aspect, the invention provides spore systems and vaccines for treating allergies, and prophylactic and therapeutic treatment methods utilizing such spore systems and vaccines. Antigens of allergens can be incorporated into spore systems as, e.g., using one of the display, presentation, or attachment formats described above so as to display, present, bind or express the antigen on the surface of a spore. The antigen can also be expressed on the spore surface by, e.g., incorporating a DNA plasmid vector comprising a nucleotide sequence encoding the antigen into the spore and facilitating expression of the antigen on the spore surface.

Examples of allergies that can be treated using methods and spore systems of the invention include, but are not limited to, allergies against house dust mite, grass pollen, birch pollen, ragweed pollen, hazel pollen, cockroach, rice, olive tree pollen, fungi, mustard, bee venom. Antigens of interest include those of animals, including the mite (e.g., Dermatophagoides pteronyssinus, Dermatophagoides farinae, Blomia tropicalis), such as the allergens der p1 (Scobie et al. (1994) Biochem. Soc. Trans. 22: 448S; Yssel et al. (1992) J. Immunol. 148: 738-745), der p2 (Chua et al. (1996) Clin. Exp. Allergy 26: 829-837), der p3 (Smith and Thomas (1996) Clin. Exp. Allergy 26: 571-579), der p5, der p V (Lin et al. (1994) J. Allergy Clin. Immunol. 94: 989-996), der p6 (Bennett and Thomas (1996) Clin. Exp. Allergy 26: 1150-1154), der p 7 (Shen et al. (1995) Clin. Exp. Allergy 25: 416-422), der f2 (Yuuki et al. (1997) Int. Arch. Allergy Immunol. 112: 44-48), der f3 (Nishiyama et al. (1995) FEBS Lett. 377: 62-66), der f7 (Shen et al. (1995) Clin. Exp. Allergy 25: 1000-1006); Eur m 1 and Eur m 2; Mag 3 (Fujikawa et al. (1996) Mol. Immunol. 33: 311-319). Also of interest as antigens for use with the invention are the house dust mite allergens Tyr p2 (Eriksson et al. (1998) Eur. J. Biochem. 251: 443-447), Lep d1 (Schmidt et al. (1995) FEBS Lett. 370: 11-14), and glutathione S-transferase (O'Neill et al. (1995) Immunol Lett. 48: 103-107); the 25,589 Da, 219 amino acid polypeptide with homology with glutathione S-transferases (O'Neill et al. (1994) Biochim. Biophys. Acta. 1219: 521-528); Blo t 5 (Arruda et al. (1995) Int. Arch. Allergy Immunol. 107: 456-457); bee venom phospholipase A2 (Carballido et al. (1994) J. Allergy Clin. Immunol. 93: 758-767; Jutel et al. (1995) J. Immunol. 154: 4187-4194); bovine dermal/dander antigens BDA 11 (Rautiainen et al. (1995) J. Invest. Dermatol. 105: 660-663) and BDA20 (Mantyjarvi et al. (1996) J. Allergy Clin. Immunol. 97: 1297-1303); the major horse allergen Equ c1 (Gregoire et al. (1996) J. Biol. Chem. 271: 32951-32959); Jumper ant M. pilosula allergen Myr p I and its homologous allergenic polypeptides Myr p2 (Donovan et al. (1996) Biochem. Mol. Biol. Int. 39: 877-885); 1-13, 14, 16 kD allergens of the mite Blomia tropicalis (Caraballo et al. (1996) J. Allergy Clin. Immunol. 98: 573-579); the cockroach allergens Bla g Bd90K (Helm et al. (1996) J. Allergy Clin. Immunol. 98: 172-80) and Bla g 2 (Arruda et al. (1995) J. Biol. Chem. 270: 19563-19568); the cockroach Cr-PI allergens (Wu et al. (1996) J. Biol. Chem. 271: 17937-17943); fire ant venom allergen, Sol i 2 (Schmidt et al. (1996) J. Allergy Clin. Immunol. 98: 82-88); the insect Chironomus thummi major allergen Chi t 1-9 (Kipp et al. (1996) Int. Arch. Allergy Immunol. 110: 348-353); dog allergen Can f 1 or cat allergen Fel d 1 (Ingram et al. (1995) J. Allergy Clin. Immunol. 96: 449-456); albumin, derived, for example, from horse, dog or cat (Goubran Botros et al. (1996) Immunology 88: 340-347); deer allergens with the molecular mass of 22 kD, 25 kD or 60 kD (Spitzauer et al. (1997) Clin. Exp. Allergy 27: 196-200); and the 20 kd major allergen of cow (Ylonen et al. (1994) J. Allergy Clin. Immunol. 93: 851-858).

The invention also includes spore systems, vaccines, and prophylactic and therapeutic methods for treating pollen and grass allergens. Such spore systems and vaccines are prepared using pollen and grass allergen antigenic polypeptides or DNA encoding such polypeptides, including, for example, Johnson grass, Hor v9 (Astwood and Hill (1996) Gene 182: 53-62, Lig v1 (Batanero et al. (1996) Clin. Exp. Allergy 26: 1401-1410); Lol p 1 (Muller et al. (1996) Int. Arch. Allergy Immunol. 109: 352-355), Lol p II (Tamborini et al. (1995) Mol. Immunol. 32: 505-513), Lol pVA, Lol pVB (Ong et al. (1995) Mol. Immunol. 32: 295-302), Lol p 9 (Blaher et al. (1996) J. Allergy Clin. Immunol. 98: 124-132); Par J I (Costa et al. (1994) FEBS Lett. 341: 182-186; Sallusto et al. (1996) .J Allergy Clin. Immunol. 97: 627-637), Par j 2.0101 (Duro et al. (1996) FEBS Lett. 399: 295-298); Bet v1 (Faber et al. (1996) J. Biol. Chem. 271: 19243-19250), Bet v2 (Rihs et al. (1994) Int. Arch. Allergy Immunol. 105: 190-194); Dac g3 (Guerin-Marchand et al. (1996) Mol. Immunol. 33: 797-806); Ph1 p 1 (Petersen et al. (1995) J. Allergy Clin. Immunol. 95: 987-994), Ph1 p 5 (Muller et al. (1996) Int. Arch. Allergy Immunol. 109: 352-355), Ph1 p 6 (Petersen et al. (1995) Int. Arch. Allergy Immunol. 108: 55-59); Cry j I (Sone et al. (1994) Biochem. Biophys. Res. Commun. 199: 619-625), Cry j II (Namba et al. (1994) FEBS Lett. 353: 124-128); Cor a 1 (Schenk et al. (1994) Eur. J. Biochem. 224: 717-722); cyn d1 (Smith et al. (1996) J. Allergy Clin. Immunol. 98: 331-343), cyn d7 (Suphioglu et al. (1997) FEBS Lett. 402: 167-172); Pha a 1 and isoforms of Pha a 5 (Suphioglu and Singh (1995) Clin. Exp. Allergy 25: 853-865); Cha o 1 (Suzuki et al. (1996) Mol. Immunol. 33: 451-460); profilin derived, e.g, from timothy grass or birch pollen (Valenta et al. (1994) Biochem. Biophys. Res. Commun. 199: 106-118); P0149 (Wu et al. (1996) Plant Mol. Biol. 32:1037-1042); Ory s1 (Xu et al. (1995) Gene 164: 255-259); and Amb a V and Amb t 5 (Kim et al. (1996) Mol. Immunol. 33: 873-880; Zhu et al. (1995) J. Immunol. 155: 5064-5073).

Therapeutic and prophylactic agents and vaccines against food allergens and treatment methods for food allergies can also be developed using spore systems and the methods of the invention. Suitable antigens for development of such vaccines include, for example, profilin (Rihs et al. (1994) Int. Arch. Allergy Immunol. 105: 190-194); rice allergenic cDNAs belonging to the alpha-amylase/trypsin inhibitor gene family (Alvarez et al. (1995) Biochim Biophys Acta 1251: 201-204); the main olive allergen, Ole e I (Lombardero et al. (1994) Clin Exp Allergy 24: 765-770); Sin a 1, the major allergen from mustard (Gonzalez De La Pena et al. (1996) Eur J. Biochem. 237: 827-832); parvalbumin, the major allergen of salmon (Lindstrom et al. (1996) Scand. J. Immunol. 44: 335-344); apple allergens, such as the major allergen Mal d 1 (Vanek-Krebitz et al. (1995) Biochem. Biophys. Res. Commun. 214: 538-551); and peanut allergens, such as Ara h I (Burks et al. (1995) J. Clin. Invest. 96: 1715-1721).

The invention also includes spore systems, vaccines, and prophylactic and therapeutic treatment methods effective against allergies to fungi and methods of using such vaccines. Fungal allergens useful in these vaccines include, but are not limited to, the allergen, Cla h III, of Cladosporium herbarum (Zhang et al. (1995) J. Immunol. 154: 710-717); the allergen Psi c 2, a fungal cyclophilin, from the basidiomycete Psilocybe cubensis (Horner et al. (1995) Int. Arch. Allergy Immunol. 107: 298-300); hsp 70 cloned from a cDNA library of Cladosporium herbarum (Zhang et al. (1996) Clin Exp Allergy 26: 88-95); the 68 kD allergen of Penicillium notatum (Shen et al. (1995) Clin. Exp. Allergy 26: 350-356); aldehyde dehydrogenase (ALDH) (Achatz et al. (1995) Mol Immunol. 32: 213-227); enolase (Achatz et al. (1995) Mol. Immunol. 32: 213-227); YCP4 (Id.); acidic ribosomal protein P2 (Id.).

Other allergens that can be used in the spore systems and methods of the invention include latex allergens, such as a major allergen (Hev b 5) from natural rubber latex (Akasawa et al. (1996) J. Biol. Chem. 271: 25389-25393; Slater et al. (1996) J. Biol. Chem. 271: 25394-25399).

Additional examples of antigens, auto-antigens, co-stimulatory molecules, and immunomodulatory molecules for use in spore systems and methods of the invention are set forth in Punnonen et al. (1999) WO 99/41369; Punnonen et al. (1999) WO 99/41383; Punnonen et al. (1999) WO 99/41368; and Punnonen et al. (1999) WO 99/41402). Other useful antigens have been described in the literature or can be discovered using genomics approaches.

In an embodiment of the invention, the spore-system may function as a broad-spectrum vaccine. By “broad-spectrum vaccine” is intended a vaccine that confers resistance to more than one disease or pathogen. A broad-spectrum vaccine may be comprised of a single antigen to which the immune system of the host organism generates antibodies directed towards more than one disease or pathogen. Such an antigen may be identified by DNA shuffling techniques, described elsewhere herein. A suitable starting material for shuffling would be polypeptide encoding nucleic acid sequences of a pathogen, preferably antigen-encoding nucleic acid sequences. In this manner, spore systems could be used to develop broadly cross-reactive antigens for use in developing vaccines.

The broad-spectrum or “multivalent vaccine” may be comprised of a spore system displaying more than one peptide or polypeptide with antigenic properties. The peptides or polypeptides may be derived from different pathogens, different strains of a pathogen, or disease conditions such that the host organism responds by generating antisera to more than one pathogen or disease condition.

An additional embodiment of the invention is a multi-component vaccine. By “multi-component vaccine” is intended a spore system that displays an antigen specific for a disease and at least one additional agent that further increases the immune response of the host organism to the antigen. The at least one additional agent may be an adjuvant, immunomodulatory agent, cytokine, co-stimulatory molecule, a detector molecule, a therapeutic or prophylactic agent or drug, nucleic acid, polypeptide, protein, peptide, antigen, etc., or one or more combinations of any such agents.

The invention also provides a spore system comprising one or more combinations of any one of the following components: nucleic acids, polypeptides, proteins, peptides, antigens, co-stimulatory agents, immunomodulatory molecules, adjuvants, cytokines, any of the biotinylated molecules bound to the spore surface via streptavidin or avidin as described above, or other molecules of interest. Such components can be, e.g., displayed on, presented on, bound or attached to the spore surface, encapsulated or contained with the spore, associated with the spore, carried or held by the spore, or coated onto the spore surface. Such combinations of multiple components and different components are especially useful in methods of modulating immune responses. For example, the use of an antigen and co-stimulatory molecule or cytokine in conjunction with one another can augment the immunostimulatory response, since both types of molecules are integral to responses. Similarly, the use of an adjuvant with an antigen and adjuvant can dramatically increase the immunostimulatory effectiveness of the antigen. Spore systems can be made to comprise selected combinations of such molecules dependent upon the specific application and treatment protocol. Methods of modulating immune response in a subject by administering such spore systems or compositions thereof in an amount sufficient to modulate the response are also included.

A spore system of the invention is also useful in therapeutic or prophylactic treatment methods for treating or preventing any of the above-mentioned diseases and disorders when administered to a subject as, e.g., a spore expressing a therapeutic or prophylactic polypeptide or a gene-based therapeutic or prophylactic polypeptide (i.e., polypeptide product expressed by a gene), wherein such spores are delivered alone or co-administered simultaneously or subsequently with one or more of an antigen, another co-stimulatory molecule, or adjuvant. A spore system of the invention is also useful for treating or preventing any of the above-mentioned diseases and disorders when administered to a subject as a genetic vaccine (e.g., DNA vaccine) comprising a spore comprising at least one therapeutic or prophylactic polypeptide-encoding polynucleotide or expression vector encoding at least one such polypeptide. If desired, such a genetic vaccine can be co-administered with a second expression vector encoding at least one additional immunomodulatory agent, such as an antigen, co-stimulatory molecule, and/or adjuvant. Alternatively, if desired, the genetic vaccine comprises a spore system comprising at least one single expression vector that encodes at least one therapeutic or prophylactic polypeptide-encoding polynucleotide and at least one of an antigen, co-stimulatory molecule, and/or adjuvant. In this format, the polynucleotide is co-expressed with at least one antigen, co-stimulatory molecule, and/or adjuvant.

Antigens or immunomodulatory or immunogenic polypeptides, peptides, or proteins of interest, including those described herein, may be identified by screening candidate sequences, as described more fully elsewhere herein. Candidate nucleotide sequences may be identified, selected, and/or isolated from libraries, which may contain mutagenized, randomized, or synthesized nucleic acid sequences. Such mutagenesis or randomization may be accomplished by a number of methods, as described more fully below. Libraries are transformed into an appropriate bacterial strain; such antigen libraries may be created by “shuffling,” by more conventional chemical or radiation mutagenesis, by molecular methods of mutagenesis or by some other method or means. Alternatively, mutagenesis of a library may be accomplished by transforming a bacterial strain with a library and mutagenizing the library-containing strain. A library containing recombinant nucleotide sequences encoding recombinant polypeptides is then transformed into an appropriate strain of bacteria, and each sequence is thereby tested against an assay which is capable of detecting immunoreactivity of the target antibody against the library of newly created antigens. Bacterial strains displaying antigens that are recognized by particular antibodies may be identified with the methods and compositions of the present invention. Once a strain of interest is thereby identified, the nucleic acid encoding the antigen of interest can be isolated from that particular strain and characterized by determination of the nucleic acid sequence and testing for antigenicity in vivo, ex vivo, or in vitro, using methods known to those of skill in the art and those described herein.

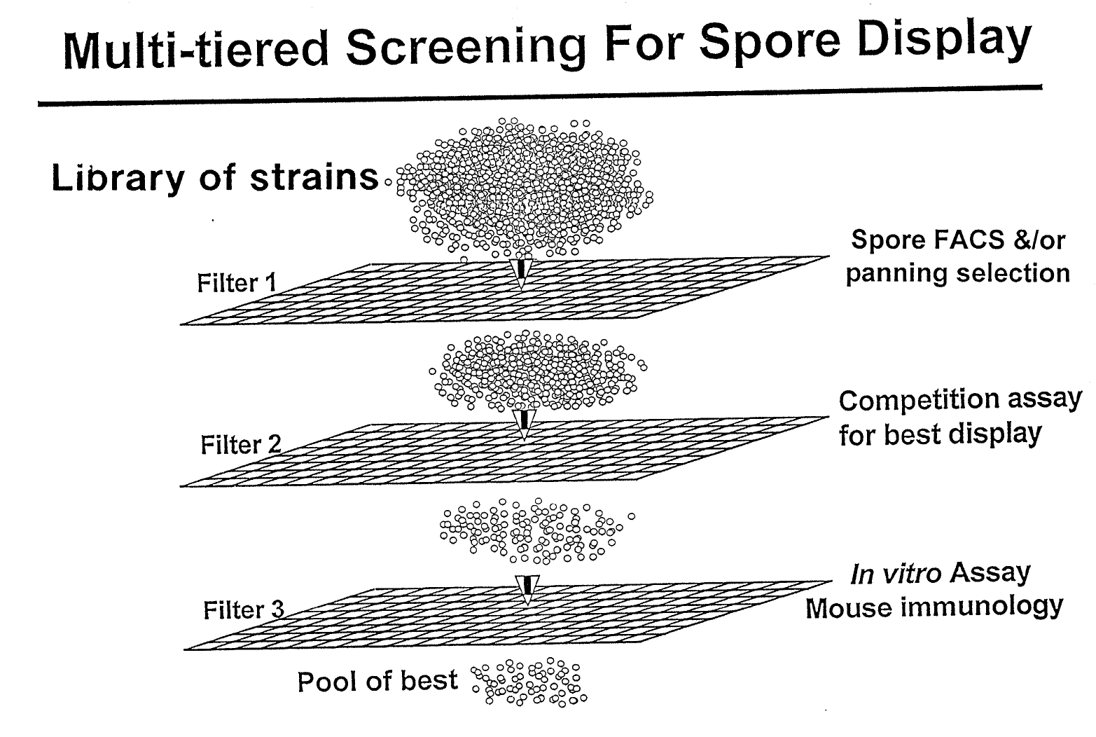

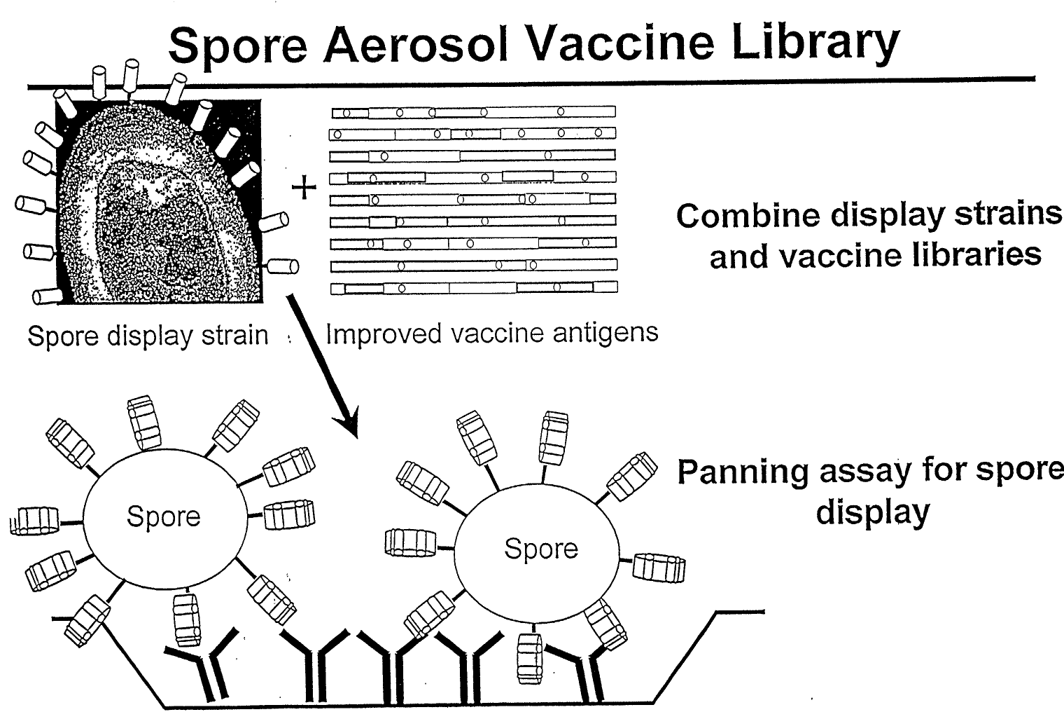

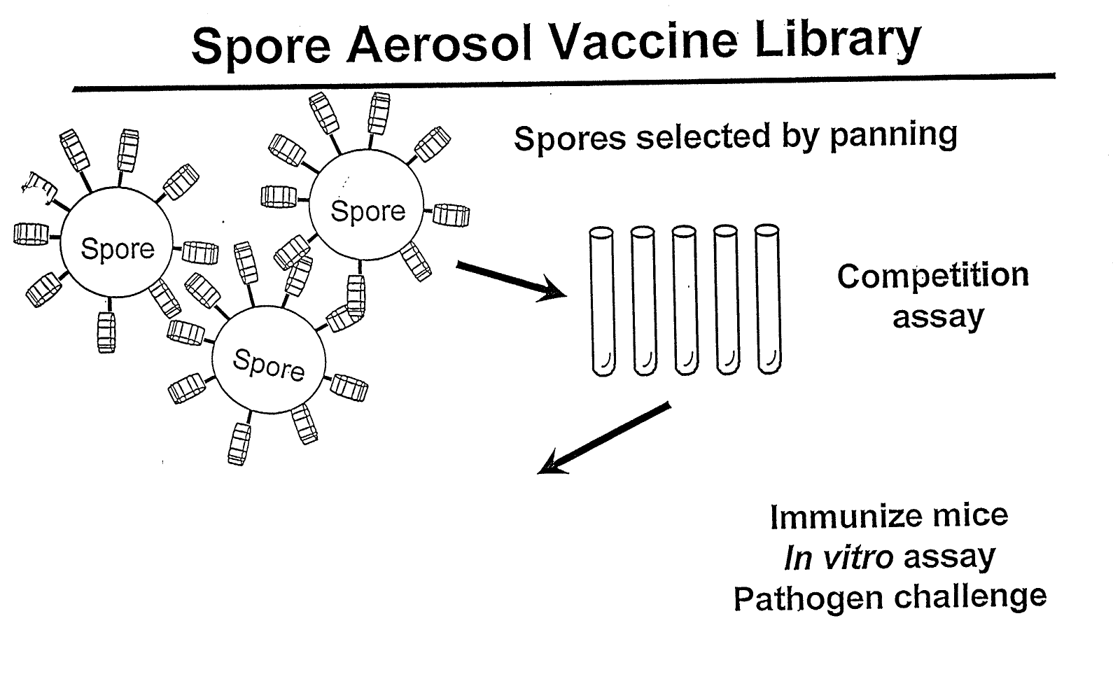

Another method for identifying proteins, polypeptides, and peptides having the desired antigenic properties involves multi-tiered screening. In this method, a strain, which may contain a mutagenized library, is put through a multistep selection process to collect a pool of mutant strains showing higher antigenicity than the appropriate control strain. The multistep selection process of multi-tiered screening (illustrated in FIG. 1) comprises a first step of spore selection (e.g., panning, Fluorescent Activated Cell Sorting (FACS) sorting, and the like), a second step of a competition assay to select the most appropriate antigen, and a third step of an in vitro assay or ex vivo or in vivo assay in an appropriate subject, for example, a mammal, including, e.g., a non-human primate, mouse or rabbit. The bacterial spores remaining after the third step represent a pool containing the best antigen candidate sequences. Such antigen candidate sequences can then be tested for utility as a vaccine using any appropriate in vitro, ex vivo, or in vivo assay. For example, the in vivo mouse assay may be used to test efficacy of a vaccine by challenging the vaccinated mouse with the pathogen against which the vaccine was intended to protect. An in vivo assay may be a final step in any of a variety of multistep selection and screening processes, such as the one illustrated in FIG. 3. In vitro assays for predicting efficacy of vaccines are also available in the art. Such in vitro assays include, e.g., a panning assay, FACS sorting, and the like, to identify bacteria expressing polypeptides and other molecules of interest, as diagrammed in FIG. 2. One of skill in the art will appreciate that the use of particular screening or selection methods and the number of steps involved can include any or all of the methods and steps mentioned or referred to herein, and will also appreciate that the order of these steps may vary according to the result sought, the properties of the polypeptides or other molecules of interest, the bacterial strain(s) used in the process, and other factors.

Appropriate antigens, polypeptides, peptides, nucleic acids, immunomodulatory molecules, or other molecules of interest can then be used in a spore system to elicit or alter an immune response in a subject. In one embodiment, for example, the antigen or polypeptide of interest is displayed on the surface of the spore in a spore display system so that the antigen or polypeptide comes into contact with the cells of the host animal airways or vasculature and elicits an immune response. For example, the polypeptide or antigen can be delivered into the host animal airways using a spore delivery system. Using a spore delivery system to deliver polypeptides and vaccine antigens to a subject provides a formulation that is resistant to degradation by heat, light, and shear stresses. Where antigens, polypeptides, nucleic acids, and other molecules of interest were inactivated by desiccation of the spore delivery system, the spores could be stored in sterile water or buffer. The spore system thus lends itself to delivery to a subject as an aqueous or nonaqueous solution or suspension or a dry powder form. Spore systems provide a means for the storage of immunomodulatory molecules, including genetic vaccines, protein vaccines, and adjuvants at ambient temperature and also provide easy administration and/or vaccine immunization. A spore system-based therapeutic or prophylactic agent or vaccine is easy and inexpensive to produce and has reduced storage costs; thus, the present invention has clear administration and cost advantages.

Methods for administering spore systems, spore display systems, and spore encapsulate systems of the present invention include those known to those having ordinary skill in the art. Suitable routes of administration or “delivery systems” include parenteral delivery and enteral delivery, such as, for example, oral, transdermal, transmucosal, intravenous, subcutaneous, intramuscular, intradermal, intraperitoneal, intracapsular, intraspinal, intrastemal, intrapulmonary, intranasal, vaginal, rectal, intraocular, and intrathecal, buccal (e.g., sublingual), respiratory, topical, ingestion, and local delivery, such as by aerosol or transdermally, and the like. Methods for administering proteins, polypeptides, peptides, nucleic acids, and other molecules of interest to mucosal tissue via pulmonary inhalation, nasal, oral, vaginal, and/or rectal delivery are provided. The methods comprise preparing and administering to a subject a composition comprising a spore system of the present invention. Such composition may include a carrier or excipient. In one embodiment of the invention, a polypeptide, protein, peptide, nucleic acid, or other molecule of interest is displayed on the surface of the spore. In another embodiment, the polypeptide, protein, or peptide of interest is expressed by the vegetative cells resulting from the germination and/or vegetative reproduction of a spore. In yet another embodiment, the spore displays a polypeptide, protein, or peptide with DNA binding capabilities that is bound to a DNA molecule encoding an antigen or immunomodulatory molecule or that is an antigen or immunomodulatory molecule.

Methods of delivery of a spore system, including a vaccine or composition comprising a spore system or treatment of a subject or a population of subjects with any one or combination of such systems are provided. Spores are small enough to be readily airborne; thus, spore-based vaccines or spore system compositions can be delivered by air or by aerosolization, which provides a simple, rapid, and inexpensive means of inoculating or treating subject populations. In one embodiment of the invention, a human population could be inoculated against a threat of biological warfare by a vaccine or treatment agent comprising one or more of the systems of the invention distributed by means of the air, water, or food supply. Such distribution could be accomplished by introduction of the vaccine or treatment agent into the air, water, or food supply by any of various means, including delivery by distribution by airplanes, helicopters, ships, or other means of air or water transportation. For example, such a vaccine could be sprayed over an area by a plane adapted for crop dusting.

These methods of inoculation and/or immunization can also be utilized for herd animals in a field, such as cattle grazing over an extended area, or for fish in their native aquatic habitats. Subject animals also include wild animals. For example, subjects include American buffalo (bison), which often carry the disease brucellosis, which can infect humans and causes spontaneous abortions in cattle. In another embodiment, rabies vaccinations or therapeutic or prophylactic agents comprising spore systems of the invention are administered to a variety of wild animal populations in a particular area by distributing spores from an overflying plane. Thus, the present invention provides a relatively inexpensive means for vaccinating or treating wild populations against a variety of illnesses and diseases. Diseases and illnesses that are potential targets of this vaccination approach include all those described above, including, e.g., those caused by cholera (e.g., enterotoxins from V. cholerae), Japanese encephalitis, tick-borne encephalitis, Venezuelan Equine encephalitis, enterotoxins produced by Staphylococcus and Streptococcus species, and enterotoxigenic strains of E. coli (e.g., heat-labile toxin from E. coli), and salmonella toxin, shigella toxin and campylobacter toxin, dengue fever, and hantavirus.

Distribution of the vaccine or other prophylactic or therapeutic agent comprising a spore system of the invention to fish in the aquaculture or aquarium trades can be accomplished by injection or immersion techniques. Immersion, or dipping, is an inoculation or vaccination method well known to one of skill in the art (see e.g., Vinitnantharat et al. (1999) Adv. Vet. Med. 41:539-550). A dip treatment involves dipping whole fish in a dilution of the inoculant or vaccine whereupon the inoculant or vaccine is absorbed by the gills. Intraperitoneal injection is another vaccination method well known to one of skill in the art. Injection involves anesthetizing and injecting the fish intraperitoneally (Vinitnantharat et al. (1999) Adv. Vet. Med. 41:539-550). Diseases of cultivated fish that may be treated using a spore system of the invention include, but are not limited to, infectious pancreatic necrosis (IPN), infectious hematopoietic necrosis (IHH), Vibriosis (Vibrio anguillarum), cold-water vibriosis (Vibrio salmonicida), Vibrio ordalii, winter ulcer (Vibrio viscosus), Vibrio wodanis, yersiniosis (Yersinia ruckeri) or Enteric Red Mouth, Bacterial Kidney Disease, Furunculosis (Aeromonas salmonicida subsp. salmonicida), Saddleback, Gafkemia, Dollfustrema vaneyi, Cryptobia bullocki, Cryptobia salmositica, Listeria monocytogenes, Photobacterium damsela subsp. piscicida, Microcotyl sebastis. Fish species of interest include, but are not limited to, salmonids, including Rainbow Trout (Onchorhycus mykiss), salmon (Salmo salar), Coho salmon (Oncorhynchus kisutch), rockfish (Sebastis schlegeli), catfish (Ictalurus punctatus), yellowtail, Pseudobagrus fulvidraco, Gilt-head Sea Bream, Red Drum, European Sea Bass fish, striped bass, white bass, yellow perch, whitefish, sturgeon, largemouth bass, Northern pike, walleye, black crappie, fathead minnows, and Golden Shiner minnows. Invertebrates of interest include, but are not limited to, oysters, shrimp, crab, and lobsters.

Methods for enhancing bioavailability of the polypeptide or other molecule of interest are encompassed by the invention. In one embodiment, the method comprises preparing an aerosol or other suitable preparation of the highly absorbable compositions disclosed herein and administering an aerosolized preparation to the subject via pulmonary inhalation, for example, by use of an inhaler device. In such methods, the preparation may contain adjuvants and/or other ingredients to enhance the suitability of the preparation for pulmonary administration.

In one embodiment of the invention, the spore component of the spore system functions as an adjuvant for oral, parenteral (e.g., subcutaneous, intramuscular, intradermal, or intravenous), respiratory, nasal, pulmonary topical, rectal, vaginal, mucosal, intrathecal, buccal (e.g., sublingual), or local delivery, such as by aerosol, ingestion, or transdermally. By mucosal delivery is intended delivery across any of the mucous membranes of the organism.

Delivery by pulmonary inhalation, nasal delivery, gill delivery, or respiratory delivery provides a promising route for absorption of polypeptides and other molecules of interest having poor oral bioavailability due to inefficient transport across the gastrointestinal epithelium or high levels of first-pass hepatic clearance. By “nasal delivery” is intended that the polypeptide is administered to the subject through the nose. By “pulmonary inhalation” is intended that the polypeptide or other substance of interest is administered to the subject through the airways in the nose or mouth so as to result in delivery of the polypeptide or other substance to the lung tissues and into the interior of the lung. Both nasal delivery and pulmonary inhalation can result in delivery of the polypeptide or other substance to the lung tissues and into the interior of the lung, also referred to herein as “pulmonary delivery.” By “respiratory delivery” is intended that the polypeptide or other substance is administered to the subject through the respiratory system of the subject so as to result in delivery of the polypeptide or other substance to the organs and tissues of the respiratory system of the subject organism. The organs and tissues of the respiratory system of a subject organism include, but are not limited to, the lungs, nose, or gills. Potential advantages of these delivery routes for polypeptides and other molecules of interest include a greater extent of absorption due to an absorptive surface area of approximately 140 m² and high volume of blood flowing through the lungs (5000 ml/min in the human lung) (Hollinger (1985), pp. 1-20, in Respiratory Pharmacology and Toxicology (Saunders, Pa.)). Further potential benefits of administration via pulmonary inhalation include lack of some forms of peptidase and/or protease activity when compared with the gastrointestinal tract and lack of first-pass hepatic metabolism of absorbed compounds. Interest in this delivery route has increased in recent years since a number of potential peptide-, polypeptide-, or protein-containing pharmaceuticals or drugs are absorbed more efficiently from the lung than from the gastrointestinal tract (Patton and Platz (1992) Adv. Drug Del. Rev. 8:179-196; Niven (1993) Pharm. Technol. 17:72-82). In fish, respiratory delivery of vaccines is the primary mode of vaccination due to the technical difficulties associated with injection of each fish and the destruction of most vaccines in the digestive tract of the fish.

Delivery of peptide-, protein-, or polypeptide-containing pharmaceutical formulations via pulmonary inhalation is known, although only a few examples have been quantitatively substantiated. See, for example, Hubbard et al. (1989) Ann. Internal Med. 3(3): 206-212 (plasma α-1-antitrypsin); Smith et al. (1989) J. Clin. Invest. 84: 1145-1154 (α-1-proteinase inhibitor). Experiments with test animals have shown that recombinant human growth hormone, when delivered by aerosol, is rapidly absorbed from the lung and produces faster growth comparable to that seen with subcutaneous injection (Oswein et al. (1990), “Aerosolization of Proteins” in Proceedings of Symposium on Respiratory Drug Delivery II (Keystone, Colorado, March, 1990)). Recombinant versions of the cytokines gamma interferon (IFN-γ) and tumor necrosis factor alpha (TNF-α) have also been observed in the bloodstream after aerosol administration to the lung (Debs et al. (1988) J. Immunol. 140: 3482-3488). The feasibility of pulmonary delivery of granulocyte-colony stimulating factor (G-CSF) and erythropoietin (EPO) to mammals has also been demonstrated (U.S. Pat. Nos. 5,284,656 and 5,354,934, respectively). See also U.S. Pat. No. 5,997,848, where systemic delivery of insulin to a mammalian host is accomplished via inhalation of a dry powder aerosol containing insulin.

Successful respiratory delivery of peptides, polypeptides, or proteins is dependent upon a number of factors but delivery can be readily optimized by varying such factors in routine experimentation by one of skill in the art. The extent of absorption within the respiratory tissues varies with size and structure of the polypeptide, peptide, or protein and the delivery device used. Spore systems, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like. Delivery devices include nebulizers, metered-dose inhalers, powder inhalers, and dipping bags. Preparation of compositions, including those comprising spore systems, as an aqueous liquid aerosol, a nonaqueous suspension aerosol, or dry powder aerosol for pulmonary administration using these respective delivery devices can influence polypeptide stability, and hence bioavailability as well as biological activity following delivery. See Wall (1995) Drug Delivery 2:1-20; Krishnamurthy (March 1999) BioPharm., pp. 34-38). The enhanced stability of the spore systems of the present invention is therefore of value in administration by respiratory delivery.

Respiratory delivery provides an attractive noninvasive alternative to intravenous or subcutaneous administration. Pulmonary inhalation of polypeptides, proteins, and peptides has been demonstrated using a nebulizer to deliver an aqueous liquid formulation containing IL-2 (U.S. Pat. Nos. 5,399,341 and 5,780,012). However, pulmonary administration of polypeptides as an aerosol using the nebulizer system has been shown to denature some polypeptides (see Ip et al. (1995) J. Pharm. Sci. 84:1210-12-14 (interferon); Niven et al (1994) Int. J. Pharm. 109: 17-26 (recombinant granulocyte-colony-stimulating factor); and Niven et al. (1995) Pharm. Res. 12:53-59). During the nebulization process, the polypeptide is exposed to shearing stresses that may aggravate loss of biological activity, which is minimized or eliminated by the spore systems of the present invention. Thus, the spore systems of the invention may be administered by parenteral means including, but not limited to, subcutaneous, intraperitoneal, intramuscular, and intravenous introduction, e.g., as via injection, gene gun, vaccine gun, or impressing through the skin. Spore systems of the invention can be administered in ex vivo or in vivo therapy parenterally. It will be appreciated that the delivery of such systems to subjects is routine, e.g., delivery of spore systems or compositions thereof to the blood via intravenous, intramuscular, or intraperitoneal administration or other common route. Formulations suitable for parenteral administration, such as, for example, by intraarticular (in the joints), intravenous, intramuscular, intradermal, subdermal, intraperitoneal, and subcutaneous routes, include aqueous and non-aqueous, isotonic sterile injection solutions, which can contain antioxidants, buffers, bacteriostats, and/or solutes that render the formulation isotonic with the blood of the intended recipient, and aqueous and non-aqueous sterile suspensions that can include suspending agents, solubilizers, thickening agents, stabilizers, and preservatives. In the practice of the invention, spore systems and compositions comprising spore systems can be administered, for example, by intravenous infusion, orally, topically, intraperitoneally, intravesically or intrathecally.

Formulations suitable for oral administration can comprise, but are not limited to: (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. It is recognized that the genetic vaccines, when administered orally, may need to be protected from digestion. This is typically accomplished either by complexing the vaccine vector with a composition to render it resistant to acidic and enzymatic hydrolysis or by packaging the vector in an appropriately resistant carrier such as a liposome. Means of protecting vectors from digestion are well known in the art. The pharmaceutical compositions can be encapsulated, e.g., in liposomes, or in a formulation that provides for slow release of the active ingredient.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

The formulations of packaged spore systems and nucleic acids, peptides, proteins, or polypeptides of the invention can be presented in unit-dose or multi-dose sealed containers, such as ampules and vials. Solutions and suspensions for parenteral and other forms administration can be prepared from sterile powders, granules, and tablets of the kind previously described.

The dose administered to a subject, in the context of the present invention, is typically sufficient to effect a beneficial effect, such as an altered immune response or other therapeutic and/or prophylactic response in the subject over time, or to, e.g., inhibit infection by a pathogen, depending on the application. The dose will be determined by the efficacy of the particular nucleic acid, polypeptide, protein, peptide, vector, expression cassette, formulation, or other component of the spore system or composition thereof, and/or the activity of any such component to be administered or employed, and the condition of the patient, as well as the body weight, surface area, or vascular surface area, of the subject to be treated. The size of the dose also will be determined by the existence, nature, and extent of any adverse side-effects that accompany the administration of any such particular polypeptide, protein, peptide, nucleic acid, vector, expression cassette, formulation, or other component of the spore system or composition thereof, or the like in a particular subject.

Dosages to be used for therapeutic or prophylactic treatment of a particular disease or disorder can be determined by one of skill by comparison to those dosages used for existing therapeutic or prophylactic treatment protocols for the same disease or disorder. In determining the effective amount of the polypeptide, protein, peptide, nucleic acid, vector, expression cassette, formulation, or other component of the spore system or composition thereof to be administered to a subject for the treatment or prophylaxis of the medical condition or disease state (e.g., allergies, cancers, or viral diseases), a physician or veterinarian evaluates the subject for, e.g., circulating plasma levels, toxicities to the polypeptide, protein, peptide, nucleic acid, vector, expression cassette, formulation, or other component of the spore system or spore itself, progression of the disease or condition, and the production of antibodies to the polypeptide, protein, peptide, formulation, etc. or other component of the spore system or spore itself, and, depending on the subject, other factors that would be known to one of skill in the art.

In one aspect, for example, in determining the effective amount of the spore system to be administered in the treatment or prophylaxis of an infection, disease, or other condition, wherein the spore system comprises an expression vector comprising a nucleic acid that encode a polypeptide of interest, the physician or veterinarian evaluates vector toxicities, progression of the infection, disease, or other condition, and the production of anti-vector or anti-polypeptide antibodies, if any. In one aspect, the dose equivalent of a naked nucleic acid from a vector or the dose equivalent of a polypeptide of interest for a typical 70 kilogram subject can range from about 10 ng to about 10 g, about 100 ng to about 5 g, about 500 ng to about 1 g, about 100 mg to about 500 mg, about 100 fig to about 100 mg, about 50 μg to about 50 mg, about 10 μg to about 10 mg, 30 μg to about 1 mg, or about 30 μg to about 300 μg.

Dose can also be determined as the number of moles (or grams) of polypeptide of interest displayed on the spore (e.g., antigen) per number of spores administered. ELISAs can be used to determine the anti-spore response, spore concentration, and response of mice to specific antigens displayed on the spore (see, e.g., Example 3). For example, using the spore concentration and antigen concentration, the number of moles of antigen (e.g., micrograms) per number of spores can be determined. The number of spores displaying a molecule of interest (e.g., antigen) sufficient to deliver an effective amount of the molecule of interest can be readily determined (see, e.g., Ex. 3). In some instances, the dose (e.g., micrograms) of a nucleic acid, peptide, protein, or polypeptide commonly used for a particular therapeutic or prophylactic treatment or immunization (e.g., in a vaccine) may be decreased by using such nucleic acid, protein, peptide, or polypeptide in combination with a population of spores (e.g., about 5×10⁵to about 5×10⁹ spores), such as, e.g., in a spore system of the present invention. For humans, for example, the effective dose of recombinant HepBsAg protein vaccine ranges from about 2.5 micrograms (for children) to about 20 to about 40 micrograms (for adult), depending on the formulation (Drug Information Handbook, L. Lance et al., Lexi-Comp's Clinical Reference Library (5th ed. 1998-99)). For delivery of the HepBsAg protein in conjunction with a spore system of the invention, for example, an equal or lower amount of hepBsAg may be sufficient to induce a protective response against the virus, particularly given the adjuvant effect of the spore itself. The number of spores required to deliver such dose can be readily determined by the assays described herein.

Doses of vectors used to deliver nucleic acids are calculated to yield an equivalent amount of therapeutic or prophylactic nucleic acids. Administration can be accomplished via single or divided doses. If desired, additional doses can be administered following initial administration at selected intervals of time. For example, for vaccine or immunization applications, additional doses of the spore system or compositions thereof, can be administered to “boost” effectively the initial therapeutic or prophylactic dose (e.g., vaccine dose). Such additional doses may be at the same level as the initial dose or an augmented or lower dose, depending upon the application. One of skill in the art can readily adjust any such dose depending on the application and severity of the disease or condition to be treated. Additional doses can be in the same format as the initially administered therapeutic or prophylactic dose or in a different format as described herein. For example, if an initial immunization comprises a vaccine comprising a recombinant spore system comprising a DNA expression vector encoding a polypeptide or antigen or interest, the subsequent “boosting” immunization may comprise, if desired, a recombinant spore system without the expression vector, but comprising the polypeptide, antigen, or similar antigenic peptide displayed on or coupled to the spore surface (e.g., via avidin linkage or via binding to the positively charged amino acids on the surface of the spore).

Thus, the present invention provides advantages over previously known methods of vaccination. First, compositions of the present invention can easily be prepared in large amounts by growing and sporulating the strain of the spore system in large fermentors. This aspect of the invention provides an important advantage in extending the capacity to quickly scale up production. For example, a spore system providing a vaccine could be rapidly produced to respond to a widespread outbreak of disease. Another aspect of the present invention is its provision of an exceptional means of storage of vaccine stocks; a vaccine may be stored in a spore system at ambient temperature for an indefinite length of time, possibly up to hundreds of years.

A spore system of the invention, or a composition comprising such spore system, can be used in a variety of medicinal, therapeutic, prophylactic, and pharmaceutical methods and applications described herein. The invention provides for the use of any such spore system, or any composition or combination thereof, as a medicament, vaccine, or therapeutic, prophylactic, or immunomodulatory agent, for the treatment or prevention of any of the diseases or conditions described herein. The invention further provides for the use of any such spore system or any composition or combination thereof, for the manufacture of medicament, vaccine, or therapeutic, prophylactic, or immunomodulatory agent, for any application or method relating to the treatment or prevention of any of the diseases or conditions described herein.

Antibody Applications

Methods of identifying antibodies and antibody fragments that selectively bind the polypeptides and other molecules of interest are provided. For example, a polypeptide or polynucleotide of interest can be used in a spore system as an antigen or immunogen to stimulate the production of and/or to identify antibodies that bind the polypeptide of interest. In this way, the present invention can be used in conjunction with standard techniques for polyclonal and monoclonal antibody preparation.

Accordingly, another aspect of the invention pertains to an improved method for identification and isolation of polyclonal and monoclonal antibodies that bind the polypeptide or polynucleotide of interest. Polyclonal antibodies to the polypeptide of interest can be prepared, e.g., by aerosol administration of a spore system of the present invention to a suitable subject. Such subjects include birds and reptiles as well as mammals (e.g., rabbit, goat, mouse, or other mammal). The antibody titer in the serum of an immunized subject can be monitored over time by standard techniques, such as with an enzyme linked immunosorbent assay (ELISA) using immobilized polypeptide. At an appropriate time after immunization, e.g., when the appropriate antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4: 72), the EBV-hybridoma technique (Cole et al. (1985), pp. 77-96 in Monoclonal Antibodies and Cancer Therapy, Reisfeld and Sell, eds. (Alan R. Liss, Inc., New York, N.Y.)) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:550-52; Kenneth (1980) in Monoclonal Antibodies: A New Dimension In Biological Analyses (Plenum Publishing Corp., NY); and Lemer (1981), Yale J. Biol. Med., 54:387-402).

Additionally, recombinant antibodies, such as chimeric and humanized monoclonal antibodies, which can be made using standard recombinant DNA techniques, are within the scope of the invention. Such chimeric and humanized monoclonal antibodies can be produced by recombinant DNA techniques known in the art, for example using methods described in PCT Publication Nos. WO 86/101533 and WO 87/02671; European Patent Application Nos. 184,187, 171,496, 125,023, and 173,494; U.S. Pat. Nos. 4,816,567 and 5,225,539; European Patent Application 125,023; Better et al. (1988) Science 240: 1041-1043; Liu et al. (1987) Proc. Natl. Acad. Sci. USA 84: 3439-3443; Liu et al. (1987) J. Immunol. 139: 3521-3526; Sun et al. (1987) Proc. Natl. Acad. Sci. USA 84: 214-218; Nishimura et al. (1987) Canc. Res. 47: 999-1005; Wood et al. (1985) Nature 314: 446-449; Shaw et al. (1988) J. Natl. Cancer Inst. 80: 1553-1559); Morrison (1985) Science 229: 1202-1207; Oi et al. (1986) Bio/Techniques 4: 214; Jones et al. (1986) Nature 321: 552-525; Verhoeyan et al. (1988) Science 239: 1534; and Beidler et al. (1988) J. Immunol. 141: 4053-4060.

Yet another aspect of the invention pertains to antibodies identified by use of such methods. Such antibodies are useful in detecting the polypeptides of interest as well as in regulating the T-cell immune response and cellular activity, particularly growth and proliferation. In another embodiment of the present invention, the polypeptide of interest used in the spore system is an antibody molecule. In some embodiments, the polypeptide of interest is a single-chain antibody. See, for example, Verma et al., J. Immunol. Methods 216(1-2): 165-181 (1998). Spore systems displaying or incorporating antibody molecules can be used in immunoassays and the like, or for therapeutic or prophylactic treatment. Such antibodies, or antibodies prepared against such antibodies, may be useful in the treatment or prevention of, e.g., autoimmune diseases.

Completely human antibodies are particularly desirable for therapeutic treatment of human patients. Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994), Bio/Technology 12:899-903.

An anti-polypeptide-of-interest antibody (e.g., monoclonal antibody) can be used to isolate proteins sharing characteristics of the polypeptide of interest by standard techniques, such as affinity chromatography or immunoprecipitation. An anti-polypeptide-of-interest antibody can facilitate the purification of natural polypeptide-of-interest from cells and of recombinantly produced polypeptide of interest that is expressed in host cells. Moreover, an antibody which binds to the polypeptide of interest can be used to detect polypeptides similar to the polypeptide of interest (e.g., in a cellular lysate or cell supernatant) in order to evaluate the abundance and pattern of expression of similar proteins. Such antibodies can also be used diagnostically to monitor detectable compounds, including protein levels in tissue, as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Further, an antibody (or fragment thereof) may be conjugated to a therapeutic or prophylactic moiety such as a cytotoxin, a therapeutic or prophylactic agent, or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic or prophylactic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic or prophylactic agents. For example, the drug moiety may be a protein, polypeptide, or peptide possessing a desired biological activity. Such polypeptides or proteins may include, for example, a toxin such as abrin, ricin A, pseudomonas exotoxin, or diphtheria toxin; a polypeptide or protein, such as tumor necrosis factor, alpha-interferon, beta-interferon, nerve growth factor, platelet derived growth factor, tissue plasminogen activator; or biological response modifiers, such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moieties to antibodies are well known, see, e.g., Arnon et al. (1985), “Monoclonal Antibodies For Immunotargeting Of Drugs In Cancer Therapy”, pp. 243-56 in Monoclonal Antibodies And Cancer Therapy, Reisfeld et al. (eds.) (Alan R. Liss, Inc.); Hellstrom et al. (1987), “Antibodies For Drug Delivery”, pp. 623-53 in Controlled Drug Delivery (2nd edition), Robinson et al. (eds.) (Marcel Dekker, Inc.); Thorpe (1985), “Antibody Carriers Of Cytotoxic Agents In Cancer Therapy: A Review”, pp 475-506 in Monoclonal Antibodies '84: Biological And Clinical Applications, Pinchera et al. (eds.); “Analysis, Results, And Future Prospective Of The Therapeutic Use Of Radiolabeled Antibody In Cancer Therapy”, pp. 303-316 in Monoclonal Antibodies For Cancer Detection And Therapy, Baldwin et al. (eds.) (Academic Press 1985), and Thorpe et al. (1982), “The Preparation And Cytotoxic Properties Of Antibody-Toxin Conjugates”, Immunol. Rev., 62:119-58. Alternatively, an antibody can be conjugated to a second antibody to form an antibody heteroconjugate as described by Segal in U.S. Pat. No. 4,676,980.

Compositions

The invention also includes compositions of the spore systems and components thereof. Compositions may further comprise a carrier. As used herein the language “carrier” is intended to include any and all solvents, dispersion media, coatings, antibacterial and antifungal agents, isotonic and absorption delaying agents, excipients, and the like, compatible with pharmaceutical, agricultural, or aquacultural administration. Carriers may be liquid or fluid in form. A carrier may be a pharmaceutically acceptable carrier for use in any composition described herein or a pharmaceutical or nutraceutical composition (see, e.g., below). The use of such media and agents for pharmaceutically active substances is well known in the art. Except insofar as any conventional medium or agent is incompatible with the spore, spore system, or active component thereof (e.g., spore system comprising an immune-system-enhancing DNA vector or polypeptide), use of such medium or agent in the compositions is contemplated. Supplementary active substances or compounds (including, but not limited to, e.g., nucleic acids, polypeptides, peptides, cytokines, co-stimulatory molecules) can also be incorporated into the compositions.

Pharmaceutical and nutraceutical compositions comprising spore systems are encompassed by the present invention. The term “pharmaceutical composition” typically refers to a composition suitable for pharmaceutical use in a subject, including an animal or human. A pharmaceutical composition generally comprises an active agent (e.g., spore system alone or spore system comprising a nucleic acid, polypeptide or other molecule of interest as described herein) and pharmaceutically acceptable carrier or excipient. In some aspects, the pharmaceutical composition comprises an effective amount of an active agent and a carrier, such as a pharmaceutically acceptable carrier. The term “effective amount” typically means a dosage or amount sufficient to produce a desired result. The desired result may comprise an objective or subjective improvement in the recipient of the dosage or amount. Pharmaceutical formulations, including media, agents, and carriers, are well known in the art and can be used with pharmaceutical compositions comprising spore systems of the invention. Conventional pharmaceutical media or agents in the pharmaceutical compositions is contemplated except where such media or agents are incompatible with the spore, spore system, or component thereof.

In addition to components described above, such pharmaceutical compositions may comprise appropriate stabilizing agents, bulking agents, or both to minimize problems associated with loss of protein stability and biological activity during lyophilization, spray-drying, and aerosolizing processes included in the pulmonary administration methods of the invention. Such compositions may further include stabilizing or bulking agents, such as those used for conventional pharmaceutical compositions not containing spore systems or spore delivery systems.

Such pharmaceutical compositions are suitable for administration in any suitable manner, including parenteral (e.g., subcutaneous, intramuscular, intradermal, or intravenous), topical, oral, rectal, vaginal, intrathecal, buccal (e.g., sublingual), inhalation, pulmonary, transdermal, or by aerosol administration or delivery, for immunotherapeutic or other prophylactic and/or therapeutic treatment are included. Suitable methods of administering such packaged nucleic acids, polypeptides, peptides, proteins, carbohydrates and other molecules of interest are available and well known to those of skill in the art, and although more than one route can be used to administer a particular composition, a particular route can often provide a more immediate and more effective reaction than another route.

Industrial Applications

In yet another embodiment, the present invention provides a method and compositions for controlled delivery of enzymes, for example, to particular locations at particular times. Such methods and compositions may find particular use in industrial processes such as bioremediation, biochemical processes, pulp and paper processing, and the like.

Spore display systems of the present invention can be used as the source of a wide variety of enzymes and non-enzyme polypeptides having industrial, biomedical, and biotechnological uses. The polypeptides to be displayed, incorporated, or expressed may originate in any species and can be either monomeric or multimeric. Such polypeptides may be enzymes that are useful in detergent formulations, such as lipases, proteases, amylases, and the like. Alternatively, such polypeptides may be enzymes that are useful for a variety of industrial or biosynthetic processes. Such enzymes include, but are not limited to, glucose oxidase, galactosidase, glucosidase, nitrilase, alkene monooxygenase, hydroxylase, aldehyde reductase, alcohol dehydrogenase, D-hydantoinase, D-carbamoylase, L-hydantoinase, L-decarbamoylase, beta-tyrosinase, dioxygenase, serine hydroxy-methyltransferase, carbonyl reductase, nitrile hydratase, o-phthalyl amidase, halohydrin hydrogen-halide lyase, maltooligosyl trehalose synthase, maltooligosyl trehalose trehalohydrolase, lactonase, oxygenase, adenosylmethionine synthetase, cephalosporinase, fucosidase, adenosylhomocysteine hydrolase, peroxidase, nucleoside phosphorylase, hemicellulase, cyclodextrin glycosyltransferase, oxidase, endoglucanase, polygalacturonase, amylase, glutamyl endopeptidase, xylanase, laccase, phenol oxidase, cellulase, lactate oxidase, neuraminidase, ribonuclease, lipase, esterase, aldolase, oxynitrilase, lyase, protease, acylase, glucose isomerase, amidase, phosphotransferase, kinase, dephosphorylase, phosphatase, epoxide hydrolase, P450 monooxygenase, toluene monooxygenase, methane monooxygenase, and other enzymes. Such enzymes are known in the art; see, for example, Ogawa and Shimizu (1999), Trends in Biotechnology 17:13-20; Singh et al. (2000), J. Appl. Microbiol. 88(6):975-982, and references cited therein. Enzymes that may be used in spore systems of the present invention include proteins that interfere with mammalian cell viability or protein assembly in mammalian cell expression systems, such as retinoblastoma protein and leptin.

By “enzymatic activity” is intended any modification of a molecule including, but not limited to, ligation or formation of chemical bonds, oxidation, reduction, the addition or deletion of a chemical moiety, or any other change that affects the activity or structure of a molecule. By “substrate” is intended a starting compound, molecule, or substance. By “alters” is intended a modification or change of structure or activity including, but not limited to, ligation or formation of chemical bonds, oxidation, reduction, the addition or deletion of a chemical moiety.

Polypeptides of interest can be presented on the surface of the spore so as to interact directly with other enzymes or substances in the environment surrounding the spore system. Advantages of these aspects of the invention are similar to the advantages provided by the present invention for vaccinations: rapid scale-up is possible, storage is relatively insensitive to temperature fluctuations and the passage of time, and the spore delivery system provides a means for controlling delivery of the polypeptide of interest. Additionally, the spore encapsulate system of the present invention has the advantage that it provides a means for delivering encapsulated polypeptides to a process in a controlled, time-defined or location-defined manner which is provided with relative ease and at reduced expense. An added benefit of the present invention is that spores tend to sink in solution, thus eliminating the need to centrifuge a solution to remove said spores. The spore delivery system of the present invention may be stored at room temperature, either in dried form or in an aqueous buffer with stabilizers to preserve or enhance the activity of the polypeptides of interest.

Spore delivery systems displaying or incorporating different polypeptides could be used in sequence to perform a multistep synthetic or degradative process or chemical synthesis. For example, polypeptide of interest A catalyzes the conversion of the initial substrate to an intermediate substrate which is acted upon by polypeptide of interest B to yield a second intermediate substrate which is acted upon by polypeptide of interest C to yield a final product. The proximity of the enzymes to the intermediate substrates produced by the preceding enzymes in the pathway increases the reaction rate of the pathway thus decreasing the total amount of time required for the pathway and increasing the concentration of the final product. In an embodiment of the invention, the spore system displays polypeptides necessary for more than one step in a biochemical pathway.

The spore systems of the invention may be used in the production or modification of any biosynthetic compound including, but not limited to, peptides, polypeptides, carbohydrates, fatty acids, hormones, steroids, lipids, lipoproteins, glycoproteins, sugars, organic acids, esters, ketones, nucleic acids, cations, anions, enzymes, protease inhibitors, growth factors, and alcohols. For example, industrial production of lactic acid is performed in large fennentors of bacteria or fungi. As the organisms produce lactic acid, the increasing lactic acid concentration lowers the pH of the growth media below the optimum range for growth and vitality of the organisms. The presence of lactic acid in the media inhibits high yield production of lactic acid. The spore system of the invention may be used to increase the lactic acid yield. When the spore system of the invention displays an enzyme that polymerizes lactic acid, the lactic acid is polymerized into dimers or higher order multimers thereby reducing the concentration of lactic acid in the media. The spore system facilitates maintenance of low lactic acid concentrations which allows the media to be maintained in the optimal pH range for the organisms. The organisms continue to produce lactic acid thus increasing the lactic acid yield. The multimeric lactic acid molecules can be depolymerized after the bioreactor is harvested.

A further benefit of the present invention is provided by the resilience to environmental insult exhibited by spores. Thus, spore systems of the present invention have superior chemical resistance and could be used in applications where chemical resistance was beneficial. For example, chemical resistance of a method of treatment might be beneficial in treatment of contaminated soils in a bioremediation process. Spore systems have enhanced survivability in such situations relative to non-spore systems, thus permitting bioremediation at a reduced cost. In such situations, the spore is modified to display, incorporate, or express enzymes having activities useful in bioremediation, which may include enzymes such as chromate reductase (see Park et al. (2000), Appl. Environ. Microbiol. 66(5): 1788-95), peroxidase or laccase (see Park et al. (2000), Arch. Environ. Contam. Toxicol. 38(4): 405-410), molybdenum hydroxylase or ring-opening 2,4-dioxygenase (Fetzner (2000), Naturwissenschaften 87(2): 59-69), catechol 2,3-dioxygenase (Mesarch et al. (2000), Appl. Environ. Microbiol. 66(2): 678-83), haloalkane dehalogenases (Newman et al. (1999), Biochemistry 38(49): 16105-14), pentaerythritol tetranitrate reductase (French et al. (1999), Nat. Biotechnol. 17(5): 491-94), toluene dioxygenase (Lange et al. (1998), Nat. Biotechnol. 16(10): 929-33), phenol monooxygenase (Peters et al. (1997), Appl. Environ. Microbiol. 63(12): 4899-4906). See generally for a discussion of engineering for bioremediation, Chen et al. (1999), Curr. Opin. Biotechnol. 10(2): 137-41. In bioremediation processes or applications, it may be useful to combine various enzymatic activities with each other as well as with other chemicals to improve the result of the process. These various enzymatic activities may be displayed on the same spore. See, for example, Park et al. (2000), Arch. Environ. Contam. Toxicol. 38(4): 405-410.

In an embodiment, the spore system of the present invention can be used to provide anti-microbial agents for industrial, medical, commercial or residential use.

Spore-Display of Lipases

The compositions of the invention include a spore-display system comprising a spore and a lipase displayed on the spore surface. Lipases catalyze the hydrolysis or synthesis of long chain acylglycerols. Lipase synthesis of esters from glycerol and long chain fatty acids may occur by ligation or formation of any of the various bonds, oxidation, reduction, the addition or deletion of a chemical moiety, or any other change that affects the structure or activity of the molecule. Lipase substrates include, but are not limited to, prochiral or chiral alcohols, carboxylic acid esters, diols, α- and β-hydroxy acids, cyanohydrins, chlorohydrins, diesters, lactones, amines, diamines, amino-alcohols, and α- and β-amino acid derivatives. While the present invention is not bound by any particular mechanism of lipase hydrolysis, a lipase of the invention may hydrolyze the substrate with a nucleophilic attack that liberates an alcohol and a fatty acid. Lipases tend to exhibit high substrate and product selectivity but also possess broad substrate specificity. Lipases are also defined as carboxylesterases acting on emulsified substrates and as carboxylesterases that catalyze hydrolysis of long chain acylglycerols. See also Jaeger, et al. (1998) Trends in Biotech. 16:396-403.

Lipases are characterized by an α/β-hydrolase fold. The lipase core is composed of a central β-sheet consisting of up to eight different β strands connected by up to six a helices. The lipase active site is formed by a catalytic triad consisting of the amino acids, serine, aspartic or glutamic acid, and histidine. Lipolytic reactions occur at the lipid-water interface, but are not limited to a lipid-water interface. Lipase activity may be assessed by any means known to one of skill in the art, including but not limited to, the monolayer technique or the oil drop technique (Jaeger, et al. (1998) Trends in Biotech. 16:396-403).

Industrial uses of lipases include, but are not limited to, uses in detergents, food ingredients, the pulp and paper industry, flavor development, waste treatment, and industrial synthesis. Spore displayed lipases may be used in these and other industrial applications. In an embodiment of the invention, spores displaying lipases are added to detergents for use in household and industrial laundry and residential dishwashers. Similarly, lipases may be used to treat high fat wastes or fat-containing waste effluents. Currently the extreme conditions of temperature and pH (30-60° C. and pH 10-11) in laundering and washing processes reduce the stability and durability of lipases in detergents. By providing the lipases in a spore display system, the durability of the spore protects the lipase from the harsh conditions and allows reuse of the lipase (Jaeger, et al. (1998) Trends in Biotech. 16:396-403).

Lipases are added to plant and animal derived polyunsaturated fatty acids to enrich the fatty acids. In an embodiment, spore displayed lipases are added directly to food to enrich the foods. In addition, an immobilized lipase is used to synthesize a substitute for cocoa butter. Lipases are also used to develop flavors for dairy, alcohol, chocolate, and candy products. The lipases hydrolyze fat triglycerides to release fatty acids that can be used as flavorants or flavor precursors. In another embodiment spore displayed lipases synthesize food additives (Jaeger, et al. (1998) Trends in Biotech. 16:396-403).

Pitch, comprised of the hydrophobic components of wood include triglycerides and waxes, interferes with pulp and paper manufacture. Therefore, the pitch must be removed. Lipases hydrolyze the triglyceride components of pitch. The compositions of the invention may be used to remove or reduce the pitch concentration in the manufacture of pulp or paper (Jaeger, et al. (1998) Trends in Biotech. 16:396-403).

Organic chemists and chemical manufacturers utilize lipases to catalyze a plethora of chemo-, regio-, and stereoselective transformations. Current industrial products include, but are not limited to, chiral amines and (2R, 3S)-3-(4-methoxyphenyl) methyl glycidate (Jaeger, et al. (1998) Trends in Biotech. 16:396-403). However, industrial applications for lipases have been limited by the difficulty of reusing the lipase and the fragility of lipases. The spore-displayed lipases of the invention address both of these challenges. Details of the analysis of the enzymatic activity of a spore-displayed lipase are provided elsewhere herein.

Other Applications

In one embodiment, the spore system of the present invention can be used to display polypeptides that are useful in molecular engineering processes, such as affinity chromatography. For such applications, the spores themselves, being rigid spheres, would constitute the matrix and the displayed protein would be the active chemical constituent. In such an application, the spores of the present invention provide a means for purification of the reaction solution because, in addition to settling to the bottom of a reaction vessel, they may also be removed by centrifugation. In such a way, a spore display system displaying restriction enzymes could be added to a solution containing DNA, incubated for an appropriate reaction time, and then the spores and the attached restriction enzymes could be removed by centrifugation, thereby obviating the need for time-consuming and potentially destructive enzyme inactivation steps such as heat inactivation, organic extraction, or detergent treatment of the DNA and/or solution.

In another embodiment, the spore display systems of the invention may be used in a capture system, such as biotinylated capture system. Currently, biotinylated capture systems rely on purified avidin bound to a solid matrix such as beads. For example, the spore of the invention may be modified to display avidin, which has high affinity for biotin. The spore-avidin system may be applied to a complex mixture of molecules containing a small percentage of biotinylated target molecules. The avidin on the spores binds to biotinylated molecules such as nucleic acid (e.g., DNA), polypeptides, antigens, or other molecules as described above, linking biotinylated molecules to the spores. When the spores are removed from the mixture, the spores bring the biotinylated target molecule with them. In the spore system of the current invention, the spore synthesizes the avidin and retains it on the spore so that the spore functions as the bead or solid support. Thus, the spore system of the invention eliminates the need for purification of avidin and the linking step in which avidin is attached to a bead.

Spore display systems can be used in conjunction with Fluorescent Activated Cell Sorting (FACS) as a primary assay for proteins having a desired structural or sequence epitope. Variants of these epitopes can be identified and segregated rapidly, also. For example, spores that have been transformed with a library of variants are reacted with antibodies conjugated with fluorophores. The labeled antibodies bind structurally relevant epitopes on the spore. The spores are interrogated by laser beams and segregated by the magnetic field of the FACS instrument. Thus, the invention allows high throughput analysis of multiple variants.

Additional embodiments of the invention includes spore systems, spore display systems, spore encapsulate systems, and methods and compositions thereof having as screening agents for allergens and diagnoses related to allergies. For example, in one aspect, the invention provides spore systems and spore display systems comprising spores that display, present, or provide one or more of a wide variety of allergen polypeptides or peptide fragments or variants thereof and diagnostic methods using such spore. Spores with surfaces decorated with such polypeptides or peptide fragments or allergen variants can be screened in a standard binding assay to determine those allergen polypeptide, fragments, or variants that do bind or do not bind a subject's allergen-specific immunoglobulin, e.g., IgE. Determination of those allergens and variants thereof to which subject (e.g., patient) has or does not have an allergic response is of great assistance in developing immunotherapeutic or immunoprophylactic methods for treating allergic diseases. One of ordinary skill in the art will readily understand how to apply such information in established allergy treatment protocols. A wide variety of known allergen polypeptides, fragments thereof, and variants thereof, including recombinant or mutated versions of these, can be used for display, expression, or presentation on spore system or spore display system of the invention.

Further embodiments of the invention include spore systems and compositions thereof having antipathogenic activity and methods of using such systems and compositions to enhance immune responses against pathogens. Pathogen attack on plants results in tremendous economic challenges for farmers throughout the world. Millions of dollars are spent annually to reduce the impact of plant pathogens on agricultural crops. Pathogens include, but are not limited to, insects, fungi, nematodes, bacteria, and viruses. The spore display systems of the invention may be used to display and deliver peptides, polypeptides, proteins, carbohydrates, or nucleotides with anti-pathogenic activity to plants. The spore display system may display one or more anti-pathogenic compounds that target one or more pathogens of interest.

By “anti-pathogenic” is intended that the composition has activity against one or more pathogens and thus is capable of suppressing, killing, and/or controlling the invading pathogenic organism. An antipathogenic composition of the invention reduces the disease symptoms resulting from microbial pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect organisms, particularly plants, from disease, particularly those diseases that are caused by invading pathogens. Assays that measure antipathogenic activity are commonly known in the art, as are methods to quantify disease resistance in plants following pathogen infection. See, for example, U.S. Pat. No. 5,614,395, herein incorporated by reference in its entirety.

One of skill in the art knows of a variety of compounds with antipathogenic activity including, but not limited to, defensins, Bt toxins, fungicides, insecticides, thionins, antibodies, lipid transfer proteins, virus particles, lectins, and lipoxidases. Genes encoding disease resistance traits include, but are not limited to, detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Delivery of the spore system of the invention to the environment of the plant may be accomplished through a variety of methods including, but not limited to, those described elsewhere herein. In some embodiments, methods of applying an active ingredient of the present invention or an agrochemical composition of the present invention (which contains at least one spore system of the present invention) are foliar application, seed coating, and soil application.

The compositions of the invention can be formulated with an acceptable carrier into a composition(s) that is, for example, a suspension, a solution, an emulsion, a dusting powder, a dispersible granule, a wettable powder, an emulsifiable concentrate, an aerosol, an impregnated granule, an adjuvant, a coatable paste, and also encapsulations in, for example, polymer substances. Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors or other preparations that influence plant growth.

Kits

The present invention also provides kits including the spore systems, modified spores, recombinant spores, vaccines, compositions, and methods of the invention. Kits of the invention optionally comprise at least one of the following of the invention: (1) spore systems, modified spores, recombinant spores, vaccines, compositions, or components thereof or combinations thereof as described herein; (2) at least one kit component comprising a spore system, modified spore, recombinant spore; (3) instructions for practicing any method described herein, including a therapeutic or prophylactic methods, instructions for using any component identified in (2) or any vaccine or composition comprising at least one spore system, modified spore, or recombinant spore; (4) a container for holding said at least one such spore system, modified spore, recombinant spore, vaccine, or composition; and (5) packaging materials.

In a further aspect, the present invention provides for the use of any spore system, modified spore, recombinant spore, vaccine, composition, or kit described above and herein, for the practice of any method or assay described herein, and/or for the use of any spore system, modified spore, recombinant spore, vaccine composition, or kit any assay or method described herein.

The following examples are offered not by way of limitation but rather by way of illustration.

### EXAMPLES

### Example 1

### Development of a Vaccine for Yersinia pestis

Nucleotide sequence clones are obtained for Yersinia pestis V-antigen from each of 29 strains of Yersinia pestis, representing the global diversity of Yersinia pestis at the V-antigen locus. The V antigen from Yersinia pestis is a 37 kDa virulence factor that induces protective immune responses against Yersinia and is currently being evaluated as a subunit vaccine (Brubaker (1991) Current Investigations of the Microbiology of Yersinae, 12: 127). The V-antigen alone is not toxic, but Y. pestis isolates that lack the V-antigen are avirulent. The Yersinia V-antigen has been successfully produced in E. coli by several groups (Leary et al. (1995) Infect. Immun. 3: 2854). These clones are ligated into a set of expression vectors comprising the promoter of the gene encoding the Bacillus subtilis spore coat protein cotC. The expression vectors further comprise linker sequences in order to easily ligate an assortment of clones into the vectors so as to be operably linked with the cotC full-length nucleotide sequence or fragment thereof. The set of expression vectors containing the clones (hereinafter, “substrate plasmids”) is introduced into a host strain of Bacillus subtilis by, e.g., electroporation, natural competence, or other method of nucleic acid transfer. Following transfer of nucleic acid into the cell, the pool of Bacillus subtilis is allowed to replicate briefly and then is subjected to sporulation conditions so that the resulting spores display an assortment of Yersinia pestis V-antigens on their surface. These spores are screened using a competition assay with immobilized antibodies from a rabbit exposed to a mixture of cell extracts from a variety of Yersinia pestis strains; those spores displaying antigens which bind to the antibodies are selected. Spores identified by this method are germinated and grown under selection to propagate the candidate substrate plasmids. These plasmids are then isolated and used as substrate plasmids for a second round of screening and selection. This process is reiterated from one to five more times. For example, the selected spores are germinated and plasmids are isolated therefrom. These plasmids are re-introduced into the Bacillus subtilis, and sporulation is induced and spores are screened using the competition assay or another assay. The process is repeated until substrate plasmids having the optimum properties (e.g., those demonstrating stronger binding to the immobilized antibodies) are identified and obtained. Bacillus subtilis containing these plasmids are then propagated, sporulated, and used to vaccinate mice. Vaccination is performed by, e.g., aerosolization of the spores and delivery with nebulizers placed over the nose of the mouse. After allowing sufficient time for an appropriate immune response, the immunized mice are then challenged with a mixture of Yersinia pestis strains to assess the efficacy of the vaccine.

### Example 2

### Display of an Antigen from Yersinia pestis on B. subtilis Spores

B. subtilis cotC was cloned under the control of its own promoter into an expression vector or cassette that contains both gram positive and gram negative origins of replication. A linker consisting of the HA11 epitope and restriction enzyme sites was engineered into cotC between the codons encoding amino acids 27 and 28 (SEQ ID NO: 1). The inserted sequence is amino acid residues 28-47 of the polypeptide encoded by SEQ ID NO: 1. The HA11 epitope is residues 32-43 of the polypeptide encoded by SEQ ID NO: 1. The nucleotide sequence encoding the wild type V antigen from Y. pestis, the causative agent of bubonic plague, was cloned in-frame into the PstI site in the linker. This results in a construct encoding cotC, inserted into which is V antigen fused to the HA11 epitope. In this example the HA11 epitope is used to simplify detection and analysis. Monoclonal antibody to HA11 was raised against the twelve amino acid peptide, and it recognizes a 9 amino acid influenza hemagglutinin (HA) epitope, which has been used extensively as a general epitope tag in expression vectors. The extreme specificity of this antibody allows unambiguous identification and quantitative analysis of the tagged protein. The monoclonal antibody HA11 was purchased from Covance and used according to manufacturer's instructions for the particular assay. The nucleic acid sequence encoding the HA epitopic peptide sequence (either the twelve amino acid sequence or the nine amino acid sequence) was engineered to include two sets restriction sites downstream (BamH I and PST I) and upstream (Kpn I and X ba I) of the epitope sequence for subsequent cloning.

Initial gene construction was done in E. coli. The resultant plasmid was then transformed into B. subtilis and B. subtilis was induced to sporulate. Spores were purified by density gradient centrifugation and washed extensively. Spores were analyzed for localization of V antigen to the spore coat by extraction of the spore coat and analysis by Western blot. Bands of the expected size in spore coat extracts, but not in pellets confirmed localization of recombinant V antigen to the spore outer coat. Spores were then analyzed by flow cytometry using antibodies that recognized either V antigen or HA11. Spores were incubated on ice for 1 hour with the primary mouse monoclonal antibody diluted 1:50 in Dulbecco's Phosphate Buffered Saline with 3% fetal bovine serum (DPBS-FBS). They were washed 3 times in DPBS-FBS, followed by incubation on ice for 1 hour with the secondary phycoerythrin (PE)-labeled goat anti-mouse antibody diluted 1:100. Spores were washed 4 times in DPBS-FBS and analyzed using a FACSCalibur flow cytomoter (Becton Dickinson, San Jose, Calif.) (see FIG. 4) according to the manufacturer's instructions and using known procedures (see, e.g., Colligan and Rapley and Walker, both supra; DeMaio, A., Protein Blotting: a practical approach, ed. B S Dunbar, Oxford Univ. Press 1024,1994; Kolodziej, P.A., et al., Meth. Enzymol. 194:508-519 (1991); and Field, J. et al., Mol. Cell. Biol. 8:2159-2165 (1988)). Greater than 90% of recombinant spores displayed HA11 and V antigen on the surface.

### Example 3

### Spore ELISA to Calculate the Antigen Concentration in a Vaccine Dose

There are many ways to use the ELISA format to ascertain what results the combination of display strain and antigen deliver. ELISAs can be used to determine the anti-spore response, spore concentration, and response of mice to specific antigens displayed on the spore. One possible format of the spore ELISA is described herein.

Control rows in 96 well plates were coated with a dilution series of known amounts of purified protein. Test wells were incubated with 50 μl of 1.8×107 spores/ml in PBS overnight at 4° C. The following day, the spore liquid was removed from the wells. The aspirated liquid may be used to determine spore counts, and the residual spore count is used to determine the number of spores bound to the plate. The plate was blotted with a towel. The plate was washed four times with PBS-Tween by hand. 200 μl of PBS-Tween containing 3% blocking agent (e.g. milk) was added to each well. The blocking agent and plate were incubated together for 1 hour at 37° C.

While the assay plates were incubating with the blocking agents, appropriate dilutions of the antisera were made in 100 μl 3% milk+PBS+Tween.

The assay plate was washed four times with PBS+Tween. 50 μl of 3% milk+PBS+Tween were added to wells of rows B-H. A previously titrated dilution of specific monoclonal antibody or polyclonal sera specific to the recombinant antigen was added to row A. 50 μl of the diluted sera or antibody was transferred from row A to row B. The serial 2 fold dilutions were continued down the plate. The assay plate was incubated for one hour at 25° C.

The assay plate was incubated with the diluted sera for one hour at 25° C. The assay plate was washed four times with PBS +Tween. 50 μl diluted conjugated anti-sera (e.g. anti-mouse IgG-horseradish peroxidase (HRP)) was added to each well. The conjugated anti-sera was incubated with the assay plate for one hour at 25° C. The plate was washed four times with PBS+Tween. 100 μl of a tetramethylbenzidine (TMB) substrate:peroxide mixture (1:1) was added to each well (J Immunol Methods Jan. 13, 2000;233(1-2):47-56). The A450 of each well was determined. The concentration of antigen was determined. Using the spore concentration and antigen concentration, the number of moles of antigen delivered (e.g., micrograms) per spore was determined.

### Example 4

### Immunological Response to Spore Inoculation

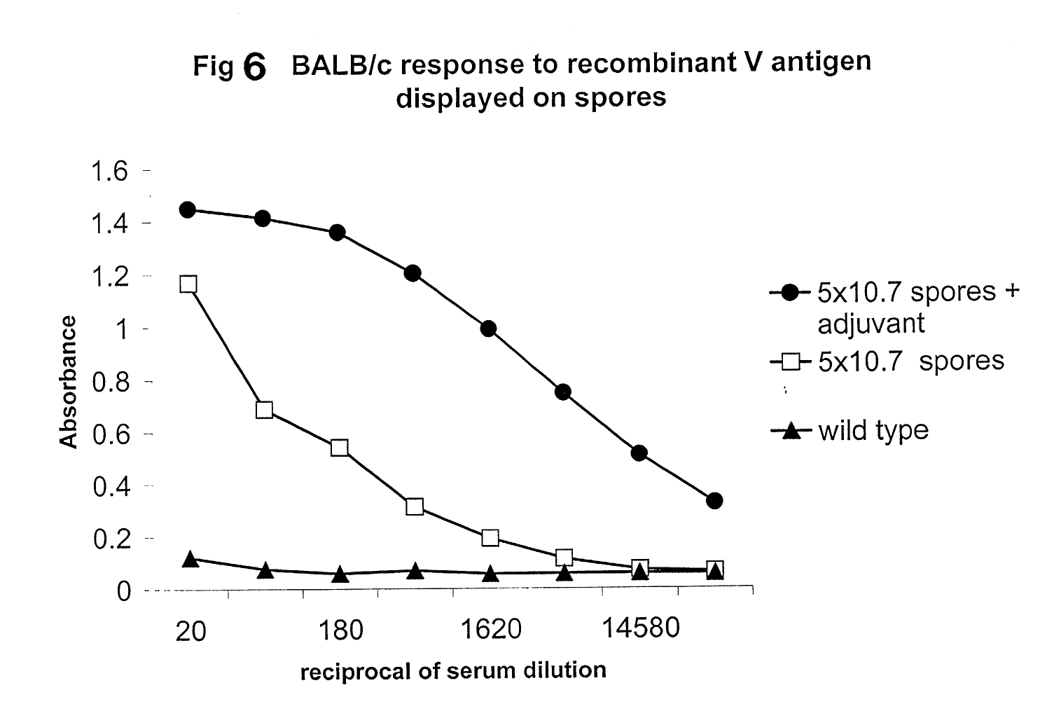

Inoculation of mice with modified or recombinant spores was used to test the ability of the recombinant spores to activate the immune response. Three mice strains, BALB/c, C57B1, and Swiss Webster mice were selected for the analysis. The spores were prepared to display the Y. pestis V antigen fused to cotC on the spore surface. A standard injection schedule was followed: Day 1, subcutaneous prime; Day 21, intraperitoneal boost; Day 35, intraperitoneal boost; and Day 45, terminal bleed. The subcutaneous injections were delivered under the fur behind the neck, and the intraperitoneal injects between the skin and peritoneal cavity near the hind leg. ELISAs were performed on the interim and terminal bleeds to track the antibody titer (see FIG. 6).

### Example 5

### Adjuvant Effect of the Spores

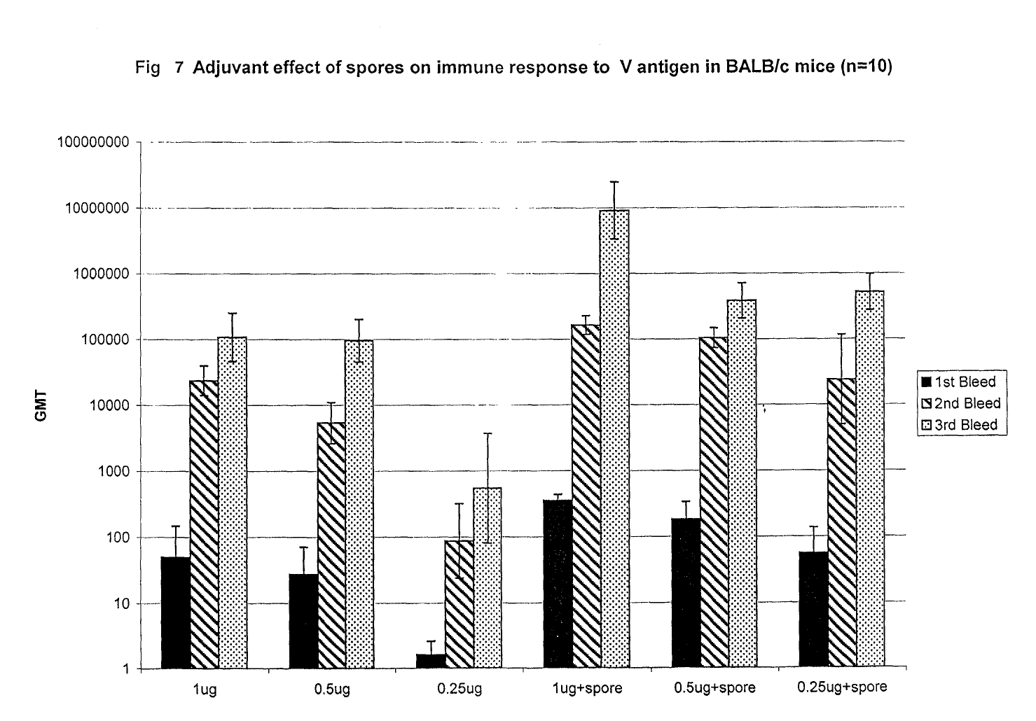

Spores from B. subtilis were tested to determine if the spores have an adjuvant effect, such as, e.g., enhancing an immune response when delivered to a subject with an immunogenic polypeptide. In this example, the specific immunological response of mice to spores and V-antigen mixed together was compared to the specific immunological response of mice to the V-antigen protein alone. 1 μg, 0.5 μg or 0.25 μg of purified recombinant V-antigen was mixed with 5×10⁸ non-recombinant B. subtilis spores or used alone. The three V-antigen protein/spore mixtures and three amounts of V-antigen protein were injected intraperitoneally into separate groups of mice (10 mice in each group), at day 1, day 21, and day 35. Mice were bled on days 10, 21, and 45. Serum was analyzed for specific anti-V-antigen immunoglobulins by an indirect ELISA using standard procedures as described above. The geometric mean antibody titer (GMT) for each group of 10 mice is indicated in FIG. 7. The presence of spores in the inoculum increased the antibody titer between 10-fold and 1000-fold, depending on the amount of protein inoculated. The data suggest spores act to augment a specific immune response to an immunogenic polypeptide, such as V-antigen protein.

### Example 6

### Spore Display of Lipase 396 and Enzymatic Assays Thereof

Two different expression constructs comprising lipase 396 were created. In one expression construct (Clone 16), the lipase 396 gene (SEQ ID NO:2) is inserted in the CotC sequence between the codons encoding amino acids 27 and 28. Clone 16 expresses a fusion protein with fragments of CotC located N-terminally and C-terminally to the lipase 396 protein. In the second expression construct (Clone 19), the lipase 396 gene operably linked to a translational termination region were inserted in the CotC sequence between the codons encoding amino acids 27 and 28 of CotC. Clone 19 expresses a fusion protein of the N-terminal 27 amino acids of CotC with lipase 396. The translational termination region stops translation and prevents translation of the C-terminal portion of CotC.

The expression constructs were transformed into B. subtilis. The B. subtilis cells were induced to sporulate by nutrient deprivation. During sporulation, the fusion proteins of clone 16 and clone 19 were generated and incorporated into the spore coat.

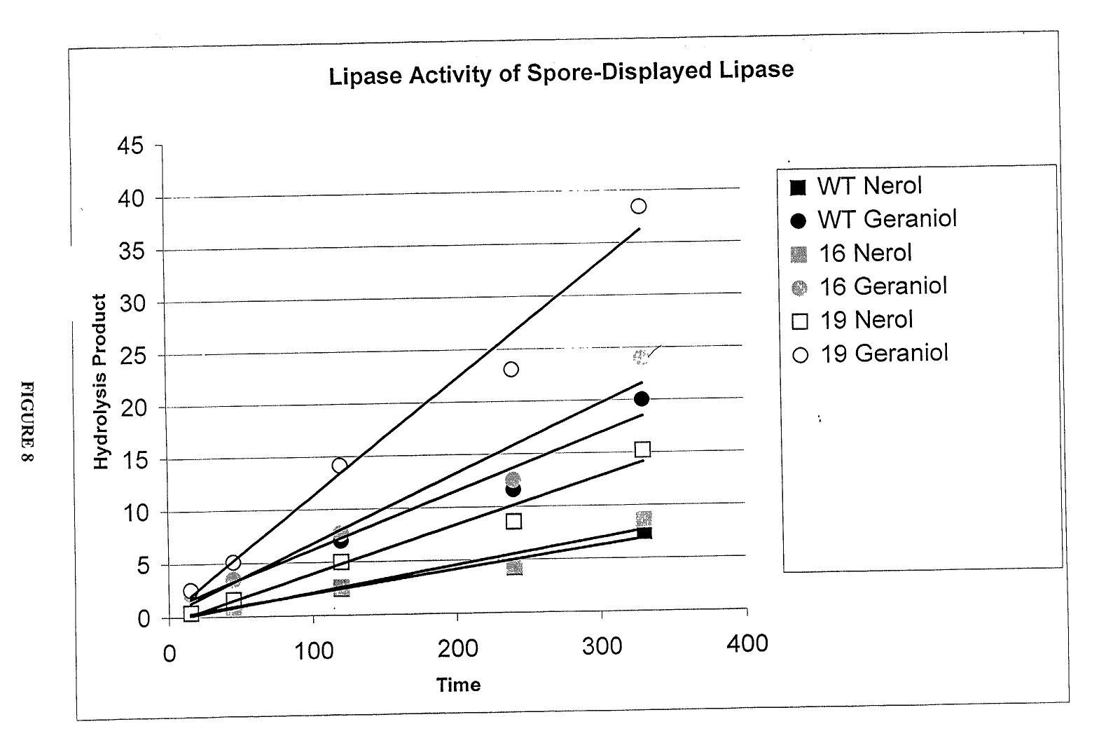

Stock spore samples were normalized to contain the same amount of spores per unit volume. Reaction mixtures containing 60 μl 10 mM morpholine acetate buffer (pH 7.4) and 2 μl DMSO containing substrate (75 mM nerolbutyrate or 75 mM geranioldeuterobutyrate) were prepared in 700 pH glass vials. A reaction vial for each time point was prepared for each clone. The reactions were initiated by the addition of 40 μl of the well-suspended, normalized spore samples. The reactions were incubated with vigorous mixing for 15, 45, 120, or 240 minutes. The reactions were quenched with CHCl₃ at the indicated time. The quenching solution contained geranyl acetone as an internal standard for analysis of product formation. Activity of the lipase enzymes towards substrates were determined by gas chromatography/mass spectroscopy to measure the amount of nerol or geraniol formed. Results of this assay are indicated in FIG. 8.

### Example 7

### Delivery of Laccase to Contaminated Pulp Mill Effluent for Bioremediation

A gene encoding a laccase enzyme is cloned into an expression vector containing a promoter and/or gene that direct assembly, display, and/or incorporation of the laccase enzyme within or on the outer coat of the bacterial spore. This expression vector is transformed into a strain of Bacillus subtilis, which is then sporulated. The resulting spores display laccase enzyme on their outer spore coat. These spores are then added to aqueous bleached kraft pulp mill effluents containing pentachlorophenol. The laccase activity provided by the spores removes free pentachlorophenol from the aqueous solution, primarily by polymerization. Polymerized products, having a high molecular weight, are effectively mineralized but may also be removed by filtration by virtue of their size.

### Example 8

### Vaccination of Aquatic Organisms

A gene encoding an antigen such as the glycoprotein from infectious hematopoietic necrosis virus is cloned into an expression vector containing a promoter and/or gene that direct assembly, display, and/or incorporation of the antigen within or on the outer coat of the bacterial spore. This expression vector is transformed into a strain of Bacillus subtilis, which is then sporulated. The resulting spores display the antigen on their outer spore coat. These spores are then added to ponds containing Rainbow Trout or other aquaculture species.

### Example 9

### Comparison of Oral and Nasal Immunization Methods

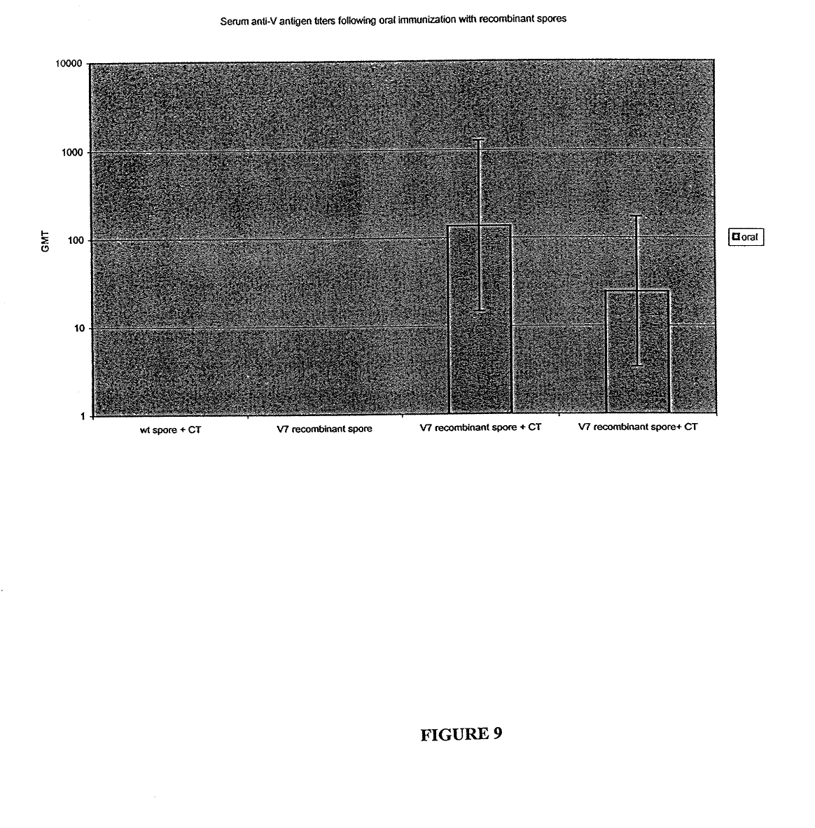

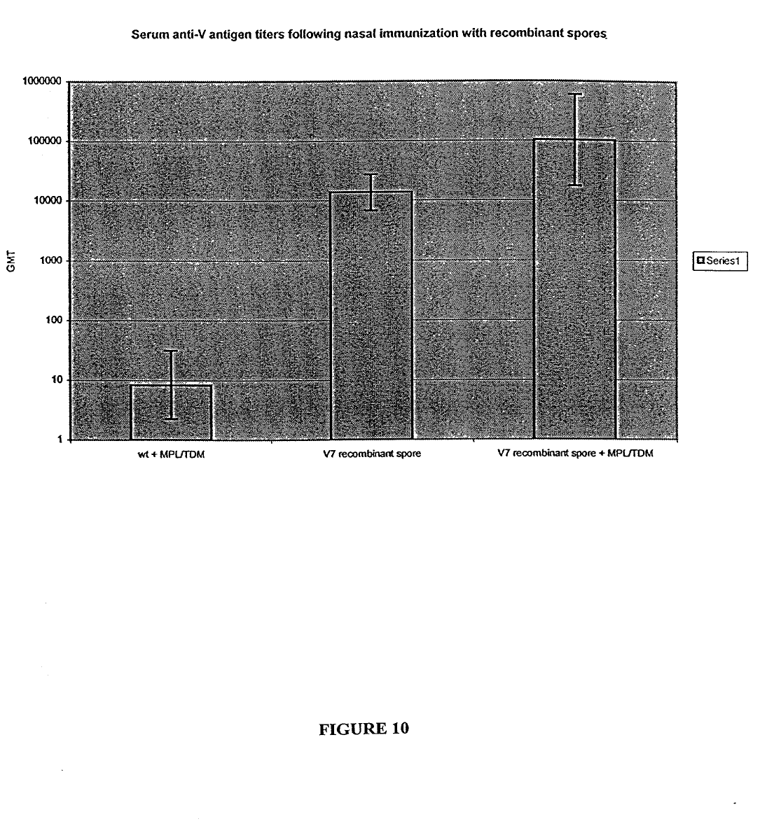

C57BL6 mice (n—5 per group) were inoculated with wildtype (wt) Bacillus subtilis spores or recombinant spores displaying V antigen on the surface as a fusion protein with CotC (V7). Mice were inoculated orally at day 1 with 5×10⁸ spores, then boosted orally such populations of spores at 3 weeks and 5 weeks. Mice were inoculated with and without V. cholerae (cholera) toxin subunit B (CT) as an oral adjuvant. Sera was collected from the mice 10 days after the final boost, and assayed by a standard ELISA for anti-V antigen immunoglobulin. Antibody titers were calculated as the reciprocal of the dilution at which the serum titrated to two times background, and expressed as geometric mean titers (GMT). V. cholerae toxin subunit B acted as an adjuvant capable of enhancing mucosal immunity and oral delivery of recombinant spores displaying the V antigen. See FIG. 9. C57BL6 mice (n=5 per group) were inoculated with wildtype (wt) Bacillus subtilis spores or recombinant spores displaying V antigen on the surface as a fusion protein with CotC (V7). Mice were inoculated nasally at day 1 with 5×10⁸ spores, then boosted nasally at 3 weeks and 5 weeks. Spores were inoculated with and without MPL/TDM as a nasal adjuvant. Sera was collected 10 days after the final boost, and assayed by a standard ELISA for anti-V antigen immunoglobulin. Titers were calculated as the reciprocal of the dilution at which the serum titrated to two times background, and expressed as geometric mean titers (GMT). See FIG. 10.

Serum immunoglobulin G (IgG) titers against V antigen were obtained when recombinant B. subtilis spores were applied either orally or nasally in the presence of the appropriate adjuvant. Nasal application resulted in 1000-fold higher specific titers than did oral application. The titers obtained using nasal application gave titers similar in magnitude to that obtained in experiments when mice were immunized subcutaneously and intraperitoneally (data not shown). A high titer of serum antibodies to Yersinia pestis V antigen can be obtained by nasally administering spores displaying recombinant V antigen. It has also been demonstrated that nasal administration of these spores induces specific IgA, detectable in the feces, indicating that mucosal immunity is obtained.

Nasal and oral vaccine formats/immunization methods comprise, e.g., co-administration or consecutive administration of spore systems displaying an antigen or antigenic fragment of interest and cholera toxin subunit B as an adjuvant. Such vaccine formats/immunization methods comprise, e.g., co-administration or consecutive administration of spore systems displaying the antigen or fragment thereof and cholera toxin subunit B as an adjuvant. In an alternative fomat, heat labile toxin from E. coli toxins can be used in place of cholera toxin subunit as an adjuvant to enhance mucosal immunity and oral delivery of recombinant spores displaying the antigen of interest. Vaccine compositions for such oral and nasal vaccines comprise spore systems of the invention displaying the antigen or antigenic fragment of interest. If desired, the composition may further comprise a carrier may be included. In an alternative format, heat labile toxin from E. coli toxins can be used in place of cholera toxin subunit as an adjuvant to enhance mucosal immunity and oral delivery of recombinant spores.

### Example 10

### Determination of Adjuvant Effect of Spores on DNA Vaccination

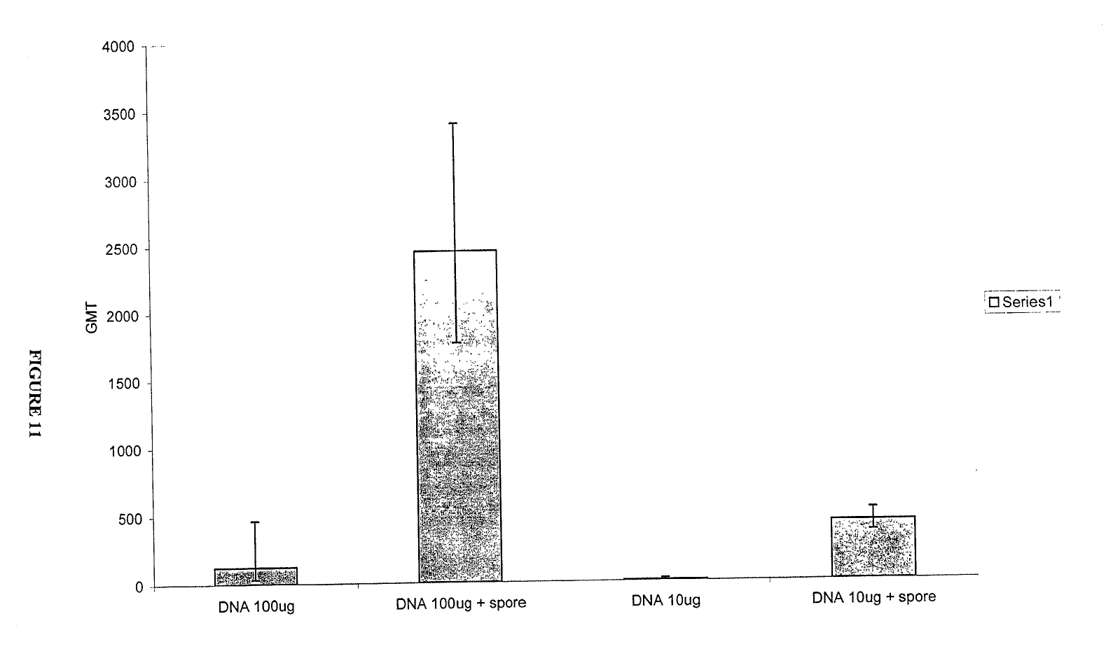

The ability of B. subtilis. spores to enhance the immune response to a DNA vaccine was tested by co-administering a plasmid encoding the Hepatitis B surface antigen with B. subtilis spores. Groups of 5 C57BL.6 mice were inoculated intramuscularly with either 10 μg or 100 μg of plasmid, with or without 5×10⁸ non-recombinant B. subtilis spores. Mice were boosted with inoculations at 3 weeks, then again at 5 weeks, and serum was collected 10 days following the final boost. Serum was tested for anti-surface antigen immunoglobulins using a standard indirect ELISA format. The endpoint antibody titer was calculated as the dilution corresponding to twice background. Results are shown in FIG. 11. Data was expressed as the geometric mean titer (GMT) of five samples.

Co-administration of B. subtilis spores with 100 μg DNA resulted in a 20-fold increase in measured specific immunoglobulin. If the dose of DNA was lowered to 10 μg, at which an immune response was barely detectable, co-administration of B. subtilis spores resulted in greater than 60-fold increase in specific immunoglobulin. The sera were also tested in a commercial anti-HepB kit, used to determine if protective levels are obtained (>10 mIU/mL is considered protective). The only group of mice obtaining levels correlated with protection were the mice injected with 100 μg DNA and spores.

### Example 11

### Surface Display of Multiple Epitopes in Fusion with Spore Coat Proteins

Double recombinant spores were constructed as follows: a nucleotide sequence encoding the HA11 epitope flanked by restriction enzyme sites (NotI/KpnI upstream and BamHI/PstI downstream) was inserted after the codon encoding amino acid 27 in the cotC gene. A nucleotide sequence encoding the c-myc epitope and restriction enzyme sites (DraIII and PstI) was inserted at the C-terminal of the cotV gene. Both recombinant genes were cloned under control of their own copy of the sporulation-specific cotC promoter. A plasmid was constructed containing both recombinant genes, and gram positive and gram negative origins of replication. Genetic manipulations were done in E. coli by standard procedures (Sambrook et al). The plasmid was then transformed into B. subtilis, and recombinant Bacilli induced to sporulate. Spores were purified by density gradient centrifugation and extensive washing.

Spores were stained with fluorescently labeled antibodies in the following order: 1) monoclonal anti-c-myc; 2) phycoerythrin (PE)-labeled goat anti mouse antibody; 3) polyclonal anti-HA11; 4) fluorescein isothiocyanate (FITC)-labeled goat anti-rabbit antibody. Staining was done by resuspending the spores in antibody diluted in Dulbecco's Phosphate Buffered Saline with 3% fetal bovine serum (DPBS-FBS). Spores were incubated on ice with the relevant antibody for 1 hour, then washed 3 times in ice-cold DPBS-FBS, followed by staining with subsequent antibodies. Spores were analyzed using a FACScalibur flow cytomoter (Becton Dickinson; San Jose, Calif.) using FL1-H to measure FITC and FL2-H to measure PE, according to the manufacturer's recommendations. The staining concentration was determined for each labeled protein to provide a maximal Mean Fluorescence Intensity (MFI) and minimal background signal.

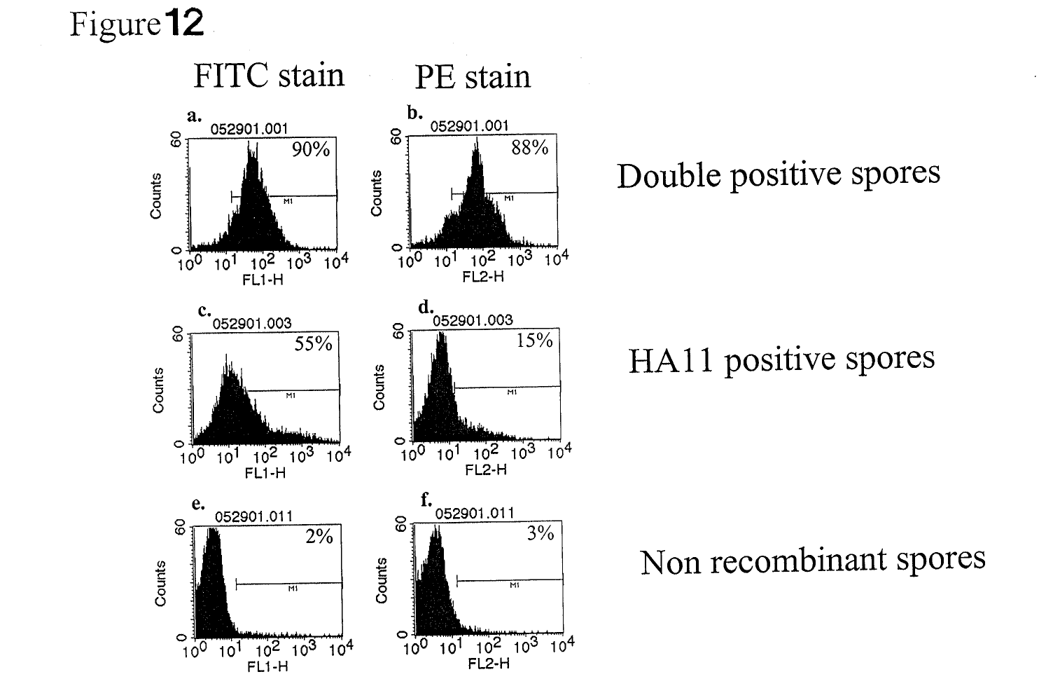

Analysis of double positive spores is shown in FIGS. 12A and 12B. Spores positive only for HA11 are shown in 12C and 12D. Non-recombinant spores displaying neither epitope are shown in 12E and 12F. Spores displaying only HA 11 were constructed by inserting a sequence coding for HA11 linked to the Y. pestis V antigen gene, flanked by restriction enzyme sites, after the codon encoding amino acid 27 in the cotC gene. This gene was placed under the control of the sporulation specific cotC promoter and cloned into the plasmid used above containing both gram positive and gram negative origins of replication. The remainder of the procedure was done as described above for double recombinant spores.

The data shows 90% of double positive spores display HA11 on the surface and 88% of double positive spores display c-myc on the surface. This demonstrates that both cotC and cotV may be used for surface display of epitopes and polypeptides.

### Example 12

### Surface Display of Multiple Polypeptides Fused to Spore Coat Proteins

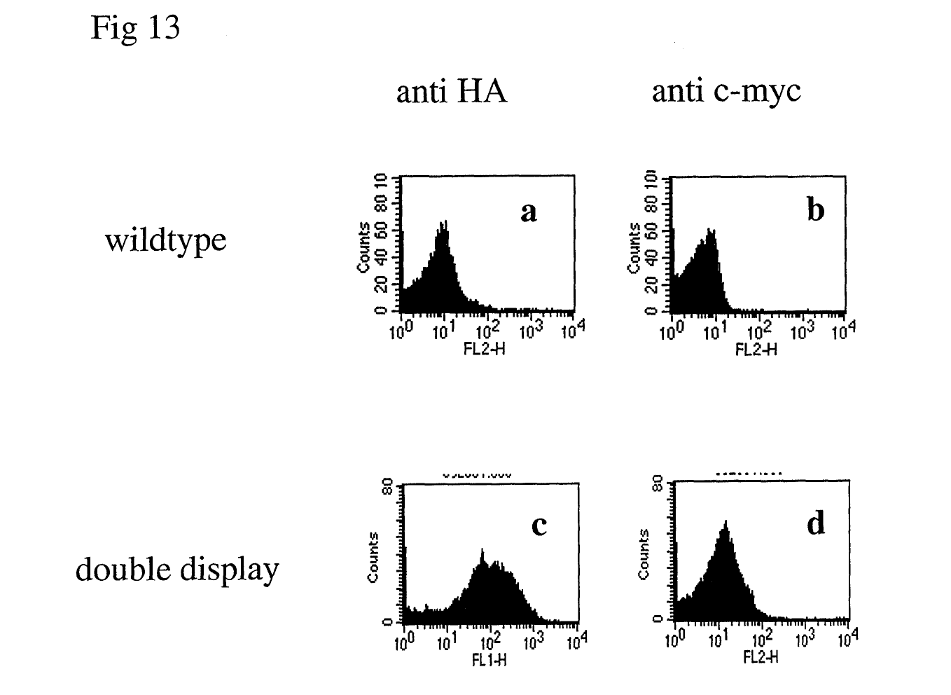

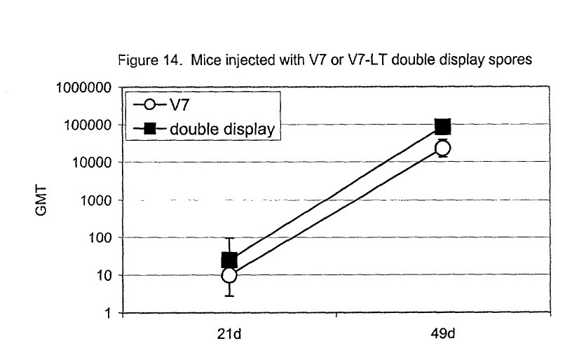

Double recombinant spores were constructed as described in Example 11. The Heat Labile toxin subunit B (LT) from E. coli was cloned next to the c-myc epitope in cotV (by attaching or inserting the LT nucleic acid sequence adjacent next to the nucleic acid sequence encoding c-myc in cotC nucleic acid sequence using standard procedures of molecular biology as described in, e.g., Walker and Gaastra, eds. (1983), Techniques in Molecular Biology (MacMillan Publishing Company, New York); Sambrook et al. (1989) Molecular Cloning: A Laboratory Manual (2^{nd} edition) (Cold Spring Harbor, New York), and V antigen was similarly cloned next to the HA11 epitope in cotC. For example, the nucleic acid sequence encoding LT from E. coli was cloned next to the nucleic acid sequence encoding the c-myc epitope in the cotV gene. The LT subunit can function as a mucosal adjuvant. Analysis of double positive recombinant spores is shown in FIG. 13. This demonstrates that polypeptides fused to cotC and cotV can be simultaneously displayed on the surface of B. subtilis spores. These spores (5×10⁸) were injected into BALB/c mice (n=5) intraperitoneally on days 1, 21 and 35. Phosphate-buffered saline (PBS) was used as the carrier for spores. Serum was collected from the mice at days 21 and 49 and assayed for anti-V antigen immunoglobulins using an indirect ELISA. Results expressed as geometric mean titer (GMT) are shown in FIG. 14.

### Example 13

### Surface Display of an Adjuvant

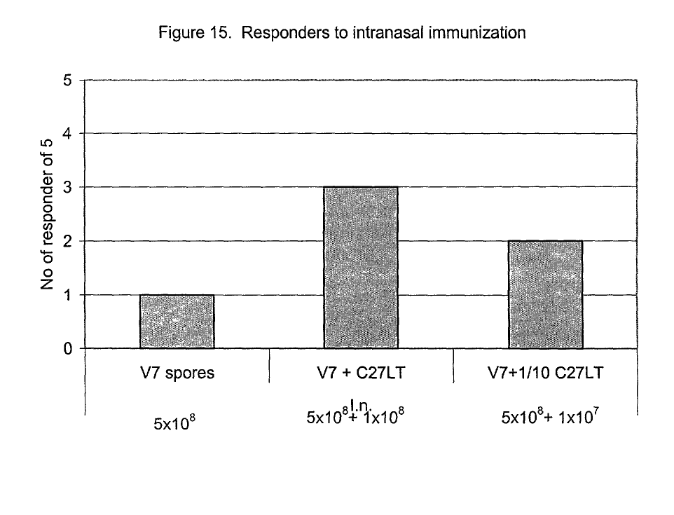

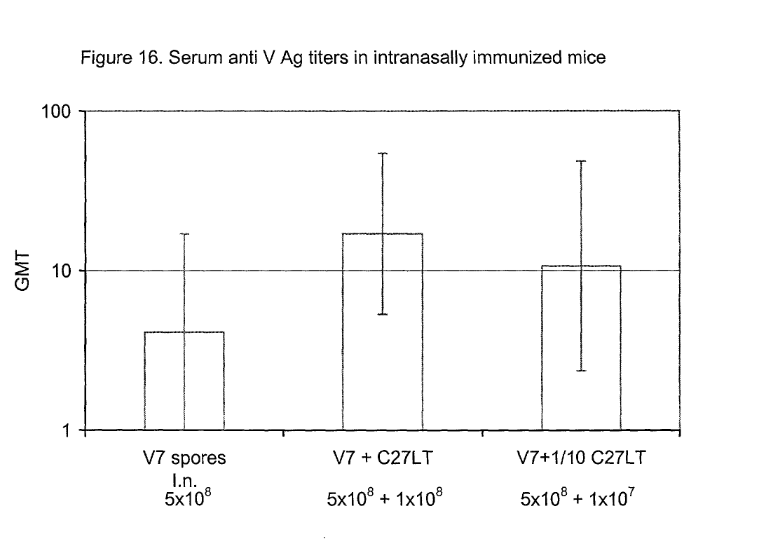

The gene encoding Heat labile toxin subunit B from E. coli (LT) was cloned into the nucleic acid sequence encoding cotC protein after the codon encoding amino acid 27 of cotC, and separately into the C-terminal position of cotV. Bacilli were sporulated, purified, and analyzed for surface display. Spores (1×10⁸ or 1×10⁷) displaying LT fused to cotC were then mixed with spores displaying V antigen fused to cotC (5×10⁸) and inoculated intranasally into BALB/c mice. In FIGS. 15 and 16, V7+C27LT means 5×10⁸ spores displaying V antigen fused to cotC mixed with 1×10⁸ spores displaying LT fused to cotC. V7+1/10C27LT means 5×10⁸ spores displaying V antigen fused to cotC mixed with 1×10⁷ spores displaying LT fused to cotC. All of these spores were inoculated intranasally into BALB/c mice (n=5) on days 1, 21 and 35 in a volume of 5 ul. Phosphate-buffered saline (PBS) was used as the carrier for the V antigen and/or spores throughout this procedure. Serum was collected on day 49 and assayed for anti-V antigen immunoglobulins using an indirect ELISA. The number of mice responding to this route of intranasal inoculation was increased by the presence of spores displaying the LT (FIG. 15). The serum titers showed a trend towards being increased in those mice exposed to LT spores in addition to V antigen spores, although this did not reach statistical significance in this sample. Titers expressed as geometric mean titer (GMT) are shown in FIG. 16.

### Example 14

### Pathogen challenge of Mice Vaccinated by Injection

The gene encoding V antigen from Yersinia pestis was cloned into the nucleic acid sequence encoding cotC after the nucleic acid codon encoding amino acid 27 of cotC, Bacilli were sporulated, purified and the resulting recombinant spores injected into BALB/c mice as follows. A population of 5×10⁸ or 5×10⁹ recombinant spores (i.e., spores engineered to display V antigen) was injected subcutaneously into each of two groups mice (n=12) on day 1 (5×10⁸ recombinant spores delivered to one group and or 5×10⁹ recombinant spores delivered to the second group), then both groups of mice were boosted by intraperitoneal injection with the population of 5×10⁸ or 5×10⁹ recombinant spores as injected on day 1, intraperitoneally on days 21 and 35 (see third and fourth bars on bar graph labeled “recombinant spores”). As a control, one group of mice (n=12) was injected subcutaneously on day 1 with 10 ug V antigen protein, and one group of mice (n=12) was injected subcutaneously on day 1 with 5 ug V antigen protein; each group of mice was boosted with the same amount of V antigen that had been administered on day 1, on days 21 and 35. PBS was used as the carrier for the V antigen and/or spores throughout this procedure.

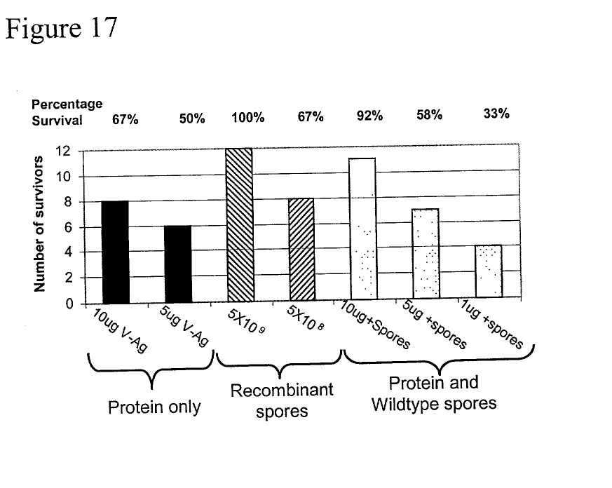

Three groups of mice (n=12) were injected subcutaneously (s.c.) on day 1 with one of the following: 1) 5×10⁷ wildtype (WT) spores (i.e., without displayed antigen) mixed with 10 ug V antigen (free) in PBS; 2) 5×10⁷ WT spores mixed with 5 ug V antigen in PBS; and 3) 5×10⁷ WT spores mixed with 1 ug V antigen in PBS. All 3 groups were boosted by s.c. injection with the same amount of V antigen administered on day 1 (see 5th-7th bars on graph in FIG. 17).

On day 55 mice were challenged with approximately 1000 LD₅₀s of Yersinia pestis, the agent of plague. LD₅₀ is the amount of Y. pestis that was found to kill 50% of a population of mice (e.g., within 14 days of injection of the toxin) as determined by standard procedures using dose titration (e.g., delivering a range of increasing amounts of Y. pestis to separate groups of mice to determine that dose amount that resulted in the death of 50% of the number of mice in a particular group).

Unvaccinated mice were included in the challenge experiment as controls. 14 days after challenge survivors were determined, as shown in FIG. 17. Mice in the 7 groups of mice died between days 4 and 12; all mice were dead by day 12 (data not shown). Mice vaccinated with 3 doses of 5×10⁹ recombinant spores had a 100% survival. All unvaccinated challenged mice died. Thus, these recombinant spores conferred protection in vivo in mice against challenge by Yersinia pestis in a dose-dependent manner.

The 3 groups of mice to which V antigen protein (10 ug, 5 ug, and 1 ug) plus WT spores were administered (see 5^{th}-7^{th} bars on graph in FIG. 17) showed increased survival compared to the two groups of mice to which 10 ug and 5 ug V antigen alone were administered, respectively (see 1^{st} and 2^{nd} bars on graph in FIG. 17); the increased survival rate indicated that the administration of spores had adjuvant effect.

### Example 15

### Pathogen Challenge of Mice Vaccinated by Intranasal Application

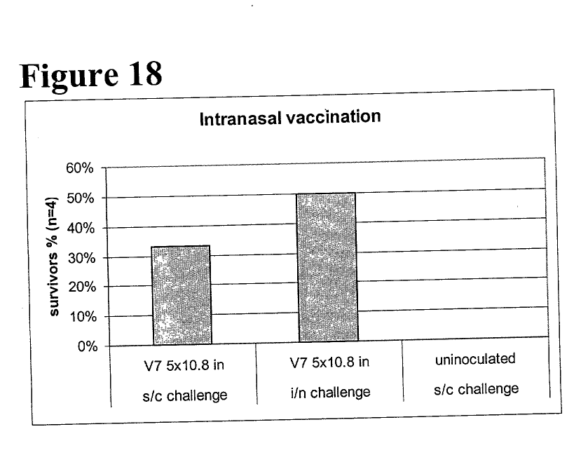

The gene encoding V antigen from Yersinia pestis was cloned into the nucleic acid sequence encoding cotC after the codon encoding amino acid 27 of cotC, Bacilli were sporulated, purified and inoculated into three groups of BALB/c mice (n=4 in each group) as follows. 5×10⁸ recombinant spores (displaying V antigen) per mouse were instilled intranasally in a volume of 25 ul/nostril on day 1. PBS was used as the carrier for spores in intranasal delivery. Mice were boosted in the same manner on days 21 and 35. On day 55 mice were challenged either subcutaneously (s/c) or intranasally (i/n) with approximately 1000 LD₅₀ of Yersinia pestis, the agent of plague. 14 days after challenge survivors were determined, as shown in FIG. 18. The number of mouse survivors challenged with the toxin via each route of administration (subcutaneously and intranasally) is shown.

### Example 16

### Display of an Insecticidal Toxin

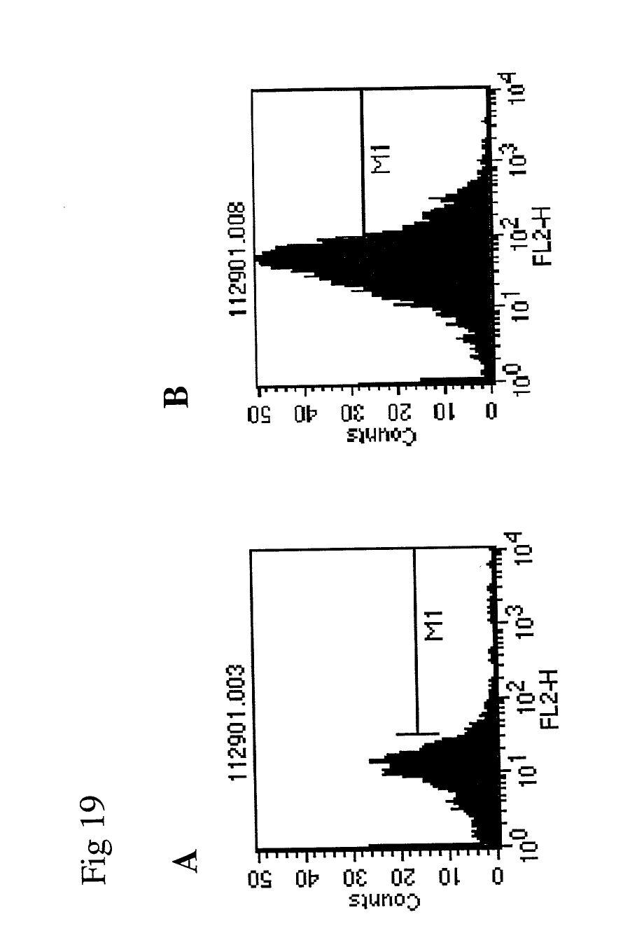

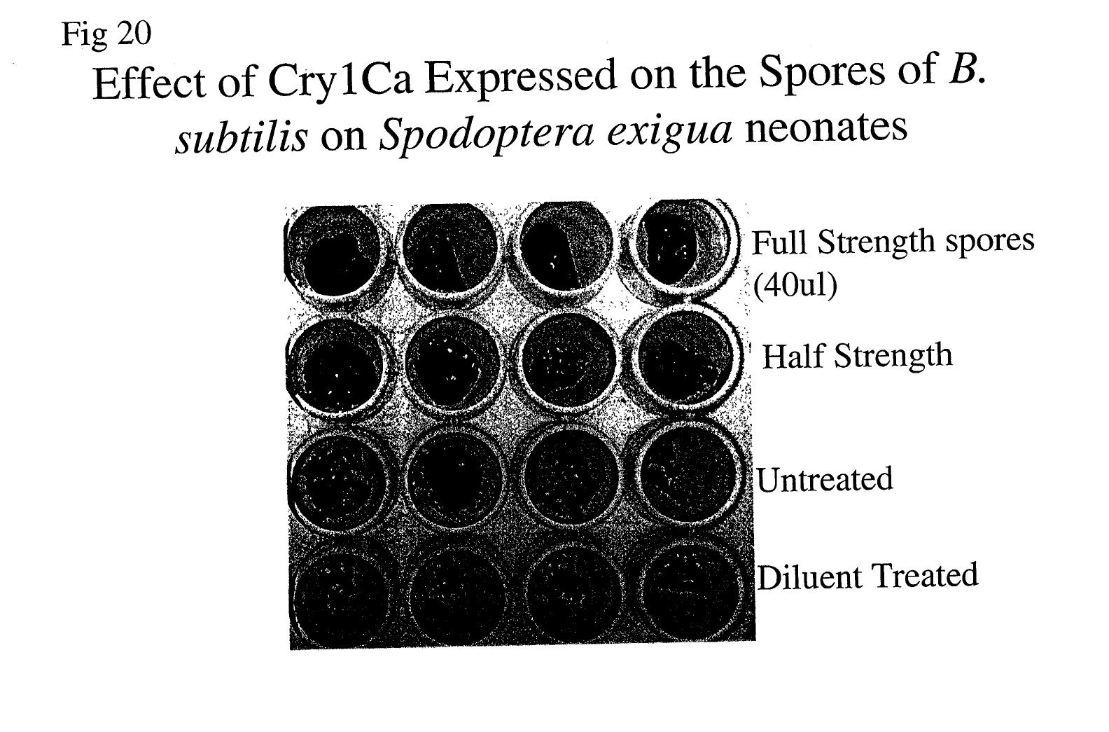

The gene encoding the mature Cry1Ca toxin from Bacillus thuringiensis (BT) was cloned into the nucleic acid sequence encoding cotC-HA11 after the codong encoding amino acid 27 of cotC-HA11, and a terminator was included at the C terminus of the toxin so that the C terminal of cotC was not translated. Bacilli were sporulated, purified, and surface display of BT toxin was confirmed by FACS analysis (FIG. 19). Spores were analyzed in a leaf assay for insecticidal activity (FIG. 20).

### Example 17

### Display on a Non-Germinating Spore

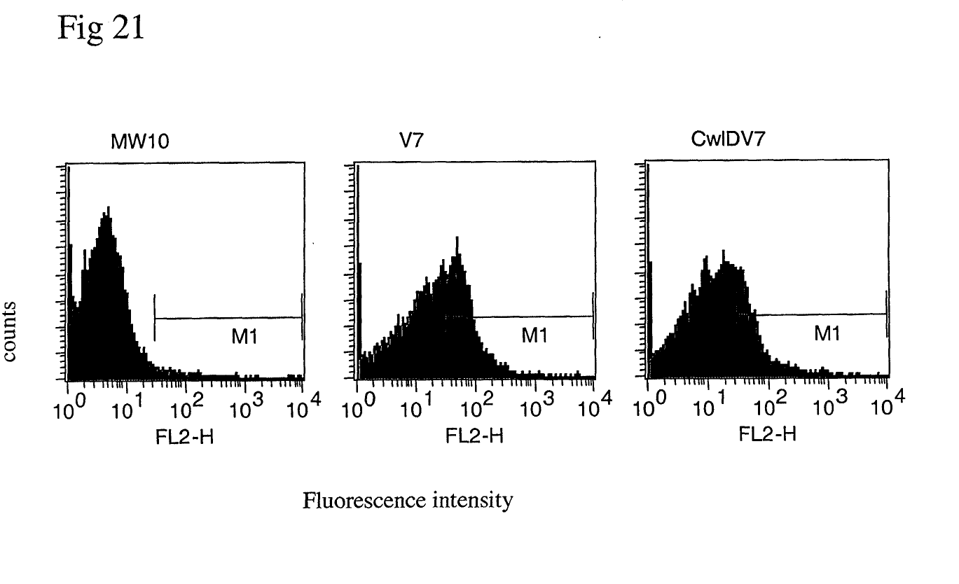

A mutant B. subtilis strain was constructed in which cwlD was mutated to be nonfunctional. cwlD is a Bacillus gene coding for the cell wall lytic enzyme, N-acetylmuramyl amidase (Popham, D et al, J Bacteriology 1999, 181:6205-9). This mutation has the effect of decreasing germination by a factor of 10⁵. The plasmid containing V antigen fused to the aa27 position of cotC was transformed into this strain, Bacilli were sporulated and purified. The non-germinating strain was determined by FACS analysis to display V antigen on the surface (FIG. 21).

### Example 18

### Display of a Multiple Subunit Homomeric or Heteromeric Multimeric Protein

Multiple subunit homomeric and/or heteromeric multimeric proteins, polypeptides, and peptides can be expressed on the surface of spores.

In the homomultimer configuration, a protein that is capable of assembling into a multimer (such as Heat labile toxin (HLT) which in nature assembles itself into a pentamer) is expressed on the spore in at least one of two different formats or in both formats simultaneously. The first expression format comprises a first expression cassette in the spore genome that allows for single copy expression of at least one protein (or polypeptide or peptide) of interest, which is driven by a sporulation specific promoter from a position in the spore genome. In this format, the spore genome is altered to include the nucleotide sequence encoding the protein. This construct is, for example, driven by a cot C promoter and produces a fusion protein of the protein of interest fused to the encoded cot C amino acid sequence (such as, e.g., a fusion protein comprising HLT sequence fused to the encoded cot C coding sequence). The resulting fusion protein is a monomer assembled into/on the spore surface.

The second component of this expression system comprises a plasmid that comprises a nucleic acid sequence and expression control element (e.g., promoter) that encodes at least one of the same protein of interest (or polypeptide or peptide) as described above. This second component may be a second expression cassette. The plasmid is delivered to and/or taken up by the spore or delivered to or included in a composition comprising the recombinant expressing the protein of interest. One of skill in the art would recognize other formats in which the plasmid can be delivered to the spore. The plasmid expresses the protein of interest (e.g., HLT protein), but not as a fusion protein. Rather, the protein is expressed freely from the plasmid in, e.g., soluble form. Expression of the plasmid (e.g., second expression cassette) begins at a slightly later time point than does expression of the protein from the spore genome. The plasmid (second expression cassette) produces protein monomer subunits that assemble onto or become associated with the tethered (fused) monomer subunit expressed on the surface of the spore, thereby forming homomultimeric units (multiple identical or equivalent subunits).

In the heteromeric multimer configuration, the same expression formats as described above for the homomeric multimer are used to produce: 1) at least one protein(s) of interest (or polypeptide or peptide) expressed from the spore genome as a fusion protein fused to, e.g., the amino acid sequence encoded by a cot C gene coding sequence; and 2) at least one second protein(s) of interest, different from the first protein(s), wherein the second protein(s) is expressed from a plasmid that is delivered to and/or taken up by the spore or delivered to or included in a composition comprising the recombinant expressing the protein(s) of interest. The second protein(s) (or polypeptide or peptide) is not expressed as a fusion protein and is not typically bound to the spore surface. Rather, the protein is expressed freely from the plasmid in, e.g., soluble form. The second protein(s) assembles onto or becomes associated with the first protein (e.g., first subunit) that is displayed on the spore surface, thereby producing complex multimeric protein structures (e.g., multiple different subunits) on the surface of spores.

These spores are useful for the delivery of all types of homomeric multimeric proteins, polypeptides, and peptides, and heteromeric multimeric proteins, polypeptides, and peptides, and combinations of such multimeric proteins, including, but not limited to, enzymes (e.g., dioxygenases, monooxygenases), co-stimulatory molecules, antigens, antigenic determinants, adjuvants, carbohydrates, etc. Spores displaying such multimer proteins, polypeptides, and peptides are useful in the variety of industrial and medicinal applications described herein for recombinant spores, including industrial applications involving multimeric enzymes comprising multiple subunits, and therapeutic and prophylactic treatment applications involving administration of recombinant spores displaying co-stimulatory molecules, adjuvants, antigens, immunomodulators, etc. to a subject to treat diseases and/or disorders or protect against diseases and/or disorders (e.g., spore vaccines comprising homo- or hetero-multimeric antigen subunits, also including, but not limited to, co-stimulatory molecules, adjuvants, carbohydrates, etc.), enhance or inhibit an immune response (e.g., spore displaying homo- or hetero-multimeric co-stimulatory molecules, adjuvants (e.g., CT, HLT, etc.), carbohydrates, etc.

### Example 19

### Comparison of Respective Responses of Mouse Strains to Recombinant Spores of the Invention

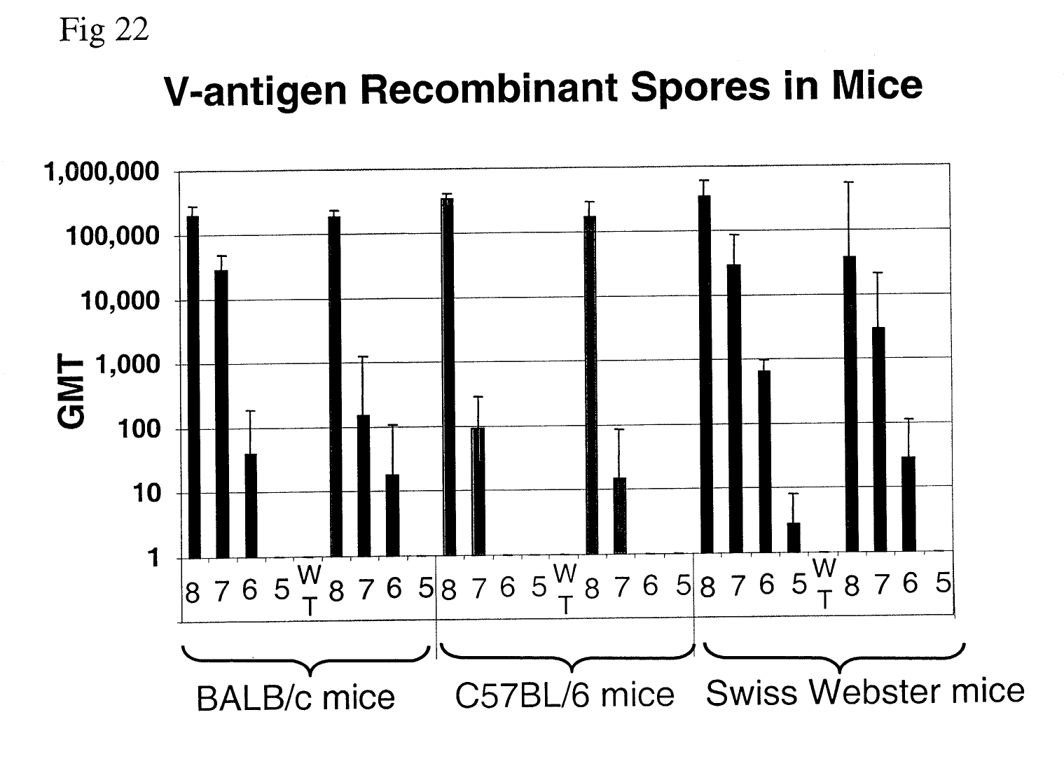

Two different inbred strains of mice (BALB/c and C57BL/6) and one strain of outbred mice (Swiss Webster) were compared for their respective responses to recombinant V antigen from Yersinia pestis fused to cotC coat protein and displayed on B. subtilis spores (FIG. 22). Mice (n=5) were inoculated once subcutaneously at day 1, followed by intraperitoneal boosts at days 21 and 35. Serum from each mouse was collected at day 45 and assayed for anti-Vantigen antibodies by a standard ELISA. Titers were expressed as geometric mean titers. Dose response was determined by inoculating each strain of mice with 1×10⁸, 1×10⁷, 1×10⁶, or 1×10⁵ spores at each injection timepoint. For each strain of mice, the experiment was done twice, with spores prepared at different times, to test for reproducibility. Mice were also injected with 1×10⁸ wildtype (WT) spores to test for background reactivity. The numbers on the X-axis in FIG. 22 are the exponent numbers of spores used per injection. Each bar in the graph represents 5 mice. All strains of mice responded similarly at the high dose to V antigen displayed on spores. At lower doses, the outbred mice responded somewhat better than did the inbred mice, indicating that the response to V antigen displayed on spores is not isolated to a single inbred mouse strain. Based on these data, we would also expect a robust response in other outbred mammals, such as humans.

### Example 20

### Methods for Generating Non-Viable Spores

Mutations that affect spore germination and spore viability are introduced into the genome of a Bacillus subtilis strain MW10 to generate a non-viable (non-germinating) spore. The resulting mutant strain is transformed with an expression plasmid encoding a molecule of interest. In this example, the mutant strain is transformed with an expression plasmid encoding an antigen of interest and then sporulated to produce antigen-coated mutant spores.

Three genes (e.g., sleB, cwlJ, cwlD) whose products are necessary for cortex hydrolysis during germination are knocked out using the method described in Paidhungat et al., J. Bacteriol. 183:4886-4893 (2001))was used in strain MW10. The coding region for each gene was replaced by a different antibiotic marker (e.g., tetracycline (tet), spectinomycin (spe), and erythromycin (erm) to show the gene is removed as described in Paidhungat et al. The resultant triple mutant (sleBA::tet cwlJA::spc cwlDA::erm) spores show a 10⁵-10⁶ fold lower germination than wild-type viable spores. As the typical vaccine dosage is about 10⁸ spores, the germination defect does not sufficiently reduce the number of viable spores per vaccine dose.

All publications, patents, patent applications, and other documents mentioned in the specification are indicative of the level of those skilled in the art to which this invention pertains. All publications, patents, patent applications, and other documents are herein incorporated by reference in their entirety for all purposes to the same extent as if each individual publication, patent, patent application, or other document was specifically and individually indicated to be incorporated herein by reference in its entirety for all purposes. Subheadings in the specification document are included solely for ease of review of the document and are not intended to be a limitation on the contents of the document in any way.

Although the foregoing invention has been described in some detail by way of illustration and example for purposes of understanding, it will be obvious that certain changes and modifications may be practiced within the scope of the appended claims.

That which is claimed:

1. A method for modulation of an immune response of an organism, said method comprising contacting said organism with a spore system comprising a recombinant spore having at least one exogenous nucleic acid, peptide, or polypeptide which modulates an immune response in the organism, wherein said spore is administered via a delivery system selected from the group consisting of respiratory delivery system, nasal delivery system, parenteral delivery system, and mucosal delivery system.

2. The method of claim 1, wherein said modulation is the production of an immune response.

3. The method of claim 1, wherein said modulation is the enhancement of an immune response.

4. The method of claim 1, wherein the nucleic acid, peptide, or polypeptide is displayed on or bound to a surface of the spore.

5. The method of claim 1, wherein the nucleic acid, peptide, or polypeptide is contained within the spore.

6. The method of claim 1, wherein said modulation results from the release of the nucleic acid, peptide, or polypeptide from the spore system.

7. The method of claim 1, wherein the spore of said sp ore system is a non-viable spore.

8. The method of claim 1, wherein the spore of said spore system is a bacterial spore.

9. The method of claim 1, wherein the at least one exogenous nucleic acid, peptide, or polypeptide comprises at least one immunomodulatory agent.

10. The method of claim 9, wherein said at least one immunomodulatory agent is selected from the group consisting of: cytokines, co-stimulatory agents, antigens, antibodies, adjuvants, and binding receptors.

11. The method of claim 10, wherein the at least one immunomodulatory agent comprises an antigen.

12. The method of claim 1, wherein the polypeptide or peptide is produced as a fusion protein with a spore coat protein.

13. The method of claim 12, wherein the spore coat protein is at least one protein selected from the group consisting of: CotA, CotB, CotC, CotD, CotE, CotF, CotG, CotN, CotS, CotT, CotV, CotW, CotX, CotY, and CotZ.

14. The method of claim 13, wherein the spore coat protein is CotC protein.

15. The method of claim 13, wherein the spore coat protein is CotD protein.

16. The method of claim 1, wherein the nucleic acid, peptide, or polypeptide is produced by vegetative cells produced by said spore following germination of said spore.

17. The method of claim 1, wherein said spore is delivered via the respiratory delivery system.

18. The method of claim 1, wherein said spore is delivered via the nasal delivery system.

19. The method of claim 1, where said spore is delivered via the parenteral delivery system.

20. The method of claim 1, wherein said polypeptide is displayed on the surface of the spore after lysis of the mother cell of said spore.

21. A method for modulation of an immune response of an organism, said method comprising contacting said organism with a spore system comprising a non-viable recombinant spore having at least one exogenous nucleic acid, peptide, or polypeptide which modulates an immune response in the organism.

22. The method of claim 21, wherein said nucleic acid, peptide, or polypeptide is displayed on or bound to a surface of the spore.

23. The method of claim 21, wherein said polypeptide is an antigen and said peptide is an antigenic peptide.

24. The method of claim 21, wherein said polypeptide, or peptide is produced as a fusion protein with a spore coat protein.

25. The method of claim 24, wherein said spore coat protein is at least one protein selected from the group consisting of: CotA, CotB, CotC, CotD, CotE, CotF, CotG, CotN, CotS, CotT, CotV, CotW, CotX, CotY, CotZ.

26. A composition comprising a spore system, said spore system comprising a spore and at least one exogenous nucleic acid molecule, peptide, or polypeptide displayed on, bound to, or contained within said spore wherein said nucleic acid, peptide, or polypeptide modulates an immune response when administered to an organism via the respiratory delivery system, nasal delivery system, or the parenteral delivery system.

27. The composition of 26, wherein the nucleic acid, peptide, or polypeptide is displayed on or bound to the surface of the spore.

28. The composition of 26, wherein the peptide or polypeptide is produced as a fusion protein with a spore coat protein.

29. The composition of 28, wherein the spore coat protein is at least one protein selected from the group consisting of: CotA, CotB, CotC, CotD, CotE, CotF, CotG, CotN, CotS, CotT, CotV, CotW, CotX, CotY, and CotZ.

30. A composition comprising a spore system, said spore system comprising a spore, at least one antigen, and at least one adjuvant and/or co-stimulatory polypeptide.

31. The composition of claim 30, wherein said at least one antigen is displayed on or bound to the surface of the spore.

32. The composition of claim 30, wherein said at least one antigen is contained within the spore.

33. The composition of claim 30, wherein said spore of said spore system is a non-viable spore.

34. The composition of claim 30, wherein said at least one adjuvant or co-stimulatory polypeptide is displayed on, bound to, or contained within said spore.

35. The composition of claim 30, wherein said at least one antigen is selected from the group comprising peptides, polypeptides, proteins, carbohydrates, or nucleotide sequences of interest.

36. The composition of claim 30, wherein said at least one antigen is a fusion protein comprising a spore coat polypeptide.

37. The composition of claim 30, wherein said at least one adjuvant is a fusion protein comprising a spore coat polypeptide.

38. The composition of claim 30, wherein said at least one co-stimulatory polypeptide is a fusion protein comprising a spore coat polypeptide.

39. The composition of claim 30, wherein said composition further comprises a carrier.

40. The composition of claim 39, wherein said carrier is a fluid.

41. The composition of claim 39, wherein said carrier is an excipient.

42. A method for releasing a spore system of interest, said method comprising:
a) transforming a cell that is capable of sporulation with an exogenous nucleic acid molecule;
b) inducing sporulation of the cell, whereby at least one spore system is produced, said spore system comprising said nucleic acid molecule and/or any polypeptide produced therefrom, and a spore; and
c) lysing the cell to release said spore system.

43. The method of claim 42, wherein said exogenous nucleic acid molecule encodes a polypeptide.

44. The method of claim 43, wherein said polypeptide is displayed on or bound to a surface of the spore.

45. The method of claim 43, wherein said polypeptide is contained within said spore.

46. The method of claim 43, wherein the polypeptide is a fusion protein comprising a spore coat protein of the spore.

47. The method of claim 42, wherein said cell that is capable of sporulation is a bacterial cell.

48. The method of claim 42, wherein said cell that is capable of sporulation is a fungal cell.

49. The method of claim 48, wherein said fungal cell is a yeast cell.

50. A method for displaying a polypeptide at one or more sites of interest on a surface of a spore, said method comprising:
a) transforming a cell that is capable of sporulation with a recombinant nucleic acid vector comprising a nucleic acid molecule encoding a polypeptide fused in frame to a nucleic acid molecule encoding a spore coat protein; and
b) expressing a fusion protein comprising said polypeptide and said spore coat protein such that said fusion protein is attached to the spore coat of the spore at one or more sites of interest on the surface of the spore.

51. The method of claim 50, wherein said spore coat protein is selected from the group consisting of: CotA, CotB, CotC, CotD, CotE, CotF, CotG, CotN, CotS, CotT, CotV, CotW, CotX, CotY, and CotZ.

52. A detection system comprising a spore system wherein said spore system comprises a moiety that provides a detectable signal and a polypeptide displayed on, bound to, or contained within the spore system, wherein said polypeptide is capable of capturing a detectable compound.

53. The detection system of claim 52, wherein said polypeptide is selected from the group consisting of: an antibody, a ligand, an antigen, a receptor, an epitope, and an enzyme.

54. The detection system of claim 52, wherein said moiety is comprised of a chromophore or fluorophore.

55. A method for detecting a compound, said method comprising contacting the detection system of claim 52 with the compound of interest.

56. The method of claim 55, wherein said compound is selected from the group comprising: an enzymatic substrate, an antibody, an antigenic agent, a ligand, and an antagonist.

57. A method for delivery of a polypeptide of interest, said method comprising:
a) transforming a cell that is capable of sporulating with a nucleic acid sequence encoding said polypeptide;
b) inducing sporulation of said cell to form a spore; and
c) delivering said spore to a site of interest.

58. The method of claim 57, wherein said polypeptide is displayed on or bound to the surface of said spore.

59. The method of claim 57, wherein said polypeptide is contained within said spore.

60. The method of claim 57, wherein said spore is delivered as an intact spore.

61. The method of claim 57, wherein said spore is delivered as a germinated spore.

62. The method of claim 57, wherein said spore is delivered as a replicating vegetative cell arising from a spore.

63. The method of claim 57, wherein said polypeptide is expressed as a fusion protein with a spore coat protein.

64. The method of claim 63, wherein said spore coat protein is selected from the group consisting of: CotA, CotB, CotC, CotD, CotE, CotF, CotG, CotN, CotS, CotT, CotV, CotW, CotX, CotY, and CotZ.

65. A method for generating a desired product comprising reacting a substrate with a spore system, said spore system comprising a recombinant spore having at least one polypeptide wherein said polypeptide has enzymatic activity.

66. The method of claim 65, wherein said spore comprises the enzymes needed to produce said desired product.

67. A composition comprising a spore system said spore system comprising a recombinant spore having at least one exogenous polypeptide wherein said polypeptide has enzymatic activity and a substrate wherein said enzyme alters said substrate.

68. A composition comprising a spore system, said spore system comprising a non-viable spore and at least one exogenous nucleic acid, peptide, or polypeptide displayed on, bound to, or contained within said spore.

69. The composition of claim 68, wherein the nucleic acid, peptide, or polypeptide is displayed on or bound to the surface of the spore.

70. The composition of claim 68, wherein the nucleic acid, peptide, or polypeptide is contained within the spore.

71. The composition of claim 68, wherein said spore system comprises more than one exogenous nucleic acid, peptide, or polypeptide associated with said spore.

72. The composition of claim 68, wherein at least one of said polypeptide has an enzymatic activity.

73. The composition of claim 68, wherein said composition further comprises a substrate which is capable of being altered by said enzymatic activity.

74. The composition of claim 68, wherein said spore system is immobilized by attachment to a solid support.

75. The composition of claim 74, wherein said solid support is selected from the group consisting of: beads, membranes, gels, microtiter plates, or vessels.

76. The composition of claim 68, wherein said composition further comprises a carrier.

77. The composition of claim 76, wherein said carrier is a fluid.

78. The composition of claim 76, wherein said carrier is an excipient.

79. A composition comprising a spore system, said spore system comprising a non-viable spore and one or more expression cassettes, wherein said one or more expression cassettes comprise a promoter operably linked to a nucleotide sequence of interest.

80. A composition of claim 79, wherein said nucleotide sequence of interest is operably linked to a nucleotide sequence encoding a spore coat protein.

81. A composition of claim 80, wherein said spore coat protein is selected from the group consisting of CotA, CotB, CotC, CotD, CotE, CotF, CotG, CotN, CotS, CotT, CotV, CotW, CotX, CotY, and CotZ.

82. A composition of claim 79, wherein the polypeptide encoded by the nucleotide sequence of interest is targeted to the spore coat.

83. A method for modulation of an adjuvant effect in an organism, said method comprising:
a) generating anon-viable spore, wherein said spore has an adjuvant effect;
b) isolating said spore; and
c) contacting said organism with said spore and a nucleic acid, peptide, or polypeptide.

84. The method of claim 83, wherein said spore is a recombinant spore.

85. The method of claim 83, wherein said spore is a non-recombinant spore.

86. A composition comprising a spore system, said spore system comprising a non-viable spore and one or more expression cassettes, wherein said expression cassettes are comprised of a promoter operably linked to a multiple cloning site.

87. A composition of claim 86, wherein said multiple cloning site is operably linked to a nucleotide sequence encoding a spore coat protein.

88. A composition comprising a spore system, said spore system comprising a spore and at least one streptavidin or avidin molecule displayed on or bound to said spore.

89. The composition of claim 88, wherein said spore of said spore system is a non-viable spore.

90. The composition of claim 88, wherein said composition is immobilized by attachment to a solid support.

91. The composition of claim 90, wherein said solid support is selected from the group consisting of: beads, membranes, gels, microtiter plates, or vessels.

92. The composition of claim 88, wherein said streptavidin or avidin molecule is a fusion protein with a spore coat protein.

93. A composition comprising a spore system, said spore system comprising a spore and at least one exogenous nucleic acid binding particle displayed on or bound to said spore.

94. The composition of claim 93, wherein said spore of said spore system is non-viable.

95. The composition of claim 93, wherein said nucleic acid binding particle is selected from the group consisting of peptides, polypeptides, proteins, or nucleic acid molecules.

96. The composition of claim 95, wherein said polypeptide is selected from the group consisting of HU and polylysine.

97. The composition of claim 93, wherein said nucleic acid binding particle is a fusion protein comprising a spore coat protein.

98. A composition comprising a spore system, said spore system comprising a spore and at least one peptide, polypeptide, protein, carbohydrate, or nucleotide sequence having anti-pathogenic activity displayed on, bound to, or contained within said spore.

99. The composition of claim 98, wherein said spore of said spore system is non-viable.

100. The composition of claim 98, wherein said peptide, polypeptide, or protein having anti-pathogenic activity is a fusion protein comprising a spore coat protein.

101. A method of enhancing an immune response to an immunogenic polypeptide or peptide in a subject, said method comprising administering to the subject a population of spores and an expression vector comprising a nucleotide sequence encoding the immunogenic polypeptide or peptide, wherein the immune response is enhanced compared to the immune response generated by administration of the expression vector or encoded immunogenic polypeptide or peptide alone to the subject.

102. The methods of claim 101, wherein the immunogenic polypeptide or peptide comprises an antigen.

103. The method of claim 101, wherein the enhanced immune response comprises increased antibody production.

104. The method of claim 101, wherein the population of spores comprises non-viable or non-germinating spores.

105. The method of claim 101, wherein the spores have an adjuvant effect.

106. A method of enhancing an immune response to an immunogenic polypeptide or peptide in a subject, said method comprising administering to the subject a population of spores and an immunogenic polypeptide or peptide, wherein the immune response to the immunogenic polypeptide or peptide is enhanced compared to the immune response generated by administration of the immunogenic polypeptide or peptide alone to the subject.

107. The method of claim 106, wherein the enhanced immune response comprises an increased production of antibodies specific to the immunogenic polypeptide or peptide.

108. The method of claim 107, wherein the immunogenic polypeptide or peptide is an antigen.

109. The method of claim 106, wherein the population of spores comprises non-viable or non-germinating spores.

110. The method of claim 106, wherein the spores act as adjuvants to enhance the immune response.

111. A composition comprising a spore system, said spore system comprising at least two spores wherein each spore displays a different peptide, polypeptide, or protein.

112. The composition of claim 111, wherein said spore is non-viable.

113. The composition of claim 111, wherein said polypeptide is a fusion protein comprising a spore coat protein.

114. The composition of claim 111, wherein said polypeptide is selected from the group consisting of: antigens, adjuvants, co-stimulatory agents, and immunomodulatory agents.

115. The composition of claim 111, wherein said spore system modulates more than one immune response in an organism.

116. The composition of claim 115, wherein said immune responses are to different antigens.

117. A composition comprising a spore system, said spore system comprising a spore and at least one rotavirus capsid protein displayed on, bound to, or contained within said spore.

118. The composition of claim 117, wherein said rotavirus capsid protein is selected from the group consisting of: VP4, VP6, and VP7.

119. The composition of claim 118, wherein said rotavirus capsid protein is VP6.

120. The composition of claim 117, wherein said spore is non-viable.

121. The composition of claim 117, wherein said spore system modulates an immune response when administered to an organism.


SEQUENCE LISTING (6 sequences)

SEQ ID NO: 1 - DNA, 258 bp, Bacillus subtilis
  CDS (1)..(258): CotC27 including HA11 epitope region
atgggttattacaaaaaatacaaagaagagtattatacggtcaaaaaaacgtattataagaagtattacgaatatgataaatctagaggtacctgctatccttatgatgttcctgattatgcttctttaggatccctgcagaaagattatgactgtgattacgacaaaaaatatgatgactatgataaaaaatattatgatcacgataaaaaagactatgattatgttgtagagtataaaaagcataaaaaacactac

SEQ ID NO: 2 - protein, 86 aa, Bacillus subtilis
  CotC27 including HA11 epitope region
MGYYKKYKEEYYTVKKTYYKKYYEYDKSRGTCYPYDVPDYASLGSLQKDYDCDYDKKYDDYDKKYYDHDKKDYDYVVEYKKHKKHY

SEQ ID NO: 3 - DNA, 639 bp, Bacillus circulans
  CDS (1)..(639): lipase 396
atgaaatttataaaaagaaggatcattgcacttgtaacaattttggtgctgtcagtcacatcgctgtttgcgatgcagccgtcagcaaaagccgctgaacacaatccagttgttatggttcacggtatcggaggagcttcatacaattttgcgggaattaagagctatctcgtatctcagggctggtcacggggcaagctgtatgcggttgatttttgggacaagacagggacgaattataacaatggcccggtattatcacgatttgtgcaaaaggttttagacgaaacgggtgcgaaaaaagtggatattgtcgctcacagcatgggtggcgcgaacacactttactacataaaaaatctggacggcggaaataaaattgaaaacgtcgtaacgcttggcggcgcgaaccgtttgacgacaagcaaggcgcttccgggaacagatccaaatcaaaagattttatacacatccatttacagcagtgccgatatgattgtcatgaattacttatcaaaattagacggtgctaaaaacgttcaaattcatggcgttgggcacattggtttattgatgaacagccaagtcaacagcctgattaaagaaggactgaacggcgggggcctcaatacaaattaa

SEQ ID NO: 4 - protein, 212 aa, Bacillus circulans
  lipase 396
MKFIKRRIIALVTILVLSVTSLFAMQPSAKAAEHNPVVMVHGIGGASYNFAGIKSYLVSQGWSRGKLYAVDFWDKTGTNYNNGPVLSRFVQKVLDETGAKKVDIVAHSMGGANTLYYIKNLDGGNKIENVVTLGGANRLTTSKALPGTDPNQKILYTSIYSSADMIVMNYLSKLDGAKNVQIHGVGHIGLLMNSQVNSLIKEGLNGGGLNTN

SEQ ID NO: 5 - DNA, 29 bp, Artificial Sequence
  Description of Artificial Sequenceprimer for fusion protein
atatctgcagatttgtattgaggcccccg

SEQ ID NO: 6 - DNA, 32 bp, Artificial Sequence
  Description of Artificial Sequenceprimer for terminator
atatctgcagttaatttgtattgaggcccccg